(19) United States
(12) Patent Application Publication
(10) Pub. No.: US 2017/0348414 A1
(43) Pub. Date: Dec. 7, 2017

(54) NOVEL SYNTHETIC ANTICANCER, ANTIFUNGAL, AND ANTIBACTERIAL VACCINES

(71) Applicant: Wayne State University, Detroit, MI (US)

(72) Inventors: Zhongwu Guo, Northville, MI (US); Guochao Liao, Detroit, MI (US); Zhifang Zhou, Detroit, MI (US); Mohabul Mondal, Detroit, MI (US); Srinivas Burgula, Detroit, MI (US)

(73) Assignee: Wayne State University, Detroit, MI (US)

(21) Appl. No.: 15/510,932

(22) PCT Filed: Sep. 14, 2015
(86) PCT No.: PCT/US2015/049987

§ 371 Date: Mar. 13, 2017

Related U.S. Application Data
(60) Provisional application No. 62/050,522, filed on Sep. 15, 2014.

(51) Int. Cl. A61K 39/385; A61K 39/00; A61K 39/02; A61K 47/64; A61K 39/095; A61K 39/145; C12N 7/00

(52) U.S. Cl. CPC A61K 39/385; A61K 39/145; A61K 39/00; A61K 39/0011; C12N 7/00; A61K 47/646; A61K 39/095; A61K 39/02; A61K 2039/6018; C12N 2760/16222; C12N 2760/16234

(57) ABSTRACT

Described herein are compounds for use in vaccine compositions which contain natural or synthetic carbohydrate antigens. Such vaccines may be highly immunologically active due to the conjugation with an immune-stimulating protein or with a monophosphorylated lipid A derivative, and may be self-adjuvanting due to the presence of a monophosphorylated lipid A derivative. Treatments for cancer and fungal and bacterial infections are described herein.

### CROSS REFERENCE TO RELATED APPLICATIONS

This application is related to and claims the benefit of priority to U.S. Provisional Patent Application No. 62/050,522, filed Sep. 15, 2014, the content of which is hereby incorporated by reference in its entirety.

### FEDERALLY SPONSORED RESEARCH OR DEVELOPMENT

This invention was made with government support under grant number R01 CA095142 awarded by the National Institutes of Health (NIH). The U.S. government has certain rights to the invention.

### BACKGROUND

The abnormal glycans expressed by cancer cells, known as tumor-associated carbohydrate antigens (TACAs), are useful epitopes for the development of therapeutic cancer vaccines, as they are abundant and exposed on the cancer cell surface and thereby easy targets for the human immune system. Among many TACAs identified so far, the globo H antigen, which is a rather tumor-specific hexasaccharide antigen, is especially attractive. Globo H was first discovered in conjugation with lipids on human breast cancer cell MCF-7, and later on was also found on a variety of other epithelial tumors, such as lung, colon, ovarian, and prostate cancer. As a result, globo H-based anticancer vaccines can be broadly useful for treating different tumors.

However, similar to most carbohydrate antigens, globo H itself is poorly immunogenic and T cell-independent, while T cell-mediated immunity, which means antibody affinity maturation and improved immunological memorization and cytotoxicity to cancer cells compared to purely humoral or antibody-mediated immunity, is critical for cancer immunotherapy. The conventional method to deal with the issue is to couple carbohydrate antigens with an immunologically active carrier protein to form protein conjugate vaccines, a strategy that not only increases the immunogenicity of carbohydrates but also switches them from T cell-independent to T cell-dependent antigens. The most commonly used carrier protein in the development of anticancer vaccines is keyhole limpet hemocyanin (KLH). The KLH conjugates of globo H have made great progress as therapeutic cancer vaccines. For example, used with an external adjuvant such as QS-21, they have been shown to elicit strong immune responses and thus have been in phase III clinical trials for the treatment of breast and prostate cancer, demonstrating the great potential of globo H-based vaccines for cancer immunotherapy.

Despite that the KLH conjugates of globo H as anticancer vaccines have shown promising results, there are still issues in their clinical application. The KLH-globo H conjugates usually provoked high levels of antigen-specific IgM antibodies, but the levels of IgG antibodies, which indicate T cell-mediated immunity, were relatively low in patients. This was probably due to the fact that the carrier protein itself could elicit strong immunity and thereby suppress the immune response to the carbohydrate antigen. Furthermore, due to the multivalent property of carrier proteins and the unpredictability of the conjugation reaction, it is difficult to control the coupling sites and the loading levels of carbohydrates in TACA-protein conjugates, causing problems in their quality control. In addition, traditional vaccines have to be used with an external adjuvant to be effective, which can lead to serious side effects. For example, inflammatory responses at the injection site and systemic syndromes such as fever, arthralgias, and myalgias induced by QS21 have been reported during the clinical trials of KLH-globo H conjugates.

To overcome these problems associated with protein-TACA conjugates and develop effective cancer vaccine based on globo H, we have invented a new type of fully synthetic glycoconjugate vaccines by coupling globo H antigen with a glycolipid carrier, monophosphoryl lipid A. Theses vaccines possess homogeneous and defined chemical structures, which would not only streamline their characterization and quality control. Moreover, they also have self-adjuvanting properties. Thus, they are not only safe and effective but also can be used alone without an additional external adjuvant.

Fungal infection poses a great threat to the human health, and its cases grow rapidly year by year due to the limitations of current antifungal drugs and, especially, the emergence of drug-resistant strains. As a result, deep-seated infections in nosocomial settings have a high mortality even after treatment with antifungal drugs. Moreover, many commensal and opportunistic fungi, previously thought to be nonpathogenic, have emerged as pathogens in immunocompromised patients. To meet the urgent medical need for antifungal therapies, development of prophylactic and/or therapeutic antifungal vaccines is considered as one of the most attractive and appropriate strategies.

Beta-(1,3)-glucan (β-glucan) is an essential cell wall component of various fungi, and its structure has been established. Their main carbohydrate chain is composed of approximately 1500 β-1,3-linked glucose units, with ca. 40-50 additional short β-1,6- or β-1,3-glucans attached to the main chain glucose 6-O-positions as branches. This biopolymer is exposed on the surface of fungal cells and is functionally necessary, thus it is an excellent target antigen for the development of broadly useful antifungal vaccines. It has been demonstrated that conjugates of natural β-glucan could provoke immunogenic protection against Candida albicans in mice. Therefore, a series of experimental vaccines based on β-glucans, such as their conjugates with diphtheria toxin CMR197, have been explored and shown to elicit protections against Candida in a mouse model. Recent studies suggested that linear β-glucan and its short oligosaccharides could also elicit immune responses and protections against C. albicans. Thus, synthetic oligosaccharide derivatives of β-glucan can be used for the development of antifungal conjugate vaccines.

Developing conjugate vaccines using synthetic oligosaccharide antigens is a relatively new concept. This type of vaccines has some advantages. For example, their synthetic antigens have defined chemical structures, which would facilitate detailed immunological and structure-activity relationship studies to help gain more insights into the function of vaccines and optimize vaccine design. The reaction sites and/or linkage positions of the carbohydrate antigens are well defined and predictable, which would improve vaccine quality control. Oligomeric β-glucans, such as linear tetra, penta, hexa, dodeca and hexadeca, and a branched heptadeca oligosaccharides, as promising candidate antigens for vaccine design have recently synthesized by several groups. However, they are rarely conjugated with carrier molecules and investigated as vaccines, whereas most conjugate vaccines studied so far are made of heterogeneous natural 3-glucans or oligosaccharide mixtures derived from natural β-glucans through hydrolysis.

We have prepared a series of both linear and branched oligo-β-glucans and coupled them to carrier proteins or monophosoryl lipid A. The resultant conjugates were thoroughly studied and demonstrated to be a new type of potent antifungal vaccines.

With the rapid growth of drug-resistance, various bacterial infections, such as meningitis, have once again become a major threat to the human health. For infectious disease control, vaccination is considered an effective strategy, and for the development of antibacterial vaccines, the rich, exposed and conserved capsular polysaccharides (CPSs) on the bacterial cell surface are valuable antigens. However, typically, carbohydrates are weakly immunogenic and T cell-independent antigens, thus they need to be covalently coupled with immunologically active carriers to form conjugates that can elicit T cell-dependent immunity, long-term immunologic memory, and antibody maturation and isotype switch from IgM to IgG. In recent decades, antibacterial conjugate vaccines composed of polysaccharides and proteins have received great success, and their clinic use has kept many infectious diseases under control.

Despite the great success of polysaccharide-based glycoprotein vaccines, they have inherent problems. First, polysaccharides used to create vaccines are isolated from bacteria. Therefore, they are heterogeneous and easily contaminated. Moreover, they have to be activated before conjugation with carrier proteins, which can further diversify polysaccharide structures. Second, carbohydrate-protein conjugation is uncontrollable, affording complex mixtures, thus their composition and quality are difficult to duplicate. Third, the carrier proteins can induce strong B cell responses that may suppress the desired immune responses to carbohydrates.

To address this problem, fully synthetic vaccines made of structurally defined oligosaccharides and small molecule carriers, including peptides and lipids, have become an attractive strategy. Such vaccines not only possess homogeneous and defined structures and easy-to-control qualities but are also free of bacterial contamination.

To develop fully synthetic carbohydrate-based glycoconjugate antibacterial vaccines, we have synthesized a series of oligosaccharide analogs of several bacterial CPSs, coupled them to carrier proteins or a new carrier molecule, monophosphoryl lipid A (MPLA), and evaluated the resultantly conjugates immunologically. Based on these results, a number of new fully synthetic conjugate vaccines against Haemophilus influenza type b (Hib) and group C Neisseria meningitidis have been discovered.

It has been well known for many years that antibodies to the CPS PRP of Haemophilus influenza type b (Hib), a polymer of repeating ribosyl ribitol phosphate (RRP) units, are protective against meningitis and other invasive diseases caused by this bacterium. Four commercial Hib glycoprotein vaccines were developed using PRP conjugates with carrier proteins such as diphtheria toxoid (PRP-D), tetanus toxoid (PRP-T), HbOC, and PRP-OMP. However, these PRPs isolated from bacterial cell culture supernatants are heterogeneous or often contaminated with other antigenic components because of the difficulty of purifying by multi-stage process from natural source.

Several methods to successfully synthesize the fragment of the Hib capsular polysaccharide using solution or solid-phase techniques were reported. Verez et al have explored one-step polycondensation reaction with the use of H-phosphonate chemistry to afford synthetic RRP oligomers to form effective vaccines, but these oligomers were mixtures with an average of eight repeating units.

The conjugates containing synthetic oligomers of RRP as antigens have proven efficient in inducing immunogenic response in animals, and tetramer conjugates were more immunogenic than trimer conjugates. At the same time, natural pentamer of RRP also used in some of the licensed vaccines. However, Chong et al reported glycopeptides conjugates containing either the PRP pentamer or hexamer failed to elicit anti-PRP antibody response higher than those obtained with trimer.

We developed a new method to synthesize homogeneous and structurally well-defined PRP oligosaccharides, which were different from the mixtures reported in the literature, and couple them to carrier proteins to form conjugates. These conjugates were used to systematically investigate the structure-activity relationships between the length of the PRP oligomers and their immunogenicity. It was found that the protein conjugates of well-defined trimer, tetramer and pentamer fragment of PRP are potent vaccines to stimulate robust immune responses. Meanwhile, these oligomers were also coupled with monophosphoryl lipid A, a demonstrated strong immunostimulator, and the conjugates were found to elicit a promising immunogenic response as Hib vaccines.

Group C N. meningitidis is one of the bacterial strains mainly responsible for meningitis epidemics. The most characteristic CSP of group C N. meningitidis is α-2,9-ploysialic acid. Studies have shown that protein conjugates of natural α-2,9-ploysialic acid are could stimulate robust protective immune responses against group C N. meningitidis. Thus, current glycoconjugate vaccines used to fight group C N. meningitidis are consisting of carrier proteins and α-2,9-ploysialic acid.

Accordingly, we designed and synthesized a series of α-2,9-oligosialic acids and coupled them with a carrier molecules, including both proteins and monophosphoryl lipid A, to formulate glycoconjugate vaccines that were evaluated in mice. It was revealed that these oligosaccharide conjugates elicited strong immune responses that could target group C N. meningitidis cells, thus forming effective vaccines against group C meningitis.

### BRIEF SUMMARY

In one aspect, the present invention is a compound of compound of formula (I): (M-L-A) wherein M is selected from the group consisting of a protein and a lipid A derivative, L is a linker, and A is a carbohydrate antigen comprising fucose. Such a compound may be used for treating or preventing cancer in a patient.

In another aspect, the present invention is a compound of compound of formula (V): (M-L-E) wherein M is selected from the group consisting of a protein and a lipid A derivative, L is a linker, and A is a beta-glucan. Such a compound may be used for treating or preventing fungal infection in a patient.

In another aspect, the present invention is a compound of compound of formula (V): (M-L-E) wherein M is selected from the group consisting of a protein and a lipid A derivative, L is a linker, and A is an oligosialic acid. Such a compound may be used for treating or preventing a bacterial disease, particularly meningitis, in a patient.

In a further aspect, the present invention is a compound of compound of formula (V): (M-L-E) wherein M is selected from the group consisting of a protein and a lipid A derivative, L is a linker, and A is an oligoribosylribitol phosphate. Such a compound may be used for treating or preventing a bacterial disease, particularly influenza, in a patient.

### Definitions

The term “alkyl group” or “alkyl” includes straight and branched carbon chain radicals. For example, a “C1-6 alkyl” is an alkyl group having from 1 to 6 carbon atoms. Examples of C1-C6 straight-chain alkyl groups include, but are not limited to, methyl, ethyl, n-propyl, n-butyl, n-pentyl, and n-hexyl. Examples of branched-chain alkyl groups include, but are not limited to, isopropyl, tert-butyl, isobutyl, etc. Examples of alkylene groups include, but are not limited to, —CH₂—, —CH₂—CH₂—, —CH₂—CH(CH₃)—CH₂—, and —(CH₂)₁₋₃. Alkylene groups can be substituted with groups as set forth below for alkyl.

The term alkyl includes both “unsubstituted alkyls” and “substituted alkyls,” the latter of which refers to alkyl moieties having substituents replacing a hydrogen on one or more carbons of the hydrocarbon backbone (e.g., 1 to 5 substituents, 1 to 3 substituents, etc.). Such substituents are independently selected from the group consisting of: halo (I, Br, Cl, F), —OH, —COOH, trifluoromethyl, —NH₂, —OCF₃, and O—C₁-C₃ alkyl.

Typical substituted alkyl groups thus are 2,3-dichloropentyl, 3-hydroxy-5-carboxyhexyl, 2-aminopropyl, pentachlorobutyl, trifluoromethyl, methoxyethyl, 3-hydroxypentyl, 4-chlorobutyl, 1,2-dimethyl-propyl, and pentafluoroethyl.

“Halo” includes fluoro, chloro, bromo, and iodo.

Some of the compounds in the present invention may exist as stereoisomers, including enantiomers, diastereomers, and geometric isomers. Geometric isomers include compounds of the present invention that have alkenyl groups, which may exist as entgegen or zusammen conformations, in which case all geometric forms thereof, both entgegen and zusammen, cis and trans, and mixtures thereof, are within the scope of the present invention. Some compounds of the present invention have cycloalkyl groups, which may be substituted at more than one carbon atom, in which case all geometric forms thereof, both cis and trans, and mixtures thereof, are within the scope of the present invention. All of these forms, including (R), (S), epimers, diastereomers, cis, trans, syn, anti, (E), (Z), tautomers, and mixtures thereof, are contemplated in the compounds of the present invention.

The term “antibody” refers to a monomeric (e.g., single chain antibodies) or multimeric polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The term “antibody” also includes antigen-binding polypeptides such as Fab, Fab′, F(ab′)2, Fd, Fv, dAb, and complementarity determining region (CDR) fragments, single-chain antibodies (scFv), chimeric antibodies, and diabodies. The term antibody includes polyclonal antibodies and monoclonal antibodies unless otherwise indicated.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide antigen, refers to a binding reaction that is determinative of the presence of a specified protein. Typically, an antibody specifically binds an antigen when it has a Kd of at least about 1 μM or lower, more usually at least about 0.1 μM or lower, and preferably at least about 10 nM or lower for that antigen.

A variety of immunoassay formats (e.g., Western blots, ELISAs, etc.) may be used to select antibodies specifically immunoreactive with a particular protein. For example, solid-phase ELISA immunoassays are routinely used to select antibodies specifically immunoreactive with a protein (see, e.g., Harlow and Lane, Antibodies: A Laboratory Manual, New York: Cold Spring Harbor Press, (1990) for a description of immunoassay formats and conditions that can be used to determine specific immunoreactivity).

The term “patient” as used herein means a mammalian subject, preferably a human subject, that has, is suspected of having, or is or may be susceptible to a condition associated with cancer, fungal infection, or bacterial infection.

The term “treatment,” as used herein, covers any treatment of a disease in a mammal, such as a human, and includes: (a) preventing the disease from occurring in a subject which may be predisposed to the disease but has not yet been diagnosed as having it, i.e., causing the clinical symptoms of the disease not to develop in a subject that may be predisposed to the disease but does not yet experience or display symptoms of the disease; (b) inhibiting the disease, i.e., arresting or reducing the development of the disease or its clinical symptoms; and (c) relieving the disease, i.e., causing regression of the disease and/or its symptoms or conditions. Treating a patient's suffering from disease related to pathological inflammation is contemplated. Preventing, inhibiting, or relieving adverse effects attributed to pathological inflammation over long periods of time and/or are such caused by the physiological responses to inappropriate inflammation present in a biological system over long periods of time are also contemplated.

As used herein, a vaccine is “self-adjuvanting” if the molecule comprising the antigen provokes an immune response as measured by any immunological assay or in an animal or human being to which it has been administered without requiring co-administration of an auxiliary adjuvant.

As used herein, a vaccine is “synthetic” if each of the following portions of the vaccine, if used, are created by either an organic synthesis scheme or recombinant DNA or cloning techniques, rather than being purified from an organism which has made these components naturally: a carbohydrate antigen, a linker, a monophosphorylated lipid A derivative, and a carrier protein. In one non-limiting example, harvesting lipid A from cultured bacteria does not constitute a synthetic lipid A derivative, whereas using the synthetic schemes disclosed in U.S. Pat. No. 8,809,285 to generate a monophosphorlyated lipid A derivative from monosaccharide blocks which have in turn been generated from commercially available glucosamine would be considered synthetic, even if the glucosamine precursor was purified or otherwise derived from an organism that created it naturally. In another non-limiting example, harvesting globo H carbohydrates from a population of MCF-7 cells would not be synthetic, but using the schemes of FIGS. 9-12 of the present disclosure would be considered synthetic, regardless of the provenance of the starting materials.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

FIG. 1 illustrates the structure of MPLA-, KLH-, and HSA-globo H conjugates 1, 2, and 3 in accordance with the anticancer vaccine of the present invention;

FIG. 2 is a scheme illustrating the synthesis of the MPLA-globo H conjugate 1;

FIG. 3 is a scheme illustrating the synthesis of conjugates 2 and 3;

FIGS. 4-6 are graphical representations of immunological studies of conjugates 1 and 2 in accordance with the anticancer vaccine of the present invention;

FIG. 7 is fluorescence-assisted cell sorting (FACS) data associated with an immunological study of globo H conjugates 1 and 2;

FIG. 8 is a graphical representation of tumor cytotoxicity study of the antisera induced by globo H conjugates 1 and 2;

FIGS. 9-12 are schemes illustrating the synthesis of a globo H derivative according to another aspect for the present disclosure;

FIGS. 13-15 are schemes illustrating the syntheses of oligo-β-glucans and their conjugates in accordance with antifungal vaccines of the present application;

FIGS. 16-17 are graphical representations of immunological studies of oligo-β-glucan conjugates in accordance with antifungal vaccines of the present invention;

FIG. 18 is a survival curve associated with a fungal exposure challenge;

FIGS. 19-22 are schemes illustrating the syntheses of branched oligo-β-glucans and their conjugates in accordance with antifungal vaccines of the present invention;

FIG. 23 is a graphical representation of an immunological study of branched oligo-β-glucan conjugates in accordance with antifungal vaccines of the present invention;

FIG. 24 is a survival curve associated with a fungal exposure challenge;

FIGS. 25-33 are schemes illustrating the syntheses of Hib CPS carbohydrates and their conjugates in accordance with anti-Hib vaccines of the present invention;

FIGS. 34-37 are schemes illustrating the syntheses of group C N. meningitidis carbohydrates and their protein conjugates in accordance with anti-meningitis vaccines of the present invention;

FIGS. 38-40 are graphical representations of immunological and cell bacterial cell binding studies of group C N. menigitidis carbohydrate-protein conjugates in accordance with anti-meningitis vaccines of the present invention;

FIGS. 41-42 are schemes illustrating the syntheses of group C N. menigitidis carbohydrate-MPLA conjugates in accordance with anti-meningitis vaccines of the present invention; and

FIGS. 43-48 are graphical representations of immunological bacterial cell binding studies of group C N. menigitidis carbohydrate-MPLA conjugates in accordance with anti-meningitis vaccines of the present invention.

These figures are explained in detail in the following section.

### DETAILED DESCRIPTION OF THE DRAWINGS AND THE PRESENTLY PREFERRED EMBODIMENTS

A new class of carrier molecules, namely, 1-O-dephosphorylated monophosphoryl derivatives of lipid A, can be used for the development of fully synthetic glycoconjugate vaccines. Lipid A is the core hydrophobic domain of bacterial lipopolysaccharides (LPSs) and mainly responsible for the immunostimulatory activity of LPSs. Its monophosphoryl derivative, known as monophosphoryl lipid A (MPLA), also has very strong immunostimulatory activity. They act through interaction with toll-like receptor 4 (TLR4) to stimulate a downstream signaling cascade and eventually the production of cytokines and chemokines, such as tumor necrosis factor-α (TNF-α), interleukin-1β (IL-1β), IL-6, interferon-β (IFN-β), etc. Different from lipid A, however, MPLA is essentially nontoxic, and therefore has been recently approved for clinical use as a human vaccine adjuvant. MPLA conjugates of artificial TACA analogs could elicit robust immune responses in the absence of an external adjuvant, suggesting the potential of creating fully synthetic, self-adjuvanting glycoconjugate vaccines with MPLA as a carrier molecule. Application of MPLA to the development of vaccines based on synthetic oligosaccharides in natural forms against cancer, fungus and bacterium have not been reported previously, which is one of the central inventions of this patent application.

MPLA derivatives which are contemplated as being useful for an invention of the present disclosure, and synthetic schemes for making them, have been described in U.S. Pat. No. 8,809,285, to Guo, which is incorporated herein by reference in its entirety.

Briefly, in one instance, monophosphorylated lipid A may be represented by the following formula:

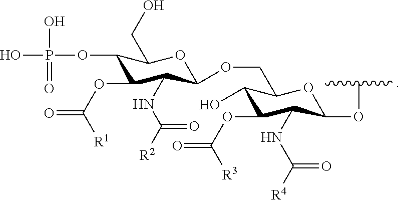

Wherein R¹ is —CH₂—CH(OR⁵)(CH₂)ₘCH₃, R⁵ is H or —C(O)—(CH₂)ᵣCH₃, m is an integer selected from 10 to 12, and n is 12; R² is —CH₂—CH(OR⁶)(CH₂)ₚCH₃, R⁶ is —C(O)—(CH₂)_{q}CH₃, wherein p is 10, and q is an integer selected from 10 to 12; R³ is —CH₂—CH(OR⁷)(CH₂)ᵣCH₃, R⁷ is H, and r is an integer selected from 8 to 10; R⁴ is —CH₂—CH(OR⁸)(CH₂)ₛCH₃, R⁸ is H or —C(O)—(CH₂)ₜCH₃, s is 10 or 11, and t is an integer selected from 11 to 13; or a pharmaceutically acceptable salt thereof.

In another embodiment, monophosphorylated lipid A is represented by the following formula:

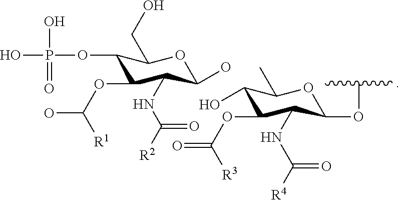

Wherein R¹ is —(CH₂)ₘCH₃, wherein m is an integer selected from 10 to 12; R² is —CH₂—CH(OR⁶)(CH₂)ₚCH₃, R⁶ is —C(O)—(CH₂)_{q}CH₃, wherein p is 10, and q is an integer selected from 10 to 12; R³ is —CH₂—CH(OR⁷)(CH₂)ᵣCH₃, R⁷ is H, and r is an integer selected from 8 to 10; R⁴ is —CH₂—CH(OR⁸)(CH₂)ₛCH₃, R⁸ is H or —C(O)—(CH₂)ₜCH₃, s is 10 or 11, and t is an integer selected from 11 to 13; or a pharmaceutically acceptable salt thereof.

Generally, the compound incorporating an MPLA derivative will be represented by the general Formula (I): M-L-X, wherein M represents the MPLA, L is a linker, and X is a carbohydrate antigen.

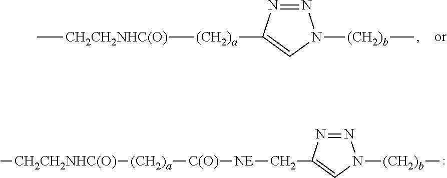

The linker may be any molecule which effectively joins the MLPA to the carbohydrate. In some specific instances, the linkers may be of the following constructions:

—(CH₂)₂—NHC(O)—(CH₂)ₐ—C(O)NH—(CH₂)_{b}—,

—(C₁-C₁₀ alkyl)-X—Y—(C₁-C₁₀ alkyl)-F-G-

In certain embodiments of Formula I, a and b are each 2. In other embodiments, F, G, X, and Y are each independently selected from the group consisting of C₁-C₁₀ alkyl, amide, carbonyl, alkene, cyano, phosphor, and thio.

Further examples of linkers include those represented by the following structures:

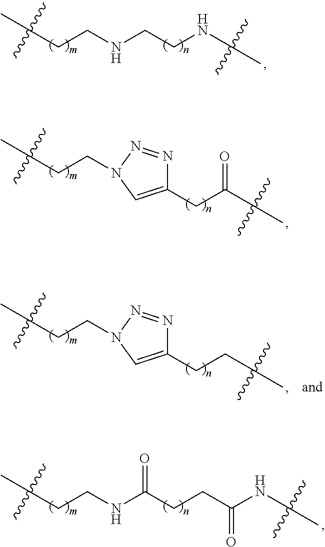

In such linkers, m and n can independently take on integer values from 1 to 30 inclusive. In some examples, m can equal 1, or 2, or 3, or 4, or 5, or 6, or 7, or 8, or 9, or 10. Similarly, n can equal 1, or 2, or 3, or 4, or 5, or 6, or 7, or 8, or 9, or 10.

In another embodiment, linkers can be created by joining several of the aforementioned linkers end-to-end. Two of the above linkers, with m and n for each linker selected independently, can be fused at their ends, or three linkers, or four or more linkers can also be fused. Any molecule which can effectly link the immunogenic molecule, such as lipid A derivatives or proteins, with the carbohydrate antigen, are viewed as acceptable for the present invention.

The carbohydrate antigens can be derived from a wide breadth of natural and synthetic molecules. These carbohydrates may be play roles in giving rise to immunity to cancer or form the basis of anticancer treatments; immunity to fungal infections or form the basis of antifungal treatments; or immunity to bacterial infections or form the basis of antibacterial infection treatments.

### Fucose-Containing Carbohydrates for Use in Cancer Vaccines and Therapies

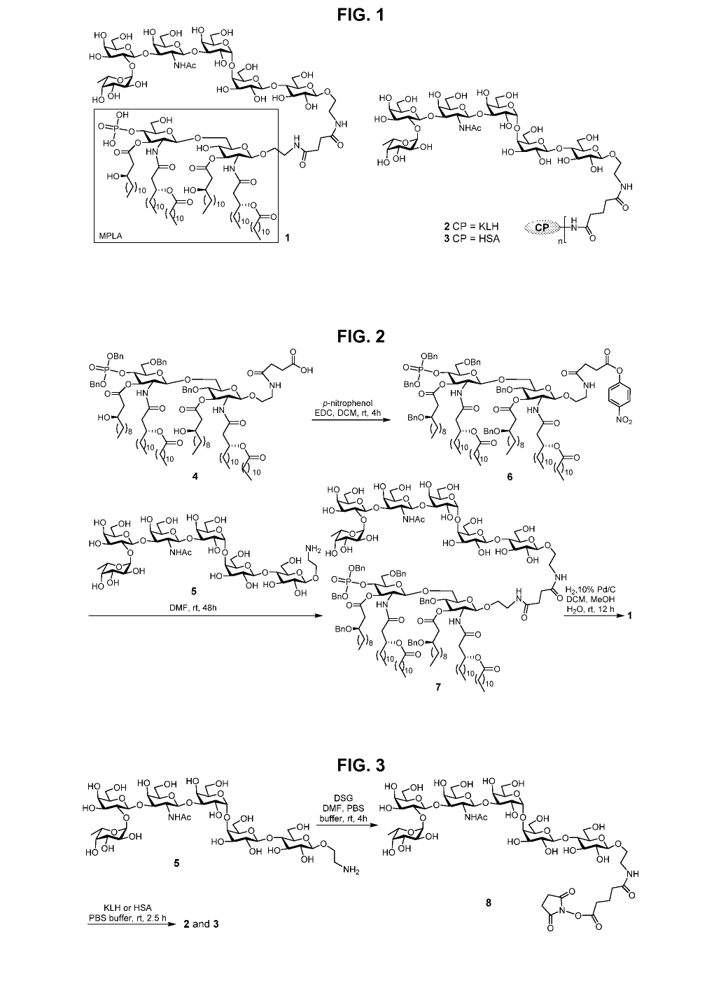

Overview: Developing fully synthetic anticancer vaccines based on globo H has been a challenge. For the purpose of this invention, globo H was synthesized and coupled with the synthetic monophosphoryl derivative of Neisseria meningitides lipid A—an optimized carrier molecule. The resultant glycoconjugate 1 (FIG. 1) was immunologically evaluated in mice. Its results were compared with that of the KLH-globo H conjugate 2 that was on clinical trial. In the meantime, the human serum albumin (HSA)-globo H conjugate 3 was also prepared and used as the coating antigen for enzyme-linked immunosorbent assays (ELISA) of globo H-specific antibodies.

The MPLA-globo H conjugate 1 was prepared by coupling a carboxylic acid derivative of N. meningitidis MPLA (4) with a derivative of globo H (5) that had a free amino group attached to its reducing end, according to the procedure outlined in FIG. 2. Compound 4 was converted into an activated ester 6 by reacting with p-nitrophenol and EDC hydrochloride. The activated ester 6 was then subjected to a regioselective reaction with 5 to afford the protected MPLA-globo H conjugate 7. Finally, all of the benzyl (Bn) groups in 7 were removed through hydrogenolysis to produce the desired MPLA-globo H conjugate 1 in a good overall yield (34%).

The KLH and HSA conjugates of globo H were readily prepared by coupling 5 with KLH and HSA through a bifunctional glutaryl linker (FIG. 3). Here, the glutaryl linker was selected because it provided reliable conjugation reactions. However, other linkers (see the claims) can also be used for this purpose. Treatment of 5 with 15 eq. of disuccinimidal glutarate (DSG) in DMF produced activated ester 8, which reacted with KLH or HSA in 0.1 M PBS buffer to afford glycoconjugates 2 and 3. After purification with a Biogel A 0.5 column, 2 and 3 were analyzed by the phenol-sulfuric acid method, with corresponding protein as control, to assess their carbohydrate loadings, which were 8% and 14%, respectively. The results showed that the coupling reactions were effective and the antigen loading levels were in the desired range (5-20%) for glycoconjugate vaccines or capture reagents used in ELISA. In addition, the HSA conjugate 3 was also analyzed with MALDI-TOF MS to obtain similar result (12%). On the other hand, the KLH conjugate 2, of which the molecular weight was too big for MS analysis, was studied with SDS-PAGE, and an increase in molecular weight of the glycoconjugate as compared to that of the protein itself proved the successful conjugation between KLH and globo H as well.

Immunological evaluation of the MPLA- and KLH-globo H conjugates 1 and 2 were carried out with female C57BL/6J mice. The MPLA conjugate 1 was administered alone without an external adjuvant in a liposomal formulation prepared with 1,2-distearoyl-sn-glycero-3-phosphocholine (DSPC) and cholesterol in a molar ratio of 10:65:50 by a reported method. By incorporating 1 in liposomes, we anticipated to improve not only its solubility to get a homogeneous formulation of 1 but also its immunogenicity. On the other hand, the KLH conjugate 2 was used as an emulsion with Freund's complete adjuvant (CFA) that is commonly used in animal study. In this case, 2 was first dissolved in PBS buffer and then thoroughly mixed with CFA before use.

For mouse immunization, 1 (16 μg of liposomes containing 5.4 μg of Globo H) and 2 (38 μg of adjuvant emulsion containing 3.1 μg of Globo H) were individually administered to each group of six mice through subcutaneous (s.c.) injection. Our studies have showed that the dosage of glycoconjugate vaccines within the range of 1-9 μg of carbohydrates had little impact on the induced antibody titers. The immunization schedule included boosting each mouse three times on days 14, 21, and 28, respectively, by injection of the same vaccine preparations after the initial immunization on day 1. Blood samples were collected from each mouse on day 0 before the initial inoculation (blank controls) and on days 21, 27, and 38 after immunizations. The blood samples were used to prepare sera according to standard protocols. The sera were then analyzed by ELISA using HSA-globo H conjugate 3 as the capture reagent to coat plates. The titers of both total antibodies (anti-kappa) and various antibody isotypes, including IgG1, IgG2b, IgG2c, IgG3, and IgM antibodies, were assessed. Here, IgG2c antibody, instead of IgG2a, was analyzed since C57BL/6 mouse was found to express IgG2c antibody instead of the allelic IgG2a antibody. For the analysis of antibody titers, ELISA plates were coated first with conjugate 3 and then with a blocking buffer [1% bovine serum albumin (BSA) in PBS]. Thereafter, half-log serially diluted mouse sera from 1:300 to 1:656100 in PBS were added to the plates. After incubation, the plates were washed and then incubated with 1:1000 diluted solutions of alkaline phosphatase (AP)-linked goat anti-mouse kappa, IgG1, IgG2b, IgG2c, IgG3, and IgM antibodies, respectively. Finally, the plates were developed with a p-nitrophenylphosphate (PNPP) solution, which was followed by colorimetric readout at 405 nm wavelength. Antibody titers were calculated from the curves obtained by drawing the adjusted optical density (OD) values, that is, after subtraction of the OD values of the blanks, against the serum dilution numbers and were defined as the serum dilution numbers yielding an OD value of 0.1.

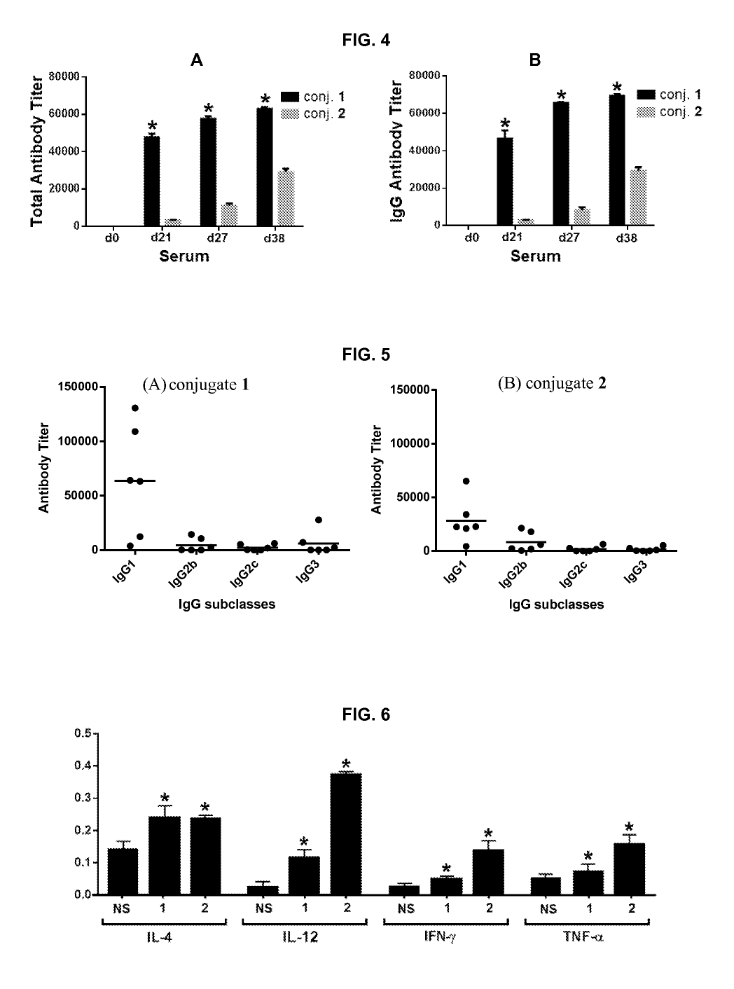

FIGS. 4A and 4B depict the overall total antibody titers and total IgG antibody titers of the pooled day 0, 21, 27, and 38 sera derived from each group of mice inoculated with conjugates 1 and 2, respectively. Clearly, the day 21 serum obtained from mice inoculated with the MPLA conjugate 1 twice on day 1 and day 14 already showed high globo H-specific total and IgG antibody titers (47,824 and 46,449, respectively), indicating that 1 could rapidly elicit robust immune responses. The anti-globo H antibody titers, especially the IgG antibody titers of the day 27 and 38 antisera (65,577 and 69,406, respectively), induced by 1 increased further after boost immunizations, suggesting the reinforcement of immune response against 1. The globo H-specific IgG antibody titers (2,783) of the day 21 antiserum of KLH conjugate 2 was about 17-fold lower than that of 1. After four immunizations, the IgG antibody titers induced by 2 was only 29,383, ca. 2.4-fold lower than that of 1. On the other hand, the titers of globo H-specific IgM antibodies induced by both conjugates were low.

FIGS. 4A and 4B show the overall total antibody and total IgG antibody titers, respectively, of pooled day 0, 21, 27, and 38 sera derived from mice immunized with conjugates 1 and 2. Antibody titers were defined as the serum dilution numbers yielding an OD value of 0.1, calculated from the curves obtained by drawing the OD values against the serum dilution numbers in the ELISA of mouse sera. The mean of antibody titers of three parallel experiments is shown for each sample, and the error bar shows the standard error of mean (SEM) of three replicate experiments. *Compared to the serum obtained on the same day after immunization with conjugate 2, the difference in antibody titers is statistically significant (student's t test, P<0.05).

FIGS. 5A and 5B depict the ELISA results about various subclasses of anti-globo H IgG antibodies in the day 38 antiserum of each individual mouse inoculated with glycoconjugate 1 or 2, as well as the group average. It was clear that conjugates 1 and 2 induced the similar patterns of immune responses, in both cases mainly IgG1 antibody (titers: 63,813 for 1 and 28,237 for 2), as well as a lower level of IgG2b antibody (titers: 4,578 for 1 and 8,294 for 2). Additionally, conjugate 1 also elicited some IgG3 antibody (titer: 6,159), which is typical with MPLA conjugates.

The IgG antibody titers of individual antiserum collected from mice immunized with conjugates 1 (FIG. 5A) and 2 (FIG. 5B). Each dot represents the result of one mouse and the horizontal bar represents the average antibody titer for each group of six mice.

The release of cytokines provoked by conjugates 1 and 2 was also analyzed. As depicted in FIG. 6, increased IL-4 expression indicates the activation of Th2 cell. This result is consistent with that of ELISA (FIGS. 5A and 5B). On the other hand, increased IL-12 expression suggests NK cell activation, and IFN-γ and TNF-α are produced by Th1 and/or CD8 cytotoxic T cells, which can activate macrophages and induce Ig antibody switch. Overall, the results of cytokine release analysis indicated that conjugates 1 and 2 induced T cell-mediated immunities.

FIG. 6 shows relative intensities of IL-4, IL-12, IFN-γ, and TNF-α in the pooled normal mouse sera (NS) and the pooled day 38 antisera from mice immunized with 1 and 2, respectively. The error bar represents the SD of two parallel experiments. A star indicates that compared to NS, the difference is statistically significant.

The above results proved that the MPLA-globo H conjugate 1 could effectively elicit robust immune responses against globo H in mice in the absence of an external adjuvant and that it could elicit significantly faster and stronger immune responses than the corresponding KLH conjugate currently in clinical trial for cancer immunotherapy. The patterns of immune responses elicited by conjugates 1 and 2 were similar, namely that both elicited mainly IgG1 antibodies and some IgG2b antibodies, which is a good indication of T cell-mediated immunities. This conclusion was also supported by the results of cytokine analysis of the pooled antisera derived from mice immunized with conjugates 1 and 2. Moreover, the IgG antibody titers increased with the number of boost immunizations for both glycoconjugates. The elicitation of strong IgG antibody responses and T cell-dependent immunities is critical for the therapeutic efficacy of cancer vaccines, since this is associated with antibody affinity maturation, improved antitumor activity, and long-term immunological memory. The significantly stronger T cell-dependent and IgG antibody immune responses induced by 1, as compared to the KLH conjugate 2, suggested the promise of 1 as a therapeutic cancer vaccine.

Antiserum binding to cancer cells. The capabilities of antisera obtained with conjugates 1 and 2 to recognize and bind to target cancer cells were investigated by the fluorescence-activated cell sorting (FACS) technology. Breast cancer cell MCF-7, which expresses globo H, was used in this study, with melanoma cell SKMEL-28 that does not express globo H as a negative control. These two cell lines were individually cultured with normal mouse serum (the negative control) or antisera derived from mice immunized with 1 and 2. Thereafter, cancer cells were incubated with fluorescein isothiocyanate (FITC)-labeled goat anti-mouse kappa antibody and were finally subjected to FACS analysis.

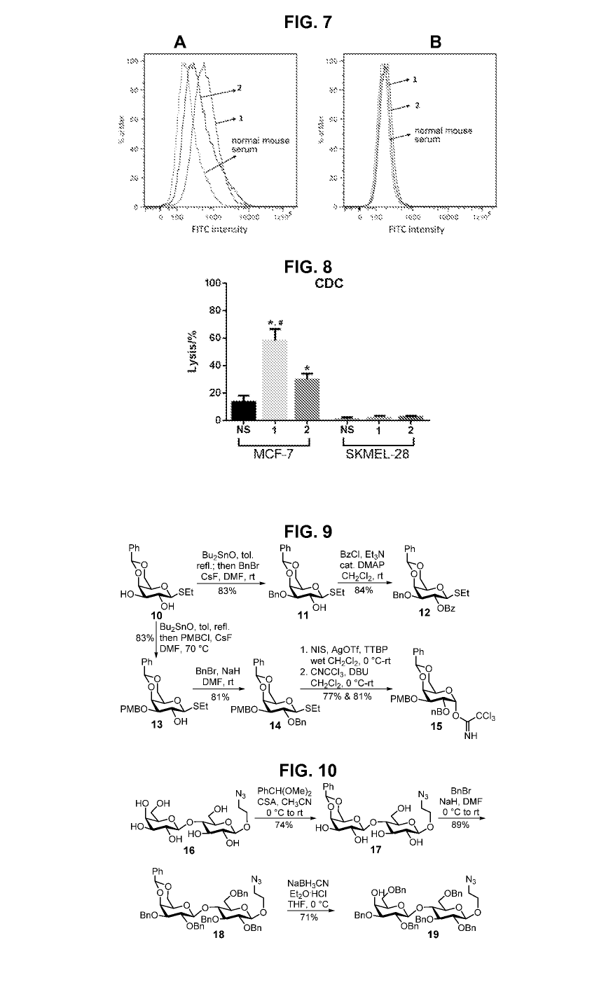

As depicted in FIG. 7A, significant fluorescent peak shifts to the right were observed with MCF-7 cell treated with anti-1 and anti-2 sera as compared to the cell treated with normal mouse serum. In contrast, the fluorescent profiles of SKMEL-28 cell treated with normal mouse serum and with anti-1 and anti-2 sera did not exhibit a significant difference (FIG. 7B).

FACS assay results of the binding between MCF-7 (FIG. 7A) or SKMEL-28 (FIG. 7B) cancer cell and normal mouse serum (labeled normal mouse serum), pooled antisera derived from mice immunized with conjugate 1 (curve labeled 1) or pooled antisera derived from mice immunized with conjugate 2 (curve labeled 2), respectively.

These results demonstrated that the antibodies elicited by conjugates 1 and 2 could specifically target and bind to globo H-expressing cancer cells but not cells that do not express globo H. Furthermore, the median fluorescence intensity (MFI) of MCF-7 cells treated with anti-conjugate 1 serum (MFI: 580) was significantly higher than that of MCF-7 cells treated with anti-conjugate 2 serum (MFI: 367) (FIG. 7A), indicating increased binding events and/or affinity of antibodies in anti-1 serum. This result was consistent with the ELISA results described above, namely that the antisera obtained with 1 had much higher antibody titers than the antisera obtained with 2, and thereby had provided another piece of evidence supporting the conclusion that 1 could induce significantly stronger immunological responses in mice than 2.

Antibody-mediated complement-dependent cytotoxicity (CDC) to cancer cells. The anticancer activities mediated by antisera derived from mice inoculated with conjugates 1 and 2 were also evaluated with cancer cells MCF-7 and SKMEL-28. In this study, cancer cells were cultured with normal mouse serum or with the above-mentioned antisera in the presence of rabbit complements, and the induced cell lysis was then analyzed by the lactate dehydrogenase (LDH) assay.

As depicted in FIG. 8, under the non-optimized condition, the lysis rates of MCF-7 cell mediated by anti-1 and anti-2 sera were about 60% and 30%, respectively. In contrast, under the same condition, no antibody-mediated cytotoxicity to SKMEL-28 cell was observed. The results confirmed that the antisera raised by conjugates 1 and 2 mediated effective and specific CDC to cancer cells which express the globo H antigen. The results in FIG. 8 further demonstrated that anti-1 sera mediated significantly stronger CDC to MCF-7 cell than anti-2 sera under the same condition, supporting that conjugate 1 may be a better vaccine than conjugate 2 for cancer immunotherapy.

The results of antibody-mediated complement-dependent cytotoxicity to MCF-7 and SKMEL-28 cells, shown as cell lyses caused by treatment with complements and normal mouse serum (NS), pooled antisera derived from mice immunized with 1 or pooled antisera derived from mice immunized with 2. The error bar shows the standard deviation of six parallel experiments. *Compared to the result of NS, the difference is statistically significant (P<<0.01); ^{#}compared to the result of anti-2 sera, the difference is statistically significant (P<<0.01).

All above studies have indicated that the globo H-MPLA conjugate 1, as well as its analogs, is a promising vaccine for cancer immunotherapy and it is worth further investigation and development for the treatment of breast, lung, colon, ovarian, and prostate cancer. Such analogs include fucose-containing carbohydrates including, but not limited to, globo series TACAs such as 43-9F antigen and lacto series TACAs including Le^{a}, Le^{b}, Le^{x}, Le^{y}, and Y2. Moreover, this new type of cancer has a number of advantages over traditional protein-TACA design. In addition to the conventional advantages of fully synthetic vaccines, such as well-defined structures, convenient characterization and easy quality control, the MPLA-globo H conjugate 1 had also exhibited some other useful properties as a therapeutic cancer vaccine. First, it elicited a faster and stronger immune response than the corresponding KLH conjugate 2. A robust immune response against globo H was established in mice after immunization with 1 twice, while it took four times of immunization with 2 to develop a solid immune response, and under such condition the titers of induced globo H-specific antibodies were still significantly lower than that induced by 1. A proposed explanation for this was that the strong immune response against KLH (the KLH-specific antibody titer was 293,919, ca. 12.7-fold higher than the globo H-specific antibody titer 23,177, Supporting Information) might have suppressed the immune response to the carbohydrate antigen. However, as a carrier molecule MPLA did not have this problem, since the MPLA-specific antibody titer (59,666) induced by the MPLA conjugate 1 was not significantly different from the globo H-specific antibody titer (63,038, Supporting Information). Second, the MPLA-globo H conjugate 1 was self-adjuvanting, thus it could be utilized alone without the use of an external adjuvant. This would not only simplify its clinical application but also help stabilize its property and function, and reduce side effects. Third, similar to the KLH conjugate 2, 1 also elicited T cell-dependent immunity, which is highly desirable for therapeutic cancer vaccines. Furthermore, the antisera induced by 1 had significantly stronger binding to and CDC against the globo H-expressing MCF-7 cancer cell than the antisera induced by the KLH conjugate 2 under the specific experimental conditions.

Beyond globo H, numerous other carbohydrate antigens containing fucose are contemplated as carbohydrates which can be attached to MLPA in accordance with the embodiments of the present invention. Our in-depth structure-activity relationship analysis of globo H-based glycoconjugate vaccines revealed that the L-fucose residue in the structure of globo H was critical to its immunogenicity and its strong binding to related antibodies. The result indicated that the presence of the fucose residue in globo H made its MPLA conjugate particularly immunogenic to form unexpectedly good vaccines, as compared to other MPLA conjugates (composed of sTn and GM3 antigens) as cancer vaccines previously prepared in our lab. The same strategy can be applied to developing cancer vaccines using other fucose-containing tumor-associated carbohydrate antigens (TACAs). These antigens include another globo series antigen 43-9F and lacto series antigens such as Le^{a}, Le^{b}, Le^{X}, Le^{y}, and Y2, as well as the hybrids.

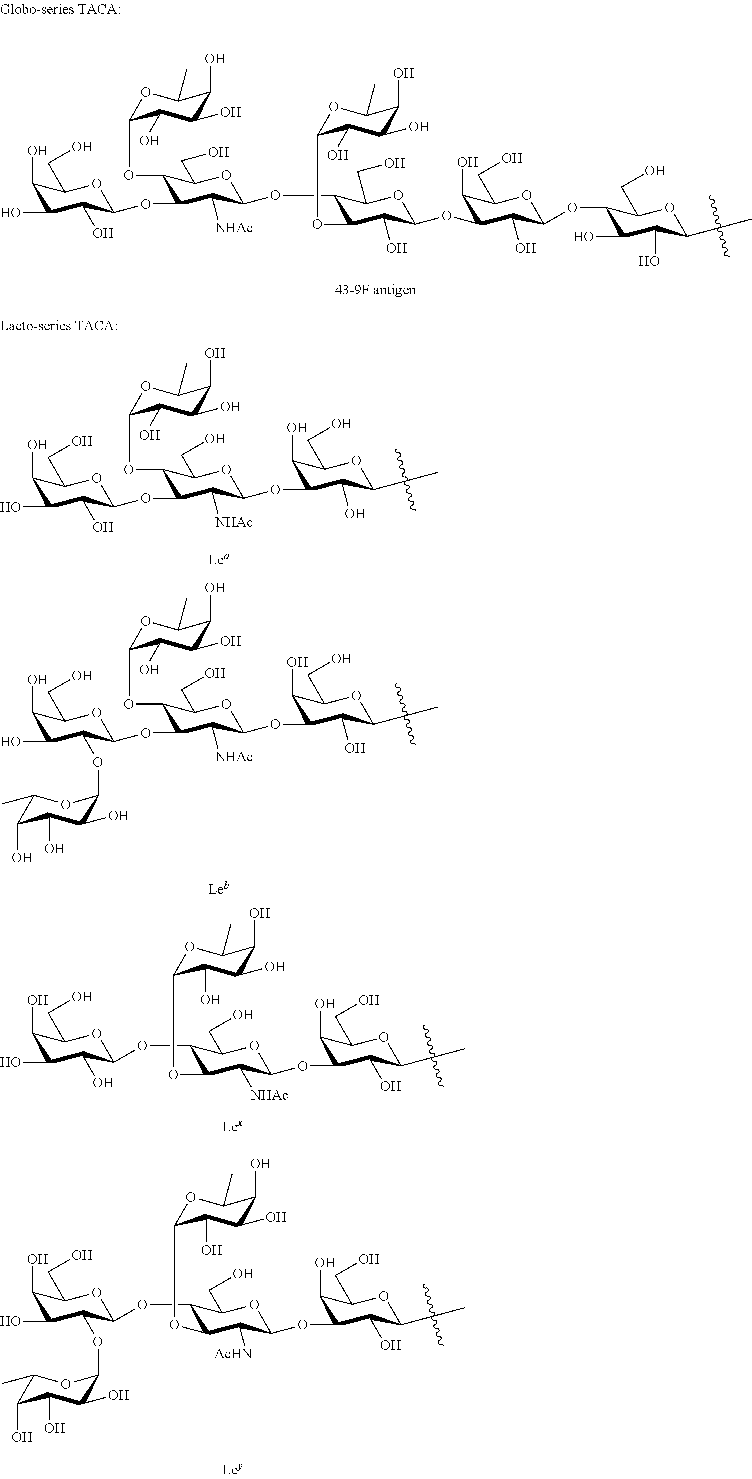

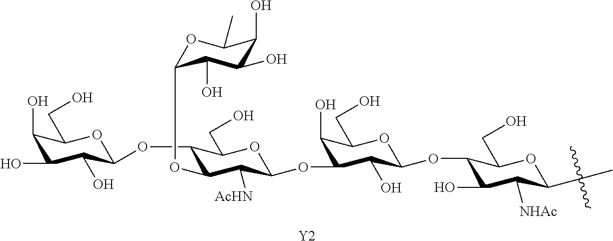

Any of globo-H, 43-9F antigen, Lea, Leb, Lex, Ley, and Y2, alone or in combination, can be conjugated to MPLA in accordance with the principles of this disclosure.

This invention also encompasses a method of synthesizing a synthetic globo H. In an effort to explore TACA-based anticancer vaccines, described herein an efficient synthesis for a globo H derivative 5 (FIG. 2), which carried a free amino group at the glycan reducing end. It would facilitate the conjugation of this carbohydrate antigen with other molecules, such as vaccine carriers like KLH or monophosphoryl lipid A derivatives—a new type of vaccine carriers that are being explored in our laboratory, through simple linkers that do not have ill influence on the immunological properties of the resultant glycoconjugates. This synthesis is highlighted by combined application of different glycosylation methods to effect the assembly of specific glycosidic linkages.

The synthesis of 5 commenced with the development of a new and efficient synthetic route for 12 and 15 (FIG. 9), using 10 as the common intermediate. In both syntheses, a key step was the tin complex directed regioselective alkylation to give 3-O-alkylated products 11 and 13. Benzoylation of 11 readily afforded glycosyl donor 12. On the other hand, benzylation of 13 followed by oxidative hydrolysis of the thioglycoside in 14 and trichloroacetimidation of the resultant hemiacetal gave glycosyl donor 12 (α:β 12:1) in an excellent overall yield (42%) from 10.

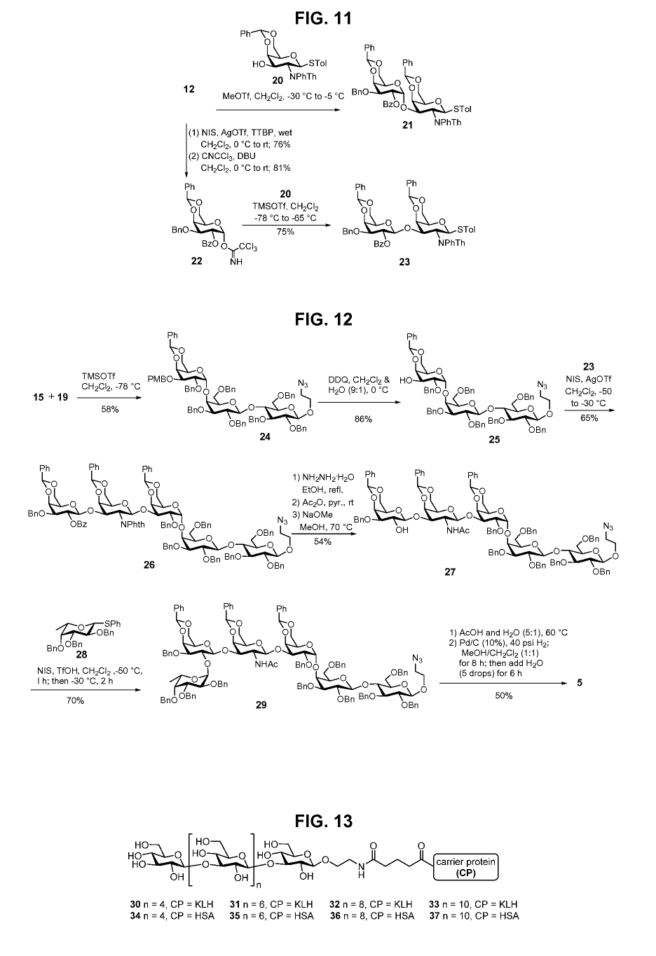

The synthesis of the disaccharide building block 19 (FIG. 11) started from lactose which was first converted into 16 according to a literature procedure. Selective protection of the cis 4′-O— and 6′-O-positions in 16 with the benzylidene ring was carried out successfully by treating 16 with benzaldehyde dimethyl acetal and camphor sulfonic acid to afford 17 in a 74% yield. Perbenzylation of the free hydroxyl groups in 17 was followed by regioselective reductive ring opening of the 4′:6′-O-benzylidene acetal in the resultant 18 to expose the 4′-OH and offer the desired building block 19 smoothly.

To construct the disaccharide block 23, we conducted the glycosylation of 20 with 12 at −5° C. in dichloromethane using methyl trifluoromethanesulfonate (MeOTf) as the promoter. However, it gave the unwanted α-disaccharide 18 as the predominant product (α:β 9:1). A potential explanation for this result was that the presence of benzylidene rings in the donor and acceptor somehow decreased their reactivities to facilitate S_{N}2 type of reaction. To deal with the problem, thioglycoside 12 was converted into the more reactive tricholoroacetamediate 22. Glycosylation of 20 with 22 proceeded smoothly in the presence of trimethyl trifluoromethanesulfonate (TMSOTf) to give the desired β-disaccharide 23 (J_{H-1′,H-2′}=8.1 Hz) as the major product (α:β 1:10) in a good yield (75%). Consequently, 23 was used as a glycosyl donor for the assembly of the target molecule by a [3+2+1] strategy. See FIG. 12.

Next was the installation of Gal III α-linked to Gal II (FIG. 12), which was one of the major challenges in the synthesis of globo H antigen, because in general it is relatively difficult to create the cis α-galactosidic linkage and the galactose axial 4-OH shows relatively low nucleophilicity. To cope with this issue, in addition to using the nonparticipating Bn group for 2-O-protection in 15, also executed is the glycosylation reaction employing a unique experimental procedure of reversed addition, i.e., slowly adding donor 15 to the solution of acceptor 19 and promoter TMSOTf at −70° C. The reaction afforded the desired α-trisaccharide 24 (J_{H-1″,H-2″}=3.2 Hz) in a good yield (58%) and excellent stereoselectivity (α:β 15:1). Selective removal of the 3″-O-PMB group in 24 with DDQ gave trisaccharide 25 as a glycosyl acceptor in an 86% yield.

The coupling reaction between 25 and 23 was accomplished smoothly in CH₂Cl₂ at −30° C. with NIS and AgOTf as promoters. The reaction was stereospecific to generate the β-anomer 26 only (J_{H-1′″,H-2′″}=7.8 Hz). Refluxing 26 with hydrazine hydrate (NH₂NH₂.H₂O) in ethanol removed the Phth and Bz groups smoothly and cleanly (monitored by TLC and MS). The freed amino group and hydroxyl group were acetylated under routine conditions, which was followed by selective removal of the 2″″-O-acetyl group with sodium methoxide in methanol to give 27 as a glycosyl acceptor. Finally, fucosylation of 27 with thioglycoside 28 using NIS and TfOH as promoters resulted in stereospecific formation of the desired hexasaccharide 29 (J_{H-Fuc-1,2}=3.7 Hz) in a good yield (70%). Consequently, we used a two-step protocol for the global deprotection, including the removal of all benzylidene groups in acetic acid and water (5:1) at 60° C. and then hydrogenolysis to remove all of the Bn groups with concomitant reduction of the azido group to a free primary amine, to yield the target molecule 5.

A convergent and highly efficient [3+2+1] strategy was developed for the synthesis of a derivative of the globo H antigen. Optimal conditions were established for generating the glycosidic linkages to achieve efficient synthesis. As a consequence, all of the glycosylation reactions offered good to excellent yields and outstanding stereoselectivity, including the reactions to install the rather challenging cis α-linked D-galactose and L-fucose. Eventually, the target molecule 5 was prepared from a galactose derivative 10 in 11 steps and a 2.6% overall yield, which represented the longest linear synthetic sequence. The good overall yield of the current synthesis would make it feasible to prepare the title compound in relatively large quantities. Moreover, the target molecule 5 carried a free amino group at the glycan reducing end that can be selectively elaborated in the presence of free hydroxyl groups. It would facilitate regioselective conjugation of 5 with other molecules, thus it can be useful for various biological studies and applications.

It is envisioned that in view of the foregoing, a completely synthetic, self-adjuvanting vaccine may be generated by synthesizing an MPLA derivative according to the teachings of U.S. Pat. No. 8,809,285 and using a linker as described herein to conjugate the MPLA derivative to a carbohydrate according to one of the above synthetic TACA molecules. However, the scope of the present invention is inclusive of both synthetic and naturally-derived TACAs, including globo H. Such vaccines will be useful for treatment or prevention of cancers.

### Carbohydrates for Use in Antifungal Vaccines and Therapies

Overview: Antifungal vaccines have recently engendered considerable excitement for counteracting the resurgence of fungal infections. In this context, β-glucan is an attractive target antigen. Aiming at the development of effective antifungal vaccines based on β-glucan, we designed and synthesized a series of its oligosaccharide analogs and coupled them with a carrier protein, keyhole limpet hemocyanin (KLH), to form new semi-synthetic glycoconjugate vaccines. In this regard, a convergent and effective synthetic strategy using pre-activation-based iterative glycosylation was developed for the designed oligosaccharides. The strategy can be widely useful for rapid construction of large oligo-β-glucans with shorter oligosaccharides as building blocks. KLH conjugates of the synthesized β-glucan hexa-, octa-, deca- and dodecasaccharides were demonstrated to elicit high titers of antigen-specific total and IgG antibodies in mice, suggesting the induction of functional T cell-mediated immunity. Moreover, it was revealed that octa-, deca-, and dodeca-β-glucans were much more immunogenic than the hexamer, while the octamer was the best. The results suggested that the optimal oligosaccharide sequence of β-glucan required for exceptional immunogenicity was a hepta- or octamer and that longer glucans are not necessarily better antigens, a finding that may be of general importance. Most importantly, the octa-β-glucan-KLH conjugate provoked protective immunities against Candida albicans infection in a systemic challenge model in mice, suggesting the great potential of this glycoconjugate as a clinically useful immunoprophylactic antifungal vaccine.

Described herein are: (1) developed a highly convergent and effective method for the synthesis of oligosaccharides of β-glucan with varied chain lengths, (2) coupled them with keyhole limpet hemocyanin (KLH), and (3) evaluated the immunological properties of resulting glycoconjugates and their capability to elicit protective immune responses against C. albicans in mice.

Based on reports that a hexasaccharide of β-glucan was immunogenic and that at least an octa- or nonasaccharide may be required to generate special 3D structures, we prepared and compared are hexa-, octa-, deca- and dodecasaccharides of β-glucan (FIG. 13). They were coupled with KLH to form fungus-related vaccines 30-33. In the meantime, the oligosaccharides were also coupled with HSA to provide fungus-related conjugates 34-37 that were used as capture reagents for detecting β-glucan-specific antibodies by ELISA.

As depicted in FIG. 14, the designed β-glucan oligosaccharides were achieved via pre-activation-based iterative glycosylation with p-toluenethioglycosides as glycosyl donors and disaccharide 42 as a key building block. The synthesis was commenced with the preparation of 38 from D-glucose in four steps and in a 40% overall yield. Treatment of 38 with dibutyltin oxide to furnish the stannylene acetal-directed regioselective 3-O-protection with a 2-naphthylmethyl (NAP) group was followed by 2-O-benzoylation of the resultant 39 to afford thioglycoside donor 40. Here, the NAP group was employed as a temporary protection instead of the common para-methoxybenzyl (PMB) group because the former is more stable to acidic conditions involved in glycosylation reactions, although both groups can be readily removed with DDQ. Removal of the 3-O-NAP group in 40 with DDQ was straightforward to give 41 in an excellent yield (92%). Thereafter, 40 was coupled with 41 via pre-activation glycosylation to get 42. Specifically, glycosyl donor 40 was first activated with p-toluenesulfenyl triflate (p-TolSOTf) that was generated in situ from the reaction between p-toluenesulfenyl chloride (p-TolSCl) and silver triflate (AgOTf) at −78° C. Then, glycosyl acceptor 41 was added to furnish glycosylation, resulting in the desired β-disaccharide 42 (J_{1,2}=7.5 Hz, 90% yield) in a stereospecific manner, due to neighboring group participation. Compound 42 was used as one of the common glycosyl donors for subsequent carbohydrate chain elongation. On the other hand, removal of the NAP group in 42 with DDQ provided 43. A convergent [2+2] glycosylation between 42 and 43 by the same pre-activation protocol yielded tetrasaccharide 44 (86%) as a glycosyl donor for more complex oligosaccharide assembly. Pre-activated glycosylation of 2-azidoethanol with 42, followed by removal of the NAP group with DDQ, afforded 45 (91%), which carried an azido group at the non-reducing end. The azido group would be reduced to form a primary amine later on to enable a selective reaction with the linker and then coupling with carrier proteins. Moreover, since the pre-activation-based glycosylation reaction was clean and high yielding and the donor and acceptor were almost completely consumed, this allowed us to move on to the next step after glycosylation, i.e., removal of the NAP group, without purification of the reaction intermediate. Similarly, pre-activation-based glycosylation of 45 with 42 and then removal of the NAP group produced tetrasaccharide 46. On the basis of 46, the sugar chain was further elongated successfully via pre-activation-based glycosylation to achieve all of the designed β-glucan oligosaccharides. Coupling of 46 with disaccharide 42 and tetrasaccharide 44, followed by selective removal of the NAP group, afforded hexasaccharide 47 and octasaccharide 48, respectively. Subsequently, 48 was coupled with 42 and 44, which was followed by NAP group removal to produce decasaccharide 51 and dodecasaccharide 53. Notably, the glycosylation yields were not significantly affected by the increased size of involved building blocks. All of the synthetic intermediates and final products were fully characterized, proving that the glycosylation reactions were β-specific.

Reagents and conditions for FIG. 14: a) Bu₂SnO, toluene, reflux, 6 h; then 2-naphthylmethyl bromide, CsF, DMF, 70° C., 12 h, 72%; b) BzCl, Et₃N, CH₂Cl₂, rt, 12 h, 96%; c) DDQ, CH₂Cl₂/H₂O (18:1), rt, 8 h, 92% for 41, 95% for 43; d) AgOTf, TTBP, p-TolSCl, CH₂Cl₂, −78° C. to rt, 4 h, 90% for 42, 86% for 44; e) AgOTf, TTBP, p-TolSCl, CH₂Cl₂, −78° C., rt, 4 h; then DDQ, CH₂Cl₂/H₂O (18:1), rt, 8 h, 91% for 45, 90% for 46, 87% for 47, 81% for 48, 80% for 51, 85% for 53; f) Zn, AcOH, CH₂Cl₂, 24 h, rt; then AcOH/H₂O (5:1), 60° C., 24 h; finally NaOH, t-BuOH:H₂O, 40° C., 24 h, 80% for 49, 88% for 50, 85% for 52, 88% for 54.

Eventually, 47, 48, 51 and 53 were fully deprotected in a stepwise manner using the proper solvent or solvent combination for each transformation, in the order of Zn-mediated reduction of the azido group in dichloromethane, acidic cleavage of all benzylidene groups in acetic acid and water (5:1), and finally sodium hydroxide-promoted removal of all benzoate groups in t-butyl alcohol and water (4:1). The final products were purified with a Sephadex-G25 size exclusion column with distilled water as the eluent to afford 49, 50, 52 and 54 as white fluff solids upon lyophilization.

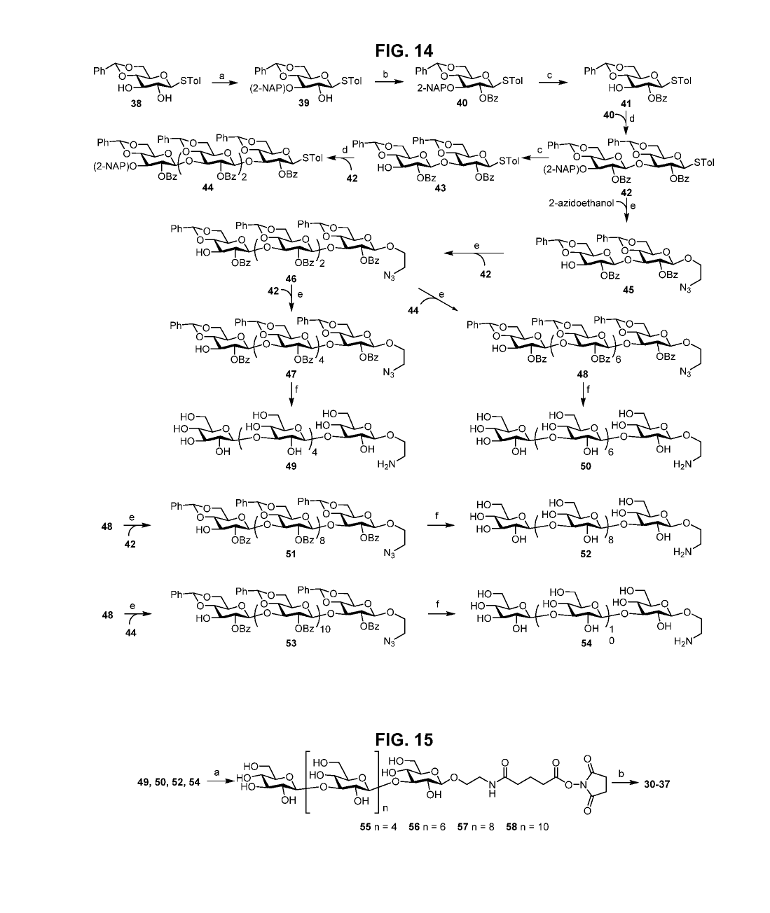

Conjugation of oligosaccharides 49, 50, 52 and 54 with carrier proteins: Free oligosaccharides 49, 50, 52 and 55 were conjugated with carrier proteins KLH and HSA through the bifunctional glutaryl group as mentioned above. A two-step procedure was used to furnish the conjugation (FIG. 15). First, reaction between the free amino group in 49, 50, 52 and 54 and a large excess of active ester disuccinimidal glutarate (DSG) gave the corresponding mono-activated esters 55-58 in quantitative yields. Then, 55-58 were coupled with KLH or HSA in 0.1 M phosphate-buffered saline (PBS) to afford the desired glycoprotein conjugates 30-37, which were purified with a Biogel A0.5 column to remove remaining free sugars. The conjugate-containing fractions were dialyzed against distilled water and lyophilized to give 30-37. Finally, the glucose content of each conjugate was analyzed by the phenol-sulfuric acid method following a reported protocol. The glucose contents of the KLH and HSA conjugates were 7.5-9.1% and 10.5-25.8%, respectively (Table 1), showing that the coupling reactions were efficient and the antigen loading levels were in the desired range for glycoconjugate vaccines. The sugar loadings of HSA conjugates were also confirmed by MALDI-TOF mass spectrometry.

Reagents and conditions for FIG. 15: a) DSG, DMF and PBS buffer (4:1), rt, 4 h; b) KLH or HSA, PBS buffer, rt, 2.5 days.

TABLE 1
Carbohydrate loadings of glycoconjugates 30-37

| | KLH conjugates | | | | HSA conjugates | | | |
|---|---|---|---|---|---|---|---|---|
| | Sample | | | | | | | |
| | 30 | 31 | 32 | 33 | 34 | 35 | 36 | 37 |
| Loading (%) | 8.3 | 7.8 | 7.5 | 9.1 | 10.5 | 11.0 | 14.3 | 25.8 |

Immunological Studies of Glycoconjugate Vaccines 30-33.

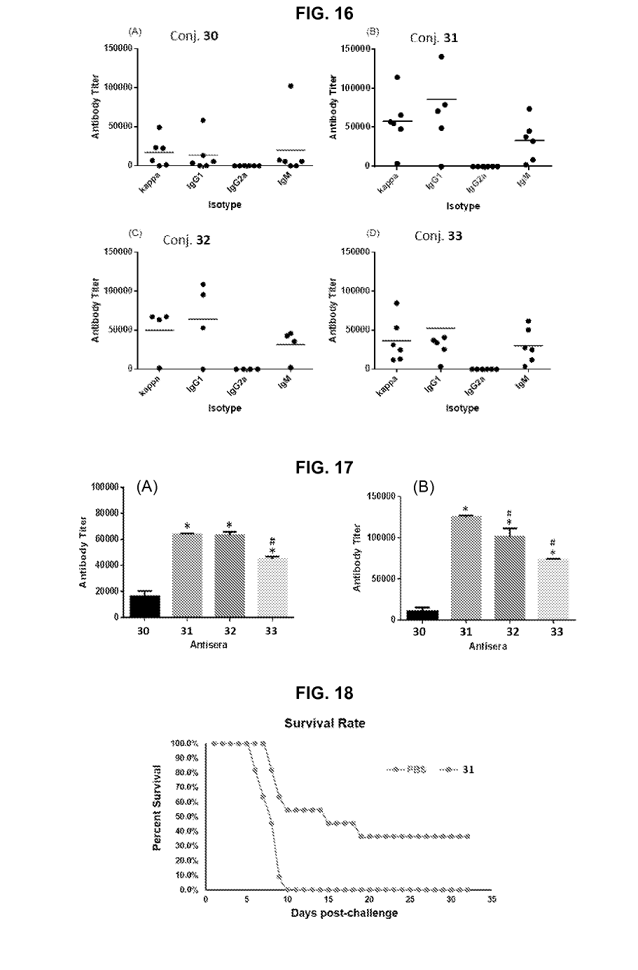

The immunological properties of KLH conjugates 30-33 were investigated in female C57BL/6J mice. For this purpose, each conjugate was thoroughly mixed with Titermax Gold adjuvant, and the resulting emulsion was then injected intramuscularly (i.m.) into mice. Following the initial immunization, mice were boosted 4 times on days 14, 21, 28 and 38 by subcutaneous (s.c.) injection of the same vaccine emulsion. Blood samples of each mouse were collected through the leg veins prior to the initial immunization on day 0 and after immunizations on days 27, 38 and 48. Antisera were obtained from clotted blood samples and were stored at −80° C. before use. ELISA using the corresponding HSA conjugates as capture reagents for plate coating was employed to determine antibody titers, which reflected the elicited immune responses. Antibody titers were defined as the dilution number yielding an OD value of 0.2, and the results are shown in FIG. 16.

FIG. 16 illustrates ELISA results of the day 48 antisera obtained with 30 (A), 31 (B), 32 (C) and 33 (D) combined with Titermax Gold adjuvant, respectively. The titers of corresponding antigen-specific antibodies are displayed. Each dot represents the antibody titer of an individual mouse, and the black bar shows the average titer.

All of the KLH conjugates 30-33 elicited high titers of antigen-specific total (kappa) antibodies, indicating strong immune responses. More importantly, high titers of IgG1 antibodies were observed for all glycoconjugates, suggesting memorable T cell-dependent immunities. IgG1 antibody is usually considered as the protective antibody isotype, thus these conjugates elicited protective immune responses and have great potential for being developed into clinically functional vaccines against fungal infections.

The above immunological results revealed that, overall, conjugates 31-33 induced significantly higher titers of both total (anti-kappa) and IgG1 antibodies than 1 (P<<0.01, FIG. 17), indicating that 31-33 were much more immunogenic and provoked much stronger immune responses in mice than 30. Further analysis of the immune responses showed that the IgG1 antibody titer induced by 31 was significantly higher than that induced by 32 and 33 (P<0.01, FIG. 17B) as well. Although the total antibody titer for 2 was also slightly higher than that for 32 and 33 (FIG. 17A), this difference was less significant (P>0.05 and <0.01, respectively). There are several factors that may affect the immune response to a glycoconjugate, such as carbohydrate loading, conjugation method, immunization protocol, and carbohydrate antigen structure. The carbohydrate loadings of 30-33 were very similar, and their conjugation method and immunization protocol were identical. Therefore, the different immunological properties for these glycoconjugates were because of their different carbohydrate structures, and among the oligosaccharides investigated here octa-β-glucan seemed to be the most immunogenic and the most promising antigen for vaccine development.

FIG. 17 is a comparison of the average antibody titers of corresponding antigen-specific (A) total (anti-kappa) antibodies and (B) IgG1 antibodies in the day 48 pooled antisera of mice immunized with conjugates 30-33, respectively. Each error bar is the standard deviations for three parallel experiments. * P<<0.01 as compared to 30; # P<0.05 as compared to 31.

Protection Against Fungal Infection in Mouse:

To ultimately prove the efficacy of the new glycoconjugate vaccine to protect against fungal infections, conjugate 31 that elicited the strongest immune responses in above studies was evaluated in a fungal challenge experiment in mice. The fungus used was C. albicans (strain SC5314), one of the most common and important pathogenic fungi in clinic. In this experiment, each group of 11 mice were immunized with 31 or PBS (the control group) 4 times on days 1, 14, 21, and 28 according to above-mentioned protocols. On day 38, a pre-determined lethal dose of C. albicans (7.5×10⁵ cells/mouse in 200 μL PBS) was given by i.v. injection to each mouse. The responses of these mice were observed under normal feeding and care conditions. As shown in FIG. 18, mice in the control group started to die of infection on day 6 after the fungal injection, and all died within 4 days (on day 10). In comparison, mice in the 31-immunized group did not have fatal incident until day 8, and on day 14 the animal survival rate was about 55%. At the end of this experiment (on day 32), there were still four mice (about 34%) in the immunized group unaffected, suggesting complete protection of these mice from C. albicans infection. These results proved that glycoconjugate 31 could elicit protective immunity in mice against lethal systemic challenge with C. albicans.

FIG. 18 shows survival time of mice immunized with antifungal conjugate 31 (top line) compared with mice immunized with PBS (bottom line) after i.v. injection of C. albicans (7.5×10⁵ cells per mouse and 11 mice per group).

In summary, a series of β-glucan oligosaccharides were synthesized and coupled with KLH to generate glycoconjugates that contained structurally well-defined carbohydrate antigens. These glycoconjugates were shown to elicit robust T cell-dependent and protective immune responses in mice, which helped identify the promising antifungal vaccines. This work is distinguished from previous studies in the area in several aspects. First, a highly convergent, effective and potentially broadly applicable strategy was developed for the synthesis of structurally well-defined β-glucans. Large oligosaccharides could be rapidly assembled from short oligosaccharide segments by the pre-activation-based glycosylation protocol that had significantly reduced the number of steps for anomeric manipulation. Furthermore, with the help of neighboring group participation, all of the glycosylations were highly stereoselective to create the desired β-anomer. Therefore, this synthetic strategy can be widely applicable to larger and more complex β-glucan derivatives via [n+n] or [n+(n+1)] glycosylations.

Second, the synthesized oligosaccharides had a reactive amino group at their reducing ends, enabling their effective coupling with carrier proteins, such as KLH, through a bifunctional linker. Although a number of 3-glucan oligosaccharides have been synthetized previously, only a few have been conjugated with a carrier protein and investigated as vaccines. On the other hand, conjugate vaccines currently employed for biological studies are typically made of heterogeneous natural β-glucans or oligosaccharides derived from natural β-glucans.

Third, immunological studies of glycoconjugate vaccines 30-33 revealed that while all of them could elicit robust T cell-dependent immune responses, octa-, deca-, and dodeca-β-glucans were much more immunogenic than hexa-β-glucan, which was different from the literature results. These results suggest that at least an octamer is necessary for oligo-β-glucans as optimal antigens for elicitation of functional immune responses. However, this does not necessarily mean that the longer the better for an oligosaccharide antigen. As a result, an octa- or nona-β-glucan was identified as the most promising antigen for designing and developing β-glucan-based antifungal vaccines.

Finally and most importantly, we have demonstrated in a mouse model that the conjugate of KLH and octa-β-glucan, namely antifungal conjugate 31, could elicit protective immune responses against the deadly pathogen C. albicans. This result is highly relevant to clinic application. Therefore, this work has paved the foundation for developing an effective and clinically useful antifungal vaccine.

Not only are linear molecules useful in antifungal applications. According to another aspect of the present invention, the use and synthesis of branched carbohydrate antigens are also described.

Branched β-glucan oligosaccharides are prepared by a highly convergent and efficient strategy. The strategy was highlighted by assembling the title compounds via preactivation-based glycosylation with thioglycosides as glycosyl donors. It was used to successfully prepare β-glucan oligosaccharides that had a β-1,3-linked nonaglucan backbone with β-1,6-glucotetraose, β-1,3-glucodiose and β-1,3-glucotetraose branches at the 6-O-position of the nonaglucan central sugar unit. The strategy can be generally useful for the synthesis of more complex structures.

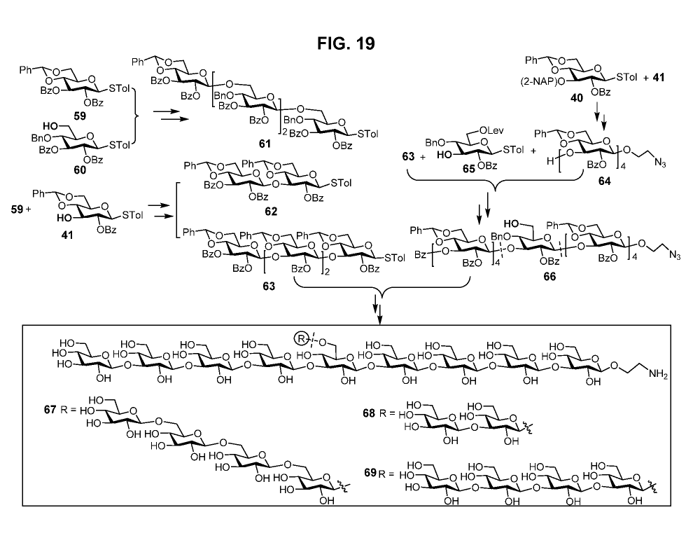

FIG. 19 shows the synthetic targets of branched β-glucan oligosaccharides 67-69 and the highly convergent and efficient strategy for their synthesis relying on preactivation-based iterative glycosylation with thioglycosides as glycosyl donors. The oligosaccharides had a β-1,3-linked nonaglucan backbone with branches, including β-1,6-glucotetraose (67), β-1,3-glucodiose (68) and β-1,3-glucotetraose (69), attached to the 6-O-position of the central sugar unit of the nona-β-glucan. They were supposed to span different structural properties and immunological determinant epitopes of natural β-glucans. Moreover, we attached a free aminoethyl group to the reducing end of these oligosaccharides to facilitate their conjugation with various biomolecules and tags, such as carrier proteins, to be useful for biological studies and conjugate vaccine development.

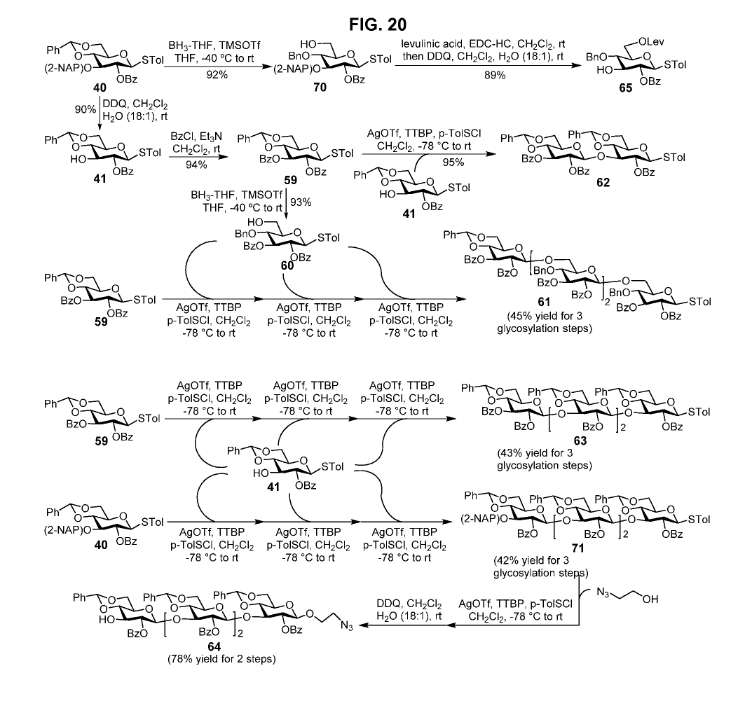

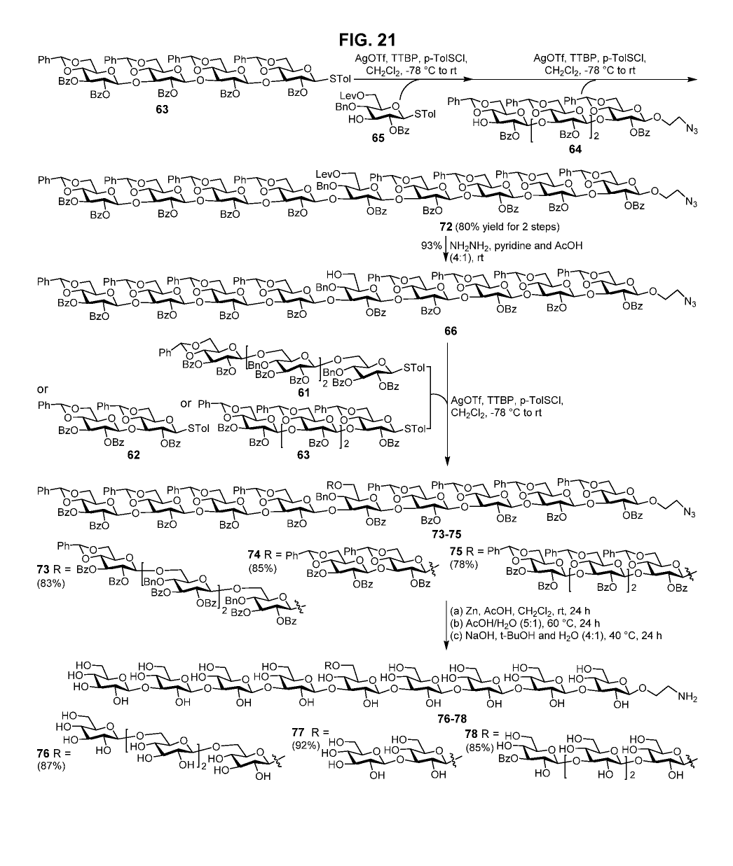

Our synthesis (FIG. 20) commenced with the preparation of 40. Regioselective removal of the NAP group at the 3-O-position in 40 with DDQ, followed by 3-O-benzoylation and then regioselective reductive ring opening of the benzylidene acetal in 59 using BH₃.THF and TMSOTf, afforded 60. On the other hand, reductive ring opening of the benzylidene acetal in 40, followed by protection of the exposed 6-O-position with a levulinoyl (Lev) group through reaction with levulinic acid and EDC.HCl and then deprotection of the 3-O-position with DDQ, produced 65. Consequently, all of the required monosaccharide building blocks were readily synthesized from 40 with excellent overall yields.

For the synthesis of disaccharide block 62, the preactivation glycosylation protocol was applied. First, glycosyl donor 59 was treated with the promoter p-TolSOTf (1.0 equiv.), which was formed in situ from the reaction of pp-TolSCl with AgOTf, at −78° C. for 10 min, and then glycosyl acceptor 41 (0.9 equiv.) was added for glycosylation. The reaction was β-specific to accomplish 62 in a 95% yield. Starting from 59, tetrasaccharide 61 and 63 were prepared through preactivation-based iterative one-pot glycosylation using 60 and 41 as glycosyl donors, respectively (FIG. 20). Preactivation of the thioglycosyl donors with p-TolSOTf was carried out at −78° C. for 10 min in a mixture of dichloromethane and acetonitrile. After the donor was completely consumed (in ca. 5 min at −78° C., shown by TLC), 0.9 equivalent of an acceptor was added together with 2,4,6-tri-t-butylpyrimidine (TTBP), which was used to neutralize trifluoromethanesulfonic acid formed from the glycosylation reaction. It was then warmed to room temperature for ca. 20 min to guarantee complete consumption of the accepter as indicated by TLC. Then, the mixture was cooled to −78° C. to perform another round of preactivation and glycosylation by the same protocol. After the third round of glycosylation and then workup, 61 and 63 were obtained in 45% and 43% isolated yields, respectively. Similarly, tetrasaccharide 71 was prepared from 40 and 41 via iterative one-pot glycosylation in an overall yield of 42%, suggesting that each glycosylation step gave an average of more than 75% yield and that the overall yields did not show a significant difference for β-1,6- and R-1,3-linked tetrasaccharides. Eventually, 71 was transformed into building block 64 upon glycosylation with 2-azidoethanol in the presence of p-TolSCl/AgOTf and removal of the 2-NAP protecting group with DDQ. All of the glycosylation reactions were β-specific, confirmed by the ¹H NMR spectra of 61, 62, 63 and 64 with the coupling constants in the range of 6.2-10.1 Hz for all anomeric protons.

The preactivation-based one-pot glycosylation protocol was also used to prepare protected nonasaccharide 72 from 63, 65 and 64 (FIG. 21). Delightfully, these reactions gave an excellent overall yield (80%), despite that they involved rather complex glycosyl donors and acceptors. Thereafter, the Lev group at the 6-O-position of the central sugar residue in 72 was selectively removed with hydrazine to accomplish 66. Glycosylation of 66 with 61, 62 and 63 in the presence of p-TolSCl/AgOTf to install the branches was smooth and gave full protected 73, 74 and 75, respectively, in very good yields. Global deprotection of 73-75 was performed by a stepwise, one-pot protocol to deal with the solubility problem of various partially deprotected reaction intermediates. Thus, 73-75 were first treated with Zn and acetic acid in dichloromethane to reduce the azide group. After filtration to remove solids and concentration to remove solvents, the crude product was dissolved in acetic acid and water (5:1) and was heated at 60° C. to remove all of the benzylidene groups. Finally, the benzoyl groups were removed with sodium hydroxide in tert-butanol and water (4:1) to afford the desired products 76, 77 and 78 that were purified with a Sephadex-G25 size exclusion column.

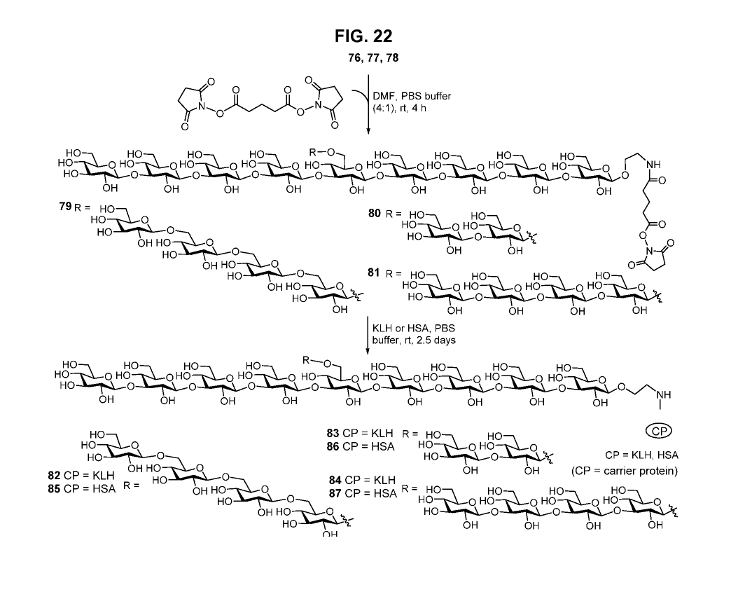

The synthetic β-glucan oligosaccharides 76-78 were then coupled with the keyhole limpet hemocyanin (KLH) to form conjugates 82-84 as vaccines (FIG. 22). Moreover, the human serum albumin (HSA) conjugates 85-87 of the oligo-β-glucans were prepared and used as coating antigens for enzyme-linked immunosorbent assays (ELISA) of carbohydrate antigen-specific antibodies.

Preparation of Glycoconjugates:

Oligosaccharides 76-78 prepared above were coupled with KLH and HSA through a bifunctional glutaryl linker (FIG. 22). The procedure was the same as that described in section 900106]. The carbohydrate loadings of HSA conjugates 85-87 were also assessed with MS (Supporting Information), which were 12.1%, 15.2%, and 15.5% for 85, 86 and 87, respectively, compared to 9.8%, 15.1%, and 13.7% given by the phenol-sulfuric acid method (Table 1). The conjugation of oligo-β-glucans with KLH was verified by SDS-PAGE, which showed the increase in molecular mass of 82-84 compared to native KLH. The results have demonstrated that conjugation reactions between 76-78 and proteins was efficient and the antigen loading levels of 82-87 were in the desirable range.

Immunologic study of glycoconjugates. The immunologic properties of 82-84 as vaccines were evaluated in female C57BL/6J mice by the same methods and protocols described in section [00109].

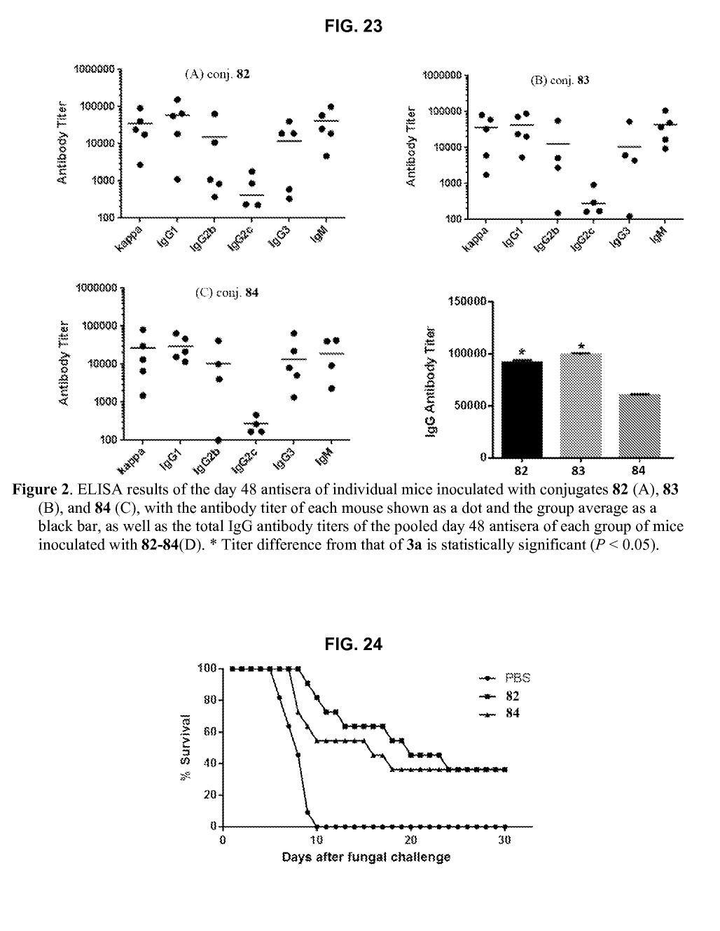

ELISA results in (FIG. 23) suggested that all of the conjugates 82-84 elicited high titers of antigen-specific total (kappa) antibodies (FIG. 23A-C) and strong immune responses. Individual antibody isotype analysis revealed the production of high levels of IgM, IgG1, IgG2b, and IgG3 antibodies, as well as a low level of IgG2c antibody. Production of IgG antibodies, especially IgG1 and IgG2b types, indicated T cell-mediated cellular immunity. Moreover, IgG1 and IgG2b antibodies were shown to have high antigen binding affinities and are considered the protective antibody isotypes. Therefore, we believed that 82-84 elicited memorable and protective T cell-mediated immunities desirable for prophylactic vaccines.

It was also observed that 82 and 83, which had antigens with a β-1,6-linked tetraglucose and β-1,3-linked diglucose branches, elicited similar titers of total IgG antibodies, 91,866 and 99,196 respectively, that were higher than the total IgG antibody titer of 84 (60,219) with a β-1,3-linked tetraglucose branch (FIG. 23D). These results indicated that 82 and 83 were more immunogenic than 84. Nevertheless, 84 induced robust and consistent immune responses in all tested mice.

Binding assays between natural glucans or fungal cells and antisera. To probe whether antibodies elicited by 82-84 could recognize natural β-glucans, we analyzed the influence of Lam, a β-glucan carrying sporadic branches at the main chain 6-O-positions, on the binding between synthetic oligo-β-glucans and anti-82-84 sera. Antisera (1:900 dilution) were mixed with various concentrations (0, 0.01, 0.1, 1, 10, 100, and 200 μg/mL) of Lam and then applied to ELISA with HSA conjugates 85-87 as capture antigens. Antibody binding to Lam was shown by the decrease in the number of antibodies bound to 85-87 on the plates due to Lam-caused competitive binding inhibition, which was calculated according to the equation presented in the experimental section. Our results showed that Lam indeed had inhibition on antibody binding to 85-87 in a concentration-dependent manner, and at 200 μg/mL, the inhibition was >90% in all three cases. The 50% inhibition concentrations (IC50) were about 5 μg/mL. Evidently, the antibodies elicited by 82-84 could recognize and bind to Lam.

C. albicans (HKCA) cell-antiserum binding was studied by immunofluorescence (IF) assay. Heat-killed HKCA cell was treated with BSA blocking buffer to mask potentially nonspecific protein binding sites on the cell surface and incubated with pooled antisera. The cell was stained with a FITC-labeled goat anti-mouse kappa antibody and examined with microscope. The results showed that compared to the negative control, both the fungal particles and hyphal cells were uniformly IF stained, indicating the strong binding of antisera to HKCA cell.

Protection against fungal infection: To validate the new conjugates as antifungal vaccines, 82 and 84, whose carbohydrate antigens had the same length of side chains but different glycosyl linkages, were evaluated for protection against fungal infections using a mouse challenge model. The fungal cell used was Candida albicans (strain SC5314), one of the most common pathogenic fungi in clinic. Each group of 11 mice were immunized 4 times with 82, 84 or PBS buffer (negative control). After positive immune responses were affirmed, a pre-determined lethal dose of C. albicans cells (7.5×105 cells/mouse in 200 μL PBS) was i.v. injected in each mouse. The mice were monitored, and their survival time and rate are shown in FIG. 24.

As depicted in the FIG. 24, mice in the control group started to die on day 5 after C. albicans challenge, and all died of fungal infection in 10 days. No death occurred to the mice immunized with 82 and 84 until days 8 and 7, respectively, and the animal survival rate was about 82% for 82 and 55% for 84 on day 10. At the end of the experiment (day 30 after fungal challenge), there were still 37% of mice survived in groups immunized with 82 and 84, suggesting potentially complete protection of the mice from C. albicans challenge. The results unambiguously confirmed that conjugates 82 and 84 elicited functional immunities that could effectively protect mice from C. albicans-caused infection. Moreover, 82 provided better protection against C. albicans than 84 at the onset of infection, which was consistent with the discovery that 82 elicited stronger immune response than 84. However, these two vaccines had similar long-term protection against C. albicans infection.

The KLH conjugates of all three synthetic branched oligo-β-glucans elicited strong T cell-mediated immunity highly desirable for prophylactic vaccines. The results obtained here and in a previous study suggested that 82-84 elicited similar pattern and strength of immune responses as the KLH conjugate of an optimized linear oligo-β-glucan, i.e., β-octaglucan. Thus, branched oligo-β-glucans should be at least as similarly promising antigens as linear oligo-β-glucans. It was also shown that antibodies induced by 82-84 could recognize and bind to natural β-glucans and fungal cells. Most importantly, 82 and 84 elicited protective immunities against systemically administered lethal C. albicans in mice. The immunologic results of 82-84 were similar to that of Lam-CRM197 conjugate. Our studies have thus proved that branched oligo-β-glucans, after conjugation with KLH, and more favorably other carrier proteins such as TT, DT, and CRM₁₉₇, can be developed into functional antifungal vaccines.

Our studies further indicated that the number and/or density of side chains in branched oligo-β-glucans is important for their immunologic property. It seemed that to elicit protective immunity, branched oligo-β-glucans needed to carry fewer but longer than monosaccharide branches.

Although 82 provoked stronger immune responses than 84, the two conjugates had similar long-term protection against C. albicans. Moreover, the long-term protection rate for 82 and 84 (both 37%) was only slightly higher than that (34%) of the KLH conjugate of linear β-octaglucan. These results suggested that so long as the conjugates provoked robust T cell-mediated immunity, they would be able to provide protection against C. albicans, even if they had different antibody titers. We expect that if more immunogenic carrier proteins, such as CRM197 or tetanus toxoid, are utilized to conjugate with the oligo-β-glucans, more potent vaccines and better protection results against fungi may be obtained.

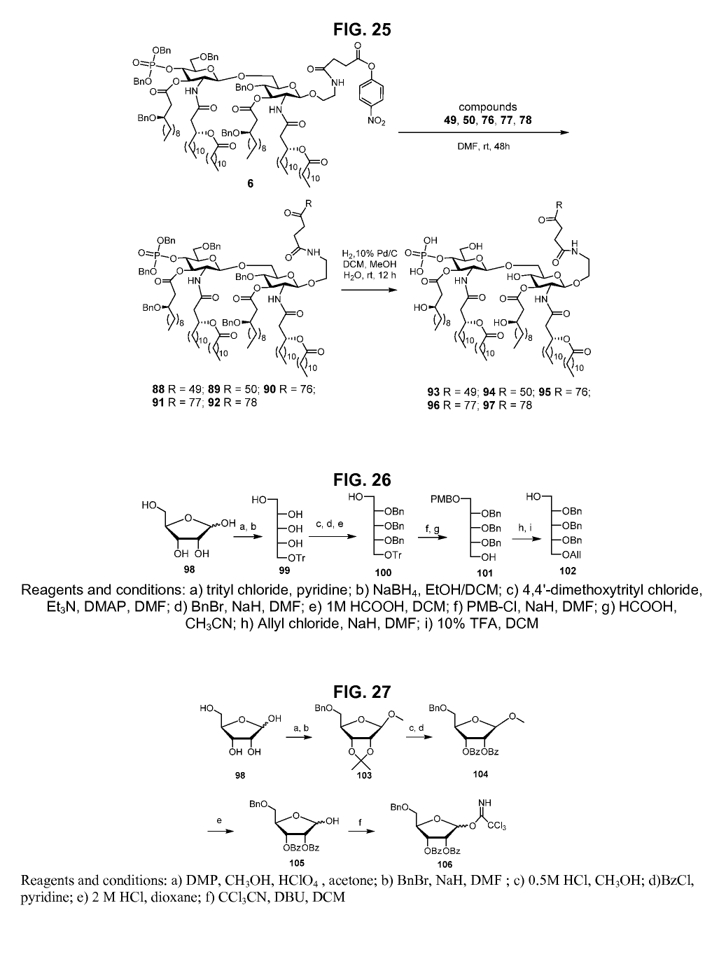

The synthetic linear and branched oligo-β-glucans or β-glucan oligosaccharides 49, 50, 76, 77, and 78 were also coupled with MPLA (FIG. 25). Evaluation of the resultant conjugates 93-97 without using an external adjuvant gave similar results as that of the KLH conjugates in combination with adjuvants. These studies demonstrated that the MPLA conjugates of synthetic β-glucan oligosaccharides are also promising antifungal vaccines.

It is envisioned that in view of the foregoing, a completely synthetic, self-adjuvanting vaccine may be generated by synthesizing an MPLA derivative according to the teachings of U.S. Pat. No. 8,809,285 and using a linker as described herein to conjugate the MPLA derivative to a carbohydrate according to one of the above synthetic carbohydrate molecules, such as a linear or branched β-glucan. Such a vaccine will be useful for treatment or prevention of fungal infections and diseases, including those caused by C. albicans.

### Carbohydrates for Use in Antibacterial Vaccines and Therapies

The use of any polysaccharide conjugate vaccine below is contemplated for use in treatment or prevention of a bacterial infection, particularly in the context of a self-adjuvanting vaccine.

It has been well known for many years that antibodies to the capsular polysaccharide PRP (shown below) of Haemophilus influenza type b (Hib), a polymer of repeating ribosyl ribitol phosphate (RRP) units, are protective against a serious disease caused by bacterial meningitis and other invasive bacterial disease. Thus, we investigated the use of PRP oligosaccharides for the development of fully synthetic glycoconjugate vaccines against Hib using MPLA as carrier molecule.

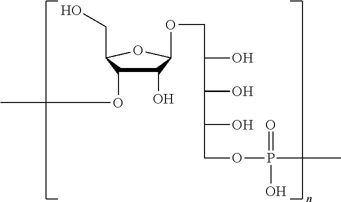

First, we developed an efficient method for the synthesis of structurally well-defined PRP oligosaccharides. Even though there are several literature reports for the synthesis of the intermediate alcohol 102, our new synthesis is simpler and more efficient (FIG. 26). D-ribose was first converted to 5-O-trityl-D-ribose, which was subsequently reduced with NaBH₄ to afford tetraol 99 according to the literature procedure. Treatment of 99 with 4,4′-dimethoxytrityl chloride and catalytic amount of DMAP in DMF selectively gave 2,3,4-triol intermediate, which was subsequently subjected to benzylation, followed by deprotection of dimethoxytrityl groups in 1 M formic acid in dichloromethane to give alcohol 100. Alcohol 100 was then protected as its PMB-ethers by treatment with NaH and PMB-Cl in DMF. Trityl group deprotection using formic acid in acetonitrile then furnished alcohol 101. Allylation of alcohol 101 and removal of para-methoxybenzyl group using 10% trifluoroacetic acid in dichloromethane finally gave ribitol 102 in seven steps and 53% overall yield from tetraol 98.

FIG. 27 shows the synthesis of compound 106. D-ribose was first converted to 104 following literature procedure in four steps. Treatment of 104 with 2M HCl in dioxane gave hemiacetal 105. Trichloroacetimidate 106 was subsequently prepared from 105 by reaction with trichloroacetonitrile and catalytic amount of DBU in dichloromethane.

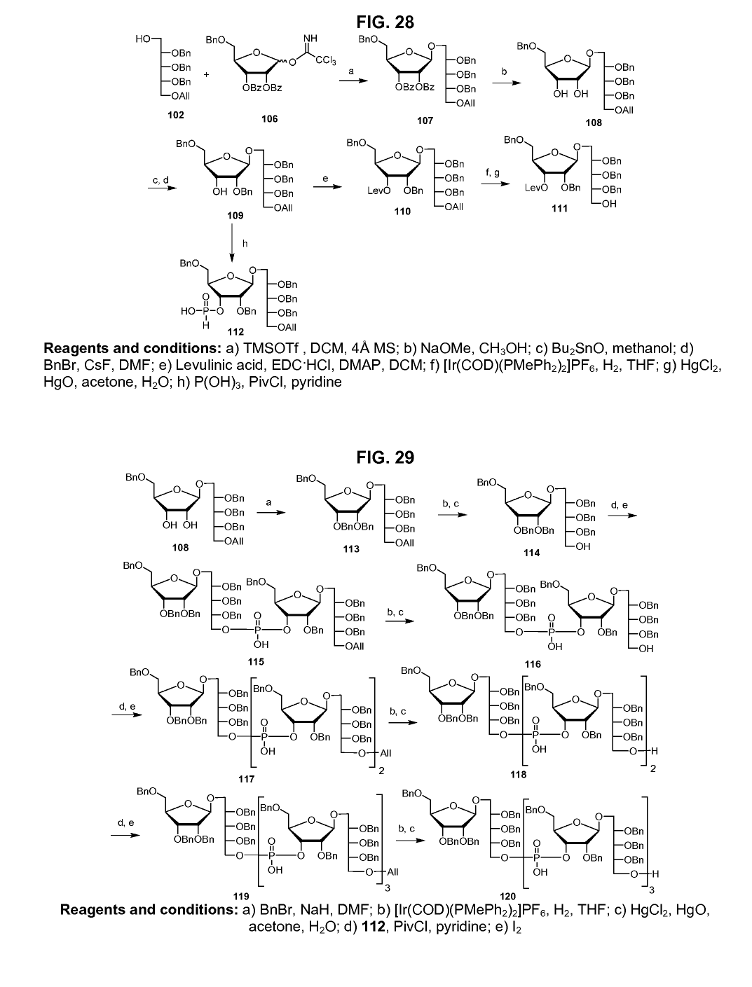

FIG. 28 shows the syntheses of compounds 111 and 112. After comparison with several monomers of the ribosyl-ribitol unit used in previously reported syntheses of PRP fragments, compound 107 was chosen for this work, as it offers several advantages over the others. The glycosylation of 102 with imidate 106 proceeded smoothly in presence of catalytic TMSOTf in dichloromethane at 0° C. to produce 107 in high yield (95%). Furthermore, diol monomer 108 obtained by debenzoylation (using sodium methoxide) was subjected to stannylene acetal-directed regioselective protection of ribose 2-O-position with benzyl ethers using CsF/BnBr in DMF to give the key intermediate 109 in higher yield (70%) than the triol monomer. Compound 109 was treated with levulinic acid, EDC.HCl and catalytic amount of DMAP in dichloromethane to furnish ester 110, which further confirmed the structure of 109. The isomerization of allyl ether 110 to the corresponding vinyl ether was completed by Ir-catalyst, followed by treatment with HgCl₂ and HgO in acetone-water (9:1) to form alcohol 111. Treatment of 109 with phosphonic acid in dry pyridine afforded the H-phosphoate 112, which could be combined with another ribosyl-ribitol unit at the 5-position.

FIG. 29 shows the synthesis of compound 120. Benzylaton of diol 108 gave fully protected 113, which was further subjected to deallylation to give the terminal monomer alcohol 114. Phosphorylation of 114 in pyridine with 112 afforded the dimer 115, which upon subsequent deallylation generated alcohol 116. Elongation process by repetition of the phosphorylation and deallylation sequence gave the trimer alcohol 118 and tetramer alcohol 120 in high yields.

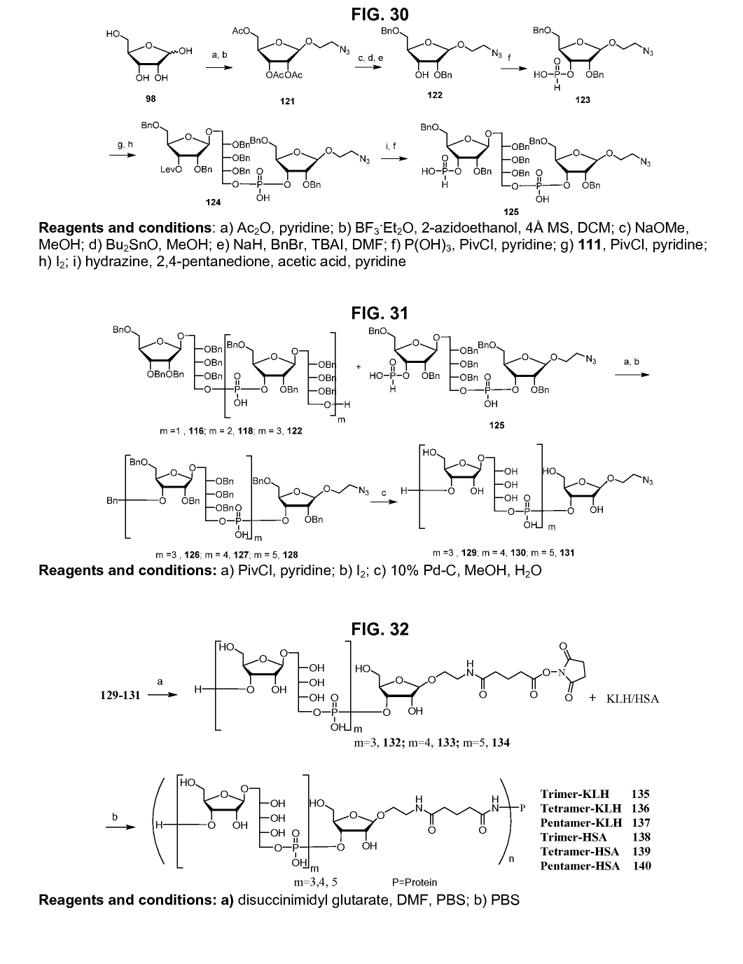

FIG. 30 shows the synthesis of compound 125. Reaction of D-ribose 98 with acetic anhydride in pyridine furnished ribose tetraacetate. Treatment of the tetra-acetate compound with 2-azidoethanol and BF₃.Et₂O in dry dichloromethane afforded 121 as only a isomer, which was verified by comparison with the ¹³C NMR-shift from the known literature data for methyl ribofuranosides. Saponification of 121 with catalytic sodium methoxide in methanol gave triol intermediate. Selective dibenzyl protection at c-2 and c-5 hydroxy groups was carried out by treating the triol intermediate with dibutyltin oxide in refluxing methanol, followed by addition of sodium hydride, tetrabutylammonium iodide and benzyl bromide in DMF. Compound 123 was prepared from 122 following the same procedure described for 112. Compound 41 was then subjected to phosphorylation with 111 to obtain 124. Removal of levulinyl ester by treatment with hydrazine in pyridine-acetic acid buffer afforded alcohol intermediate, which was then treated with phosphonic acid in dry pyridine to give linker moiety 125.

FIG. 31 shows the synthesis of trimer, tetramer, and pentamer of the polysaccharide repeating unit. Construction of the phosphodiester linkages between 125 and 116, 118 or 122 under the same coupling conditions with 112 and 114 proceeded smoothly to furnish the fully protected target trimer 126, tetramer 127 and pentamer 128 in excellent yields, which were readily deprotected by Pd-catalyzed hydrogenolysis in the mixture solution of methanol and water to give the desired amino-oligosaccharides 129, 130 and 131 respectively as white triethylammonium salt in quantitative yield.

FIG. 32 shows the synthesis of glycoconjugates from the oligosaccharides 129, 130 and 131. Treatment of the amino-oligosaccharides 129, 130 and 131 with disuccinimidal glutarate in DMF:PBS buffer at room temperature gave corresponding activated esters 132, 133, and 134 in quantitative yields. Final conjugation was achieved by coupling the activated oligosaccharides with HSA/KLH in PBS buffer at room temperature for 3 days. The reaction mixtures were purified with Biogel A 0.5 column, dialyzed against deionized water, and then lyophilized to afford the desirable glycoconjugates 135-140 as white solids.

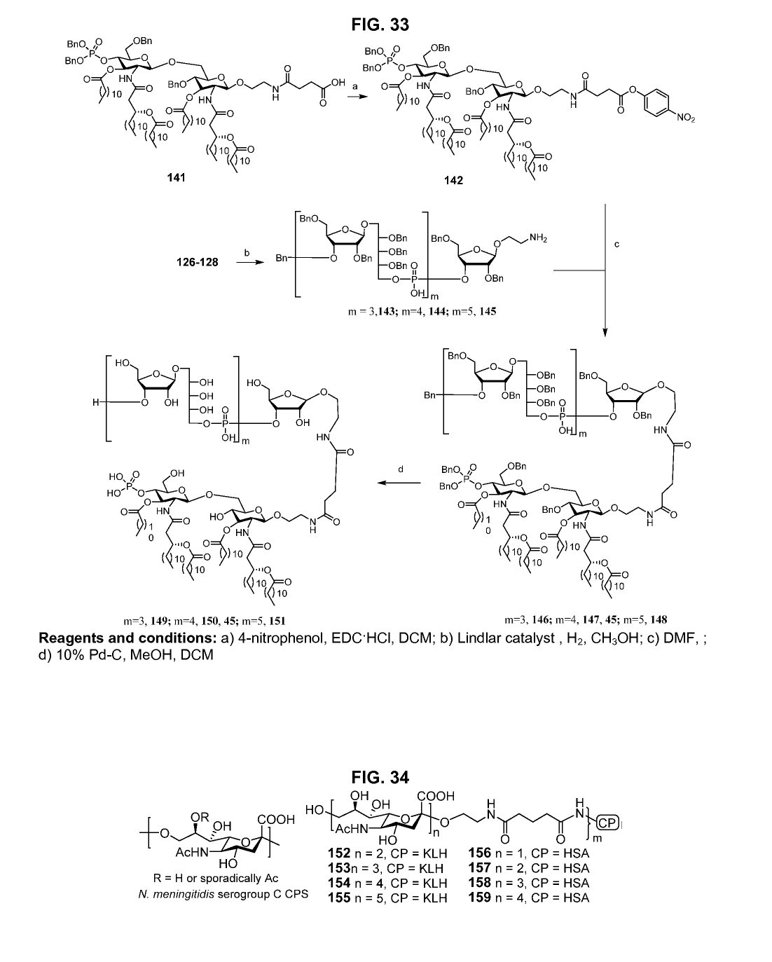

FIG. 33 shows the synthesis of lipid A glycoconjugates from the oligosaccharides 126-128. Selective reduction of azide group in 126-128 to amine group was carried out using lindlar-catalyzed hydrogenolysis in methanol to give the fully protected amino-oligosaccharides 143-145, which were directly used to react with the activated ester 142 to give corresponding lipid conjugates 146-148. The lipid conjugates were then purified by preparative TLC plate and subsequently passing through sephadex LH 20 column. Purified lipid conjugates were then subjected to Pd-catalyzed hydrogenolysis, affording the target compounds 149-151 in quantitative yield.

Both the Hib oligosaccharide-KLH conjugates 135-137 and the Hib oligosaccharide-lipid A conjugates 149-151 illustrated above were evaluated in mice to demonstrate that they could induce strong immune responses. Therefore, they, as well as the oligosaccharide conjugates with other proteins such as TT, DT and VRM₁₉₇ are suitable for use in vaccine compositions. Because of attachment to MPLA, these conjugates constitute self-adjuvanting vaccines.

Treatment and prevention of group C Neisseria meningitis and other diseases caused by bacteria are also contemplated.

Neisseria meningitidis is an important human pathogen and a major cause of bacterial meningitis and sepsis. So far, 13 serogroups of N. meningitidis have been identified and are classified according to the structure of their cell surface capsular polysaccharides (CPSs). In industrialized countries, group C is one of the strains mainly responsible for meningitis epidemics.

For the control of endemic and epidemic meningitis, vaccination is considered an important and effective strategy. For vaccine design, CPSs on the meningococcal cell surface are considered the ideal targets, as they are not only the major and the most exposed but also the most conserved components on bacterial cells. The first CPS-based meningitis vaccine was developed by GSK, which was plain polysaccharide. However, polysaccharides induce only T cell-independent immunities with poor immunological memory, especially in infants and young children, and are thus not appropriate for sustained protection against infectious diseases. To address the issue, CPSs have been coupled with immunologically active carrier proteins, such as a diphtheria toxin mutant CRM197, to form conjugate vaccines that have exhibited improved efficiency and, more importantly, elicited T cell-dependent immunities. Glycoconjugate vaccines have been used for meningitis control. However, conjugate vaccines currently in clinical uses are composed of heterogeneous and easily contaminated natural CPSs that can barely meet modern quality and safety standards and demands.

To overcome these limitations, conjugate vaccines made of synthetic carbohydrate antigens, which have defined structures, uncompromised purity and reproducibility, and free of bacterial contaminants, have been explored in the our laboratory. In this regard, we used the synthetic oligosaccharide analogs of the bacterial CPS as antigens for vaccine design.

The most characteristic CSP isolated from group C N. meningitidis is α-2,9-ploysialic acid with occasional and sporadic 8-O-acetylation (FIG. 34). Reports have shown that while de-O-acetylation of this antigen could improve its immunogenicity, the provoked immune response could still recognize and kill the bacterium, thus current glycoconjugate vaccines against group C meningitis are composed of α-2,9-ploysialic acid free of O-acetylation. Accordingly, we designed and prepared a series of α-2,9-oligosialic acids without 8-O-acetylation and coupled them with a carrier protein to formulate glycoconjugate vaccines 152-155 (FIG. 34), which were evaluated in mice to analyze their structure-activity relationships. In the model study, the carrier protein used was keyhole limpet hemocyanin (KLH), as it is inexpensive and easily accessible, but the synthetic oligosaccharide can be coupled with carrier proteins such as TT, DT, CRM₁₉₇, and so on to formulate more functional vaccines. In addition, the human serum albumin (HSA) conjugates 156-159 of these α-2,9-oligosialic acids were also prepared and used as capture reagents for enzyme-linked immunosorbent assays (ELISA) of α-2,9-oligosialic acid-specific antibodies.

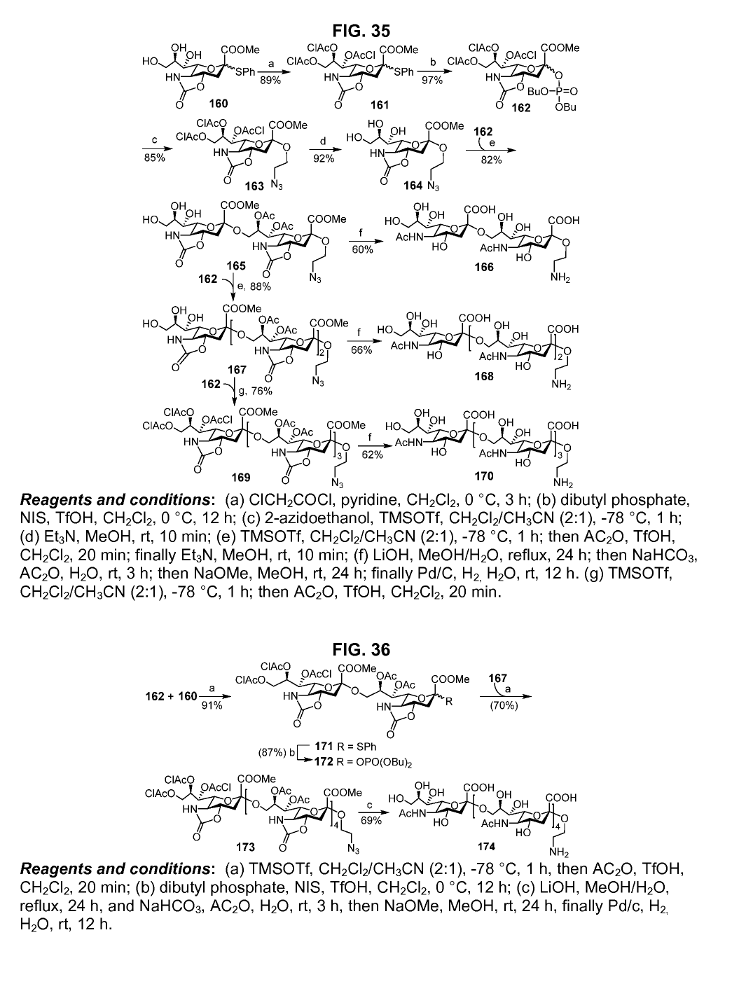

Described in FIG. 35 and FIG. 36 is the synthesis of α-2,9-oligosialic acids having a reactive 2-aminoethyl group as an appendage at the reducing end to facilitate their coupling with carrier proteins. Although there were reports in the literature about the synthesis of some α-2,9-oligosialic acids, they were prepared by different methods and were not completely deprotected or coupled with carrier proteins to form conjugates and investigated as vaccines.

Our synthesis, as shown in FIG. 35, was commenced with the preparation of 160 from sialic acid. It was then converted into the key building block 161 as an α,β-mixture in two steps and an 86% yield. Rather than spending much effort on separating the two anomers, we probed the direct use of this mixture for sialylation. Delightfully, we found that the reaction between 2-azidoethanol and 161 in a mixture of CH₂Cl₂ and CH₃CN (2/1) at −78° C. to −40° C. with TMSOTf as the promoter was α-specific to give the desired anomer 163 exclusively in an excellent 85% yield. The anomeric configuration of 163 was proved by its ¹H and ¹³C NMR data. This result suggested that both isomers of 162 could be activated and react with the glycosyl acceptor to give α-product. Next, the chloroacetyl (ClAc) groups in 163 were selectively removed with triethylamine (Et₃N) in MeOH to produce triol 164. Taking advantage of the higher reactivity of the primary hydroxyl group than secondary hydroxyl groups in 164, it was directly used for sialylation with 162 under the above condition to furnish regioselective glycosylation. The product was acetylated and then de-O-chloroacetylated as described above to produce partially protected disialic acid 165 in an 82% yield in three steps. The newly formed α-sialyl bond in 165 was confirmed by its NMR spectra. Moreover, the chemical shifts of its H-3eq signals (δ: 2.94 and 2.89 ppm) were consistent with the empirical rules about the anomeric configurations of N5,O4-carbonyl oligosialic acids described in the literature. The α,β-mixture of 162 as a sialyl donor was again very efficient and gave exclusively α-sialylation. We believed that the solvent used for the reaction might have a significant impact, as the reaction of 162 and 2-azidoethanol performed in pure dichloromethane gave a mixture (α/β 10:1). The partially protected disialic acid 165 was finally subjected to a series of reactions including deacylation with LiOH in MeOH/H₂O, peracetylation with Ac₂O, selective de-O-acetylation with NaOMe in MeOH, and then reduction of the azide group to obtain free disialic acid 166 in a 60% overall yield, which was purified by size exclusion column chromatography and characterized with 1 D, 2D NMR and HR MS.

Trisialic and tetrasialic acids were prepared from disialic acid 165 by the same strategy (FIG. 35). Glycosylation of 165 with 162 and protecting group manipulation gave 167 in an excellent overall yield (88%). Compared to the reaction of 164, the longer sugar chain in 165 did not affect the efficiency of glycosylation. Thereafter, a part of 167 was deprotected to obtain free trisialic acid 168, and the remaining 167 was sialylated with 162 and acetylated to provide 169 in a 76% overall yield. Finally, 169 was deprotected by the above protocol to furnish free tetrasialic acid 170. Compounds 168 and 170 were characterized, and both sialylation reactions were α-selective.

For pentasialic acid synthesis, we adopted a convergent [2+3] glycosylation strategy (FIG. 36), rather than direct linear elongation of the sugar chain of 169. First, 160 was sialylated with 162 under the conditions established above to obtain disialic acid 171 that was converted into sialyl phosphate 172 as a glycosyl donor. The coupling reaction between disialic acid donor 172 and trisialic acid acceptor 167 in the presence of TMSOTf was followed by O-acetylation to give 173 in a good yield (70%). Evidently, the size of sialyl donor did not significantly affect the glycosylation efficiency either. These results indicated that more complex oligosialic acids may be prepared via a convergent [n+n] or [n+m] strategy. Finally, 173 was deprotected as described above to furnish free pentasialic acid 174, which was characterized with 1D, 2D ¹H and ¹³C NMR and HR MS.

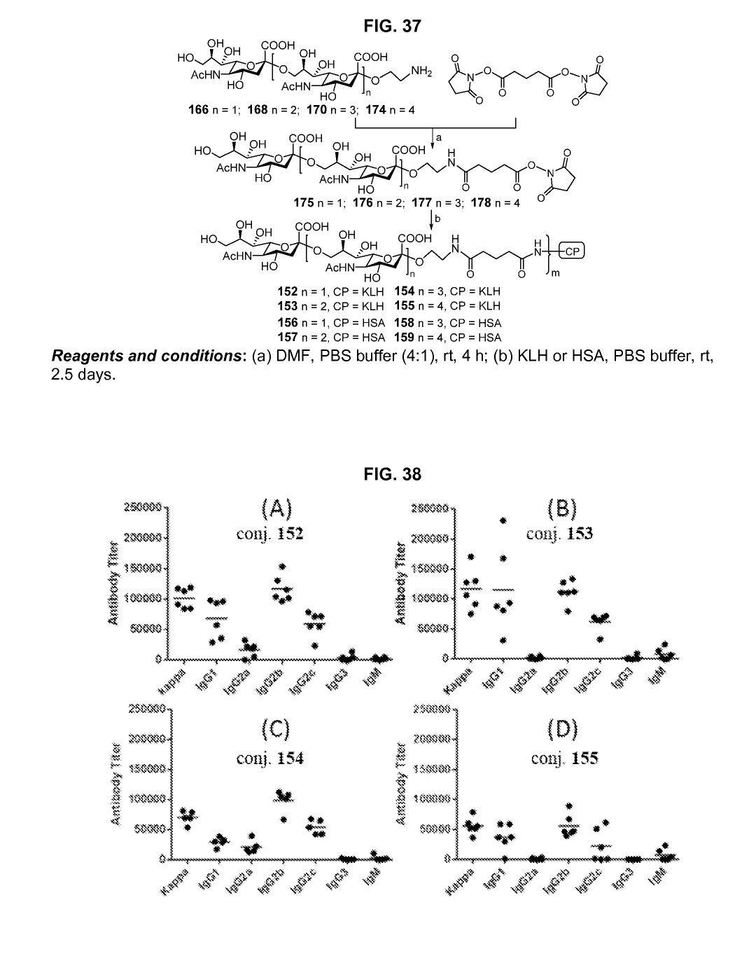

Once the oligosialic acids were available, they were conjugated with KLH and HSA via the bifunctional glutaryl linker (FIG. 37) by the same methods described in section [00106] The sialic acid contents of the resultant glycoconjugates were determined by the Svennerholm method, and the results of HSA conjugates 156-159 were also validated with MS. The sialic acid loadings of 152-159 were 7.5-11.5%, indicating that the antigen loading levels were in the desired range for glycoconjugate vaccines or for capture reagents used in ELISA.

Immunological evaluations of glycoconjugates 152-155 were carried out with 5/6-week-old female C57BL/6J mouse by the same methods and protocols described in section [00109]. FIG. 38 gave the ELISA results of day 38 antisera obtained from mice inoculated with 152-155. All of the conjugates elicited high titers of antigen-specific total antibodies, indicating that they induced strong immune responses.

The assessment of individual antibody isotypes revealed that all of the conjugates elicited mainly IgG1, IgG2b, and IgG2c antibodies (FIG. 38) and only low levels of IgM antibodies were observed. In consistent with literature report that C57BL/6 mouse does not have the IgG2a gene but expresses the IgG2c isotype instead, no significant level of IgG2a antibody was observed with the antisera. The production of IgG antibodies indicated the induction of T cell-mediated immunities and the switching of carbohydrate antigens from traditionally T cell-independent to T cell-dependent antigens through conjugation with a carrier protein. It was also reported that IgG antibody responses were associated with cellular immunity, long-term immunological memory, maturation of antibody affinity, and improved antibody-mediated cell or complement-dependent cytotoxicity, which are important and desirable for prophylactic vaccines. The subclasses of IgG antibodies are defined according to their different Fc regions and differ in their ability to activate the immune system. It was reported that the activity hierarchy for IgG antibodies was: IgG2a IgG2b>IgG1>>IgG3. The incitement of high titers of IgG1, IgG2b, and IgG2c antibodies, the latter of which is allelic to IgG2a, by 152-155 suggested their likely protective activity against N. meningitidis. Moreover, among various subclasses of IgG antibodies, IgG2b and IgG2a are believed to be the most potent ones for the activation of effector response and antiviral immunity, which further supports the protective activity of these conjugates as antibacterial vaccines.

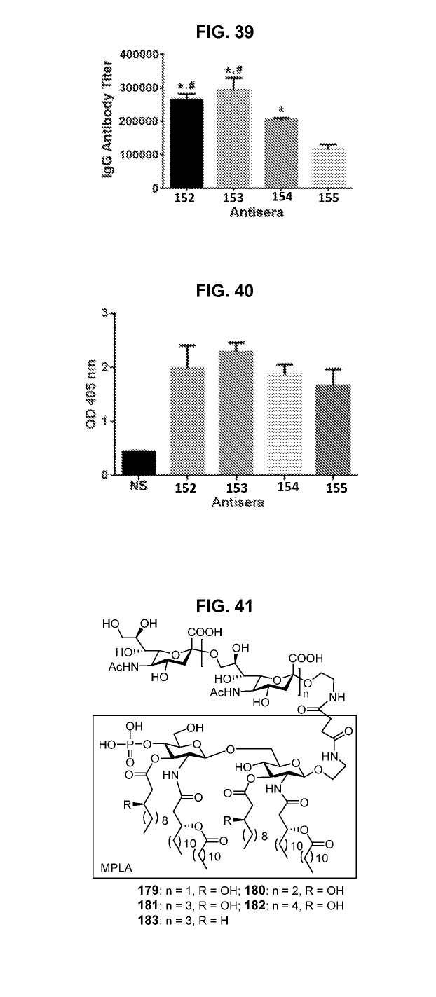

FIG. 38 also discloses that 153 elicited a higher level of IgG1 antibody than 152, but their IgG2b and IgG2c antibody levels were similar. Both elicited significantly higher IgG1, IgG2b, and IgG2c antibody titers than 153 and 155. It was further revealed that the total IgG antibody titer for 153 was slightly higher than that for 152 and significantly higher than that for 154 and 155 (FIG. 43). These results clearly suggested that the immunogenicity of the tested oligosialic acids followed the order of tri->di->tetra->penta. Consequently, trisialic acid was identified as the most promising oligosialic acid antigen for the development of group C meningitis vaccines. FIG. 39 shows the average titers of antigen-specific total IgG antibodies in the day 38 antisera of individual mice inoculated with 152-155. Error bar shows the standard error of mean for each group of mice. The difference is statistically significant (P<0.05) as compared to 4 (*) or 3 (#).

The next important question was whether the elicited antibodies or immunities could recognize and target group C N. meningitidis. This is directly related to the efficacy of the glycoconjugates as vaccines. To answer this question, we studied the binding between the antisera and group C N. meningitidis cell using normal mouse serum as the negative control. As shown in FIG. 40, all of the antisera obtained from mice inoculated with 152-155 had very strong binding to N. meningitidis cell, but no significant binding to cells not expressing α-2,9-poly/oligosialic acids, although these cells carry sialoglycans. Moreover, the antisera did not bind to silaoglycans sTn, GM3, GM2, and α-2,8-linked polysialic acid either. These results indicated that the antibodies induced by 152-155 could specifically recognize and target α-2,9-linked polysialic acid and group C N. meningitidis. The results of binding assays of group C N. meningitidis cell with 1:100 diluted normal serum (NS) or 1:100 pooled antisera derived from mice immunized with 152-155. The error bar shows the standard deviation of three parallel experiments. The difference between NS and all anti152-155 was statistically significant (P<0.05).

In summary, α-2,9-di-, tri-, tetra- and pentasialic acid derivatives were efficiently synthesized and coupled with KLH. The immunological properties of the resulting glycoconjugates 152-155 were studied in mice. It was discovered that all of the conjugates elicited robust T cell-mediated immunities desirable for prophylactic vaccines. It was also found that the order of immunogenicity of the oligosialic acids was tri->di->tetra->penta, suggesting that larger glycans are not necessarily better immunogens. To the best of our knowledge, this is the first systematic immunologic study of oligosialic acids, although several oligosialic acids were synthesized previously. It was further demonstrated that the elicited antibodies or immunities were specific to α-2,9-polysialic acid-expressing group C N. meningitidis cell. The binding of antibody to bacterial cell was very strong even with 1:100 and more diluted antisera, while usually original antisera were used for similar study in the literature. It was concluded that α-2,9-trisialic acid is a promising antigen for the development of functional vaccines for group C meningitis, and we are currently optimizing the carrier molecule for α-2,9-trisialic acid-based vaccines.

Based on our discoveries about the immunostimulatory and adjuvant property of MPLA, we envisioned that MPLA might be employed to couple with synthetic repeating unit oligosaccharides of bacterial polysaccharide antigens to generate fully synthetic, self-adjuvanting conjugate vaccines against bacteria, such as group C Neisseria meningitidis. Therefore, the above synthetic oligosaccharides of α-2,9-ploysialic acid were also coupled with MPLA, and the resultant glycoconjugates were carefully evaluated as antibacterial vaccines.

The α-2,9-di-, tri-, tetra-, and penta-sialic acids were coupled with two MPLAs, including the 4′-O-monophosphoryl form of natural lipid A of N. meningitidis (in conjugates 179-182, FIG. 41) and its analog without the hydroxyl groups on the lipid side chains (in conjugate 183). The conjugates were then evaluated in mice. The synthetic α-2,9-oligosialic acids were also coupled with keyhole limpet hemocyanin (KLH) and human serum albumin (HSA), as described above, and the resultant conjugates were used as the positive controls and capture reagents for enzyme-linked immunosorbent assay (ELISA) of α-2,9-oligosialic acid-specific antibodies, respectively.

Synthesis of Glycoconjugates 179-183.

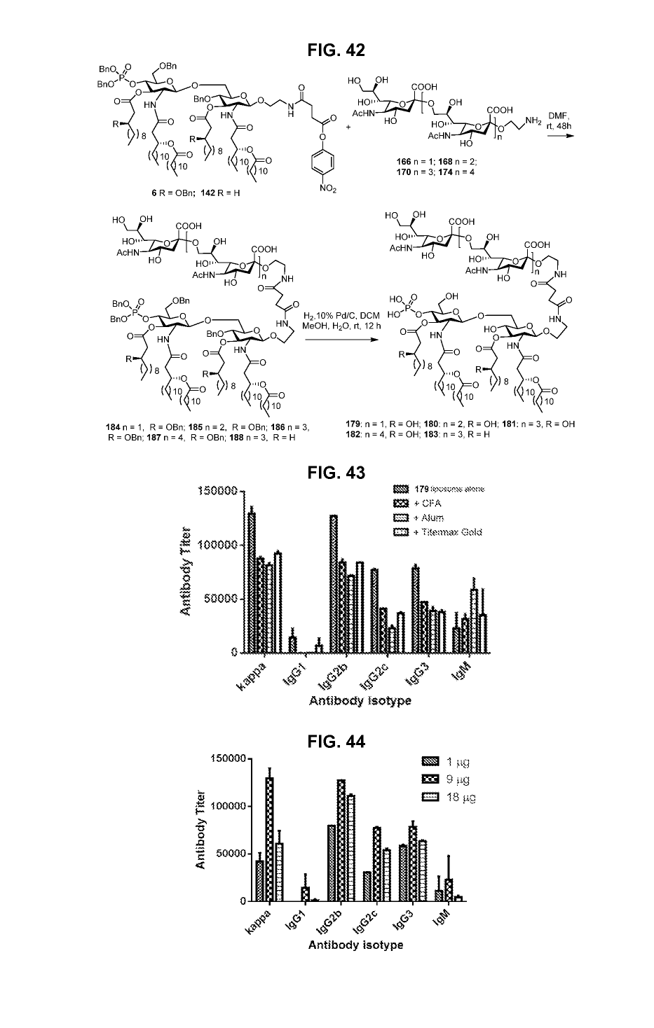

As outlined in FIG. 42, the synthesis of 179-183 started with the preparation of MPLA active esters 6 and 142 with a free carboxylic group and oligosialic acids 166-174 carrying a free amino group at the reducing end according to the procedures described above. Then, the active esters 6 and 142 were coupled with 166-174 regioselectively to produce partially protected conjugates 184-188. Finally, 184-188 were subjected to 10% Pd/C-catalyzed hydrogenolysis under an H₂ atmosphere to remove all of the benzyl ether protecting groups and afford the desired MPLA conjugates 179-183. On the other hand, the KLH and HSA conjugates of oligosialic acids were prepared by coupling 166-174 with KLH and HSA through the bifunctional glutaryl linker.

Immunologic Evaluation of Glycoconjugates 179-183.

Immunologic studies of 179-183 were carried out with female C57BL/6J mouse using homogeneous liposomal preparations of 179-183 made with DSPC and cholesterol in a 10:65:50 molar ratio, according to a reported method. The liposomal formulation by the same methods and protocols described in sections [0072] and [0073].

Influence of External Adjuvants on Immune Responses to Conjugate 179.

Currently, all clinical vaccines are used with an adjuvant. Our previous studies have revealed that MPLA conjugates as anticancer vaccines might be self-adjuvanting. To probe the influence of adjuvants on the immunologic properties of MPLA-based synthetic antibacterial vaccines, we evaluated in mice 179 alone or 179 with complete Freund's adjuvant (CFA), alum and TiterMax Gold adjuvant. Antisera were prepared from blood samples obtained 10 days after the last boost immunization and analyzed by ELISA with disialic acid-HSA conjugate as the capture reagent, and the results are depicted in FIG. 43. Evidently, 179 elicited similar immunologic responses in all four groups of mice, with the production of high titers of IgG2b and total antibodies and moderate levels of IgG2c, IgM and IgG3 antibodies, so external adjuvants had no or little impact on the immunologic responses. The production of high IgG antibody titers indicated robust T cell-mediated immunity. Thus, conjugate 179 was verified to be self-adjuvanting and elicit robust, antigen-specific T cell-mediated immunity in mice without the use of an external adjuvant.

FIG. 43 shows ELISA results of disialic acid-specific total (anti-kappa), IgG1, IgG2b, IgG2c, IgG3, and IgM antibodies elicited by MPLA conjugate 179 in the liposomal form alone or in combination with an adjuvant, including CFA, alum, and TiterMax Gold adjuvant. The error bar represents the standard error of three parallel experiments.

Influence of Vaccine Dosage on Immune Responses to Conjugate 179.

To study the dose-immunity correlation of conjugate 179, three groups of mice were immunized with the liposomal preparation of 179 containing 1, 9, and 18 μg of disialic acid per injection, respectively. The titers of total and various isotypes of antibodies were detected by ELISA. The results (FIG. 44) clearly indicated that the dosage had a small impact on the antibody titer, and mice in the 9 μg dose group had the highest titers of all tested antibody isotypes. However, high levels of IgG2b and total antibodies and moderate levels of IgG2c, IgM, and IgG3 antibodies were observed for all dose groups, and the dose did not have an obvious impact on the distribution of antibody isotypes. It seemed that a low dose of 179, such as 1 μg of disialic acid per injection, was sufficient to elicit robust T cell-mediated immunity. Although increased doses of 179, e.g., 9 μg, might further enhance the immune responses, too high doses, e.g., 18 μg, were not necessarily helpful. FIG. 44 shows ELISA results of titers of various disialic acid-specific antibodies in the day 38 pooled antisera induced by 179 in dosages of 1, 9, and 18 μg carbohydrate antigen/mouse/injection. The error bar represents the standard error of three parallel experiments.

Comparing the Immunologic Properties of MPLA and KLH Conjugates.

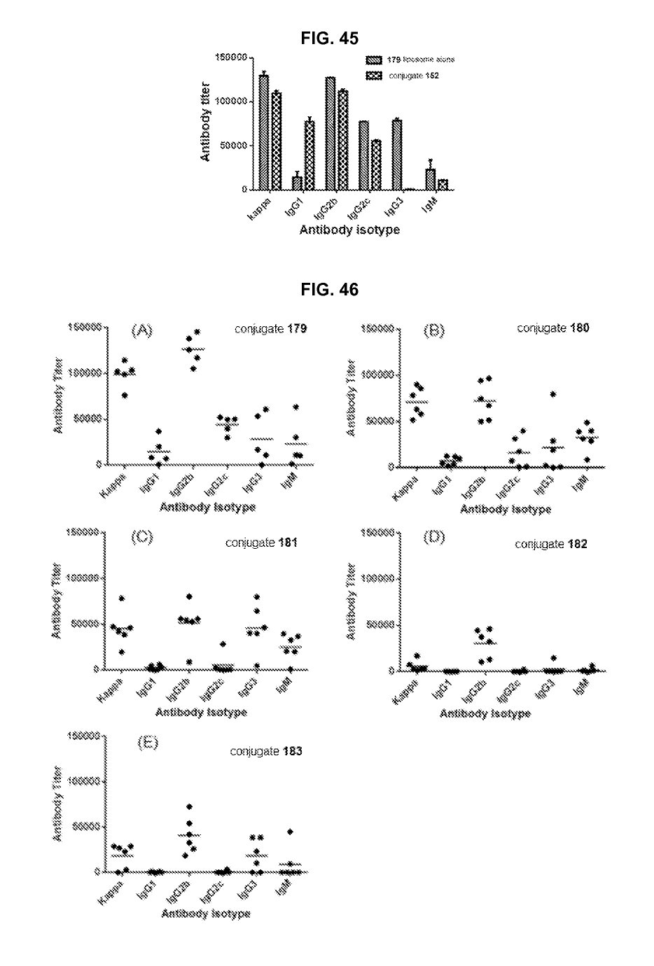

Conjugate 179 alone (9 μg of sialic acid/mouse/injection) and the corresponding KLH-disialic acid conjugate 152 (3 μg of sialic acid/mouse/injection) in emulsion with TiterMax Gold adjuvant were used to immunize mice according to the above protocols. ELISA results of the obtained antisera revealed that both conjugates provoked strong immune responses (see the total antibody titers in FIG. 45) and that the induced responses were of similar pattern, namely that both mainly elicited IgG2b and IgG2c antibodies and some IgG1 and IgG3 antibodies as well. Moreover, while the IgG1 antibody titer for 179 was slightly lower than that for the KLH conjugate, its IgG3 antibody titer was higher than that for the latter. The results were consistent with the reports that usually glycoproteins elicit IgG1 antibody responses while glycolipids often elicit carbohydrate-specific IgG3 antibody responses. Most importantly, both 179 and the corresponding KLH conjugate elicited similarly high levels of IgG2b and IgG2c antibodies that are relevant to T cell immunity. These studies further proved that 179 alone without an external adjuvant could provoke robust immune responses comparable to that elicited by the KLH conjugate used with an adjuvant, although their IgG1 and IgG3 antibody titers were slightly different. FIG. 45 illustrates ELISA results of various disialic acid-specific antibody titers of pooled mouse antisera obtained with disialic acid-MPLA conjugate 179 and disialic acid-KLH conjugate. Error bar represents the standard error of three parallel experiments.

Structure-Immunogenicity Relationship Study of the Oligosialic Acid Antigens.

Immunization of mice with conjugates 179-183 and subsequent ELISA were carried out according to the same protocol described above, while the capture reagents used for ELISA were corresponding oligosialic acid-HSA conjugates. As revealed in FIG. 46, all conjugates 179-183 elicited strong immune responses, supported by the high titers of their antigen-specific total antibodies. Moreover, IgG2b antibody was the major subclass for all five vaccine groups, which as discussed above meant memorable T cell-mediated immunity. FIG. 45A-D further indicated that the total antibody titers, as well as that of IgG2b antibodies, decreased with the size increase of oligosialic acids. Therefore, it seemed that shorter oligosialic acids were generally more immunogenic than longer analogs. Nonetheless, the IgG2b antibody titer of pentasialic acid-MPLA conjugate 182 was still very high (ca. 30,491), although its total antibody titer was relatively low. In addition, glycoconjugates 183 and 181, both composed of tetrasialic acid but containing different MPLAs, induced significantly different levels of total antibody titers (FIGS. 46C and 46E), indicating that the MPLA structure had a noticeable impact on the overall immunogenicity of the conjugates as well. However, the IgG2b antibody titers induced by 183 and 181 were only slightly different (less than 2 folds), thus it seemed that 183 could still elicit strong T cell-mediated immunity. FIG. 46 illustrates ELISA results of the day 38 antisera obtained with conjugates 179 (A), 180 (B), 181 (C), 182 (D) and 183 (E). Each dot represents the antibody titer of an individual mouse, and the average titer of each group is represented by a black bar.

Reactivity of each group of antiserum with all other oligosialic acids. Cross-reactions between antisera elicited by 179-183 and all synthetic oligosialic acids analyzed by ELISA (FIG. 47) revealed that although the antiserum induced by disialic acid-MPLA conjugate 179 had very high reactivity with disialic acid, its reactivity with other oligosialic acids was significantly lower, in decreasing of tri->tetra->pentasaccharides. These results indicated that at least a portion of the antibodies elicited by 179 was specific to disialic acid or its conjugates but did not react with larger oligosialic acids. One potential explanation was that the conformation of conjugated disialic acid might be partially affected by the carrier molecule, thereby resulting in antibodies that could only recognize the specific conformers. The antiserum obtained with trisialic acid-MPLA conjugate 180 had a similar binding trend but the difference was much less significant. In contrast, the antisera elicited by 181 and 182 had essentially the same reactivity with all of the tested oligosialic acids. In addition, we found that the antisera did not have cross-reaction with other sialic acid-containing antigens, such as GM3, sTn, and α-2,8-polysialic acid. The results suggested that the antibodies elicited by 180-182 could recognize the similar unique antigenic epitope, namely, oligosialic acids in the α-2,9-linked form. FIG. 47 shows ELISA results of the cross-reactivity between pooled antisera obtained with 179-183 and various capture reagents, including di-, tri-, tetra-, and pentasialic acid-HSA conjugates. The error bar represents the standard error of three parallel experiments.

Assessment of the Binding Between Antisera and N. meningitidis Cell.

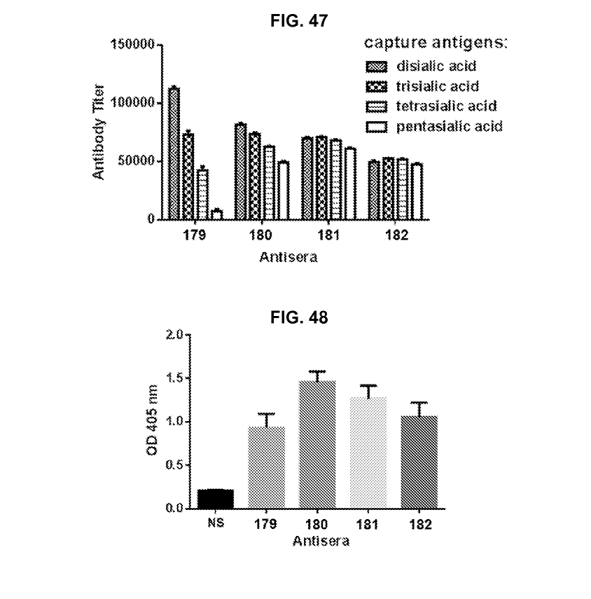

These assays were carried out on a Bio-Dot microfiltration apparatus equipped with a PVDF membrane. Pre-fixed group C N. meningitidis cell was subsequently incubated with the antisera of conjugates 179-183 and an alkaline phosphatase (AP)-conjugated antibody, and was the analyzed at 405 nm wavelength as described for regular ELISA. The results depicted in FIG. 48 proved that antibodies in these antisera could recognize and bind to bacterial cells. Interestingly, the antiserum of conjugate 179, which exhibited the highest total antibody titer, had significantly weaker binding to the bacterial cell as compared to the antisera of conjugates 180-182. Generally, the binding ability of anti-180, 181, and 182 sera to the bacterial cell was parallel to the observed antibody titers (FIG. 46). Furthermore, it was also demonstrated that the antibodies did not have significant binding to a number of cancer cell lines that express abundant sialoglycans but not α-2,9-poly/oligosialic acids. Evidently, the results indicated that a part of the antibodies elicited by conjugate 179 did not bind to α-2,9-polysialic acid on the bacterial cell, which was consistent with the cross-reactivity results discussed above (FIG. 47). The results also verified that the antibodies or immune responses induced by MPLA conjugates 179-182 could recognize and specifically target group C N. meningitidis cell. Therefore, 179-182, especially conjugates 180-182, might be effective vaccines against group C meningitis.

FIG. 48 displays results of the antiserum-N. meningitidis cell binding assay, using pooled day 38 antisera from mice immunized with conjugates 179-182, with normal sera (NS) as a control. All of the mouse sera were 1:100 diluted. The error bar shows the standard error of three parallel experiments. The difference between NS and all antisera obtained with 179-182 was statistically significant (P<0.05).

In summary: Immunological studies on α-2,9-oligosialic acid-MPLA conjugates 179-182 showed that 179-182 alone, in other words without the use of any external adjuvant, elicited high titers of both total and IgG antibodies, verifying their self-adjuvanting property. Conjugates 179-182 were injected in a liposomal form which might improve water solubility of glycolipids 179-182 and even their immunological activities. The immune response to conjugate 181, which contained a natural MPLA, was stronger than that to conjugate 183 which contained a modified MPLA but the same disialic acid antigen. Nevertheless, conjugate 183 also elicited a robust immune response. Dose-immunologic activity relationship studies revealed that the doses (1, 9, or 18 μg) of 179 used to immunize mouse also had a small impact on the intensity of induced immune response, and the highest titers of total and various isotypes of antibodies were observed with the 9 μg dose group. However, mice in all three dose groups exhibited similar and robust immune responses.

More importantly, MPLA conjugate 179 and the corresponding KLH conjugate elicited strong and similar patterns of immune responses, namely, that both had induced mainly oligosialic acid-specific IgG2b and IgG2c antibodies, as well as low levels of IgG1 and IgG3 antibodies. Similar patterns of antibody responses were also observed with other MPLA conjugates, i.e., 180-183. Robust IgG antibody responses are reported to be associated with T cell-mediated immunity, antibody affinity maturation, improved antibody-mediated cell and complement-dependent cytotoxicity, and long-term immunologic memory, which are useful and desired properties for prophylactic vaccines. The high levels of IgG antibodies, especially IgG2b and IgG2c antibodies, induced by 179-183 suggested their potential as vaccines to provide protection against group C meningitis. Moreover, among various subclasses of IgG antibodies, IgG2b and IgG2a are also the most potent antibodies for the activation of effector responses and antimicrobial immunities, which further supports the potentially protective activities of conjugates 179-183 as antibacterial vaccines.

Structure-activity relationship studies on conjugates 179-182 have demonstrated that the oligosialic acid structure had a significant impact on their immunogenicity, which decreased progressively with the increase in oligosialic acid chain length. Nonetheless, although the titers of total and IgG2b antibodies induced by conjugates 179-182 decreased with the increase of oligosialic acid chain length from di- to pentasialic acids, the pentasialic acid-MPLA conjugate 182 still elicited a sufficiently robust immune response, and its antibodies could bind to the bacterial cell effectively.

In conclusion, we have demonstrated that fully synthetic oligosialic acid-MPLA conjugates 179-183 were self-adjuvanting vaccines, which alone, without using an external adjuvant, could elicit strong T cell-mediated immunities quantitatively and qualitatively comparable to that induced by the corresponding KLH conjugate. Therefore, oligosialic acid-MPLA conjugates were identified as promising anti-group C meningitis vaccine candidates worthy further investigation.

### Methods of Treatment and Prevention of Disease Using Compounds of the Present Disclosure

The compounds of the present invention and pharmaceutical compositions comprising a compound of the present invention can be administered to a subject suffering from a cancer. Cancers can be treated prophylactically, acutely, and chronically using compounds of the present invention, depending on the nature of the disorder or condition. Typically, the host or subject in each of these methods is human, although other mammals can also benefit from the administration of a compound of the present invention.

In one aspect, the present invention relates to the co-administration of a compound of formula I-II to treat a cancer.

Formula:

M-L-A Formula I

M-L-B Formula II

In these cases, M represents an MPLA derivative, L is a linker, A is globo H, and B represents a fucose-containing TACA.
In particular embodiments, the compound of formula I-II is administered to a patient to provoke an immune response for treatment. The compound of formula I-II may be administered up to one day, one week, one to three months, one to 6 months, one year apart. In therapeutic applications, the compounds formulas I-II can be prepared and administered in a wide variety of dosage forms. The term “administering” refers to the method of contacting a compound with a subject. Thus, the compounds of the present invention can be administered by injection, that is, intravenously, intramuscularly, intracutaneously, subcutaneously, intraduodenally, parentally, or intraperitoneally. Also, the compounds described herein can be administered by inhalation, for example, intranasally. Additionally, the compounds of the present invention can be administered transdermally, topically, and via implantation. In certain embodiments, the compounds of the present invention are delivered orally. The compounds can also be delivered rectally, bucally, intravaginally, ocularly, andially, or by insufflation.

The compounds utilized in the pharmaceutical method of the invention can be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner.

Compounds of formulas I-II can be co-administered with compounds that are useful for the treatment of cancer (e.g., cytotoxic drugs such as TAXOL®, taxotere, GLEEVEC® (Imatinib Mesylate), adriamycin, daunomycin, cisplatin, etoposide, a vinca alkaloid, vinblastine, vincristine, methotrexate, or adriamycin, daunomycin, cis-platinum, etoposide, and alkaloids, such as vincristine, farnesyl transferase inhibitors, endostatin and angiostatin, VEGF inhibitors, and antimetabolites such as methotrexate. The compounds of the present invention may also be used in combination with a taxane derivative, a platinum coordination complex, a nucleoside analog, an anthracycline, a topoisomerase inhibitor, or an aromatase inhibitor). Radiation treatments can also be co-administered with a compound of the present invention for the treatment of cancers.

In another aspect, a therapeutically effective amount of an anti-TACA antibody derived from formula I-II may be administered to a cancer patient. A “therapeutically effective amount” refers to an amount, at dosages and for periods of time necessary, sufficient to inhibit, halt, or allow an improvement in the disorder or condition being treated when administered alone or in conjunction with another pharmaceutical agent or treatment in a particular subject or subject population. The term “patient” refers to a member of the class Mammalia. Examples of mammals include, without limitation, humans, primates, chimpanzees, rodents, mice, rats, rabbits, horses, dogs, cats, sheep, and cows. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

It should be appreciated that the determination of proper dosage forms, dosage amounts, and routes of administration is within the level of ordinary skill in the pharmaceutical and medical arts. A therapeutically effective amount of the antibody or compound of formula I-II may vary according to factors such as the disease state, age, sex, and weight of the individual, and the ability of the antibody to elicit a desired response in the individual. A therapeutically effective amount is also one in which any toxic or detrimental effects of an agent are outweighed by the therapeutically beneficial effects.

The antibody or compound of formula I-II may be administered once or multiple times. For example, the antibody or compound of formula I-II may be administered from three times daily to once every six months or longer. The administering may be on a schedule such as three times daily, twice daily, once daily, once every two days, once every three days, once weekly, once every two weeks, once every month, once every two months, once every three months and once every six months.

Co-administration of an antibody with an additional therapeutic agent (combination therapy) encompasses administering a pharmaceutical composition comprising the anti-TACA antibody and the additional therapeutic agent and administering two or more separate pharmaceutical compositions, one comprising the anti-TACA antibody and the other(s) comprising the additional therapeutic agent(s). Further, co-administration or combination therapy refers to antibody and/or compound of formula I-II, and additional therapeutic agents being administered at the same time as one another, as wells as instances in which an antibody and additional therapeutic agents are administered at different times. For instance, an antibody and compound of formula I-II may be administered once every three days, while the additional therapeutic agent is administered once daily. Alternatively, an antibody and compound of formula I-II may be administered prior to or subsequent to treatment of the disorder with the additional therapeutic agent. An antibody and compound of formula I-II and one or more additional therapeutic agents (the combination therapy) may be administered once, twice or at least the period of time until the condition is treated, palliated or cured.

For example, anti-TACA antibodies may be co-administered with compounds that are useful for the treatment of cancer (e.g., cytotoxic drugs such as TAXOL®, taxotere, GLEEVEC® (Imatinib Mesylate), adriamycin, daunomycin, cisplatin, etoposide, a vinca alkaloid, vinblastine, vincristine, methotrexate, or adriamycin, daunomycin, cis-platinum, etoposide, and alkaloids, such as vincristine, farnesyl transferase inhibitors, endostatin and angiostatin, VEGF inhibitors, and antimetabolites such as methotrexate. The antibody and compound of formula IV may also be used in combination with a taxane derivative, a platinum coordination complex, a nucleoside analog, an anthracycline, a topoisomerase inhibitor, or an aromatase inhibitor). Radiation treatments can also be co-administered with a compound of the present invention for the treatment of cancers.

The antibodies of the present invention can be administered by a variety of methods known in the art including, via an oral, mucosal, buccal, intranasal, inhalable, intravenous, subcutaneous, intramuscular, parenteral, or topical route. In certain embodiments, the mode of administration is parenteral (e.g., intravenous, subcutaneous, intraperitoneal, intramuscular). In certain embodiments, the antibody is administered by intravenous infusion or injection. In particular embodiment, the antibody is administered by intrarticular, intramuscular or subcutaneous injection. As will be appreciated by the skilled artisan, the route and/or mode of administration will vary depending upon the desired results.

Dosage regimens can be adjusted to provide the optimum desired response (e.g., a therapeutic response). For example, a single bolus can be administered, several divided doses can be administered over time or the dose can be proportionally reduced or increased as indicated by the exigencies of the therapeutic situation. Parenteral compositions can be formulated in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier

An exemplary, non-limiting range for a therapeutically effective amount of an antibody of the invention from 1 to 40 mg/kg. In certain embodiments, the dose is 8-20 mg/kg. In other embodiments, the dose is 10-12 mg/kg. In certain embodiments, a dose range for intrarticular injection would be a 15-30 mg/dose. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

This invention also provides for pharmaceutical compositions comprising a therapeutically effective amount of a compound of including a carbohydrate antigen as described, or a pharmaceutically acceptable salt thereof together with a pharmaceutically acceptable carrier, diluent, or excipient therefor. The phrase “pharmaceutical composition” refers to a composition suitable for administration in medical or veterinary use. The phrase “therapeutically effective amount” means an amount of a compound, or a pharmaceutically acceptable salt thereof, sufficient to inhibit, halt, or allow an improvement in the disorder or condition being treated when administered alone or in conjunction with another pharmaceutical agent or treatment in a particular subject or subject population. For example in a human or other mammal, a therapeutically effective amount can be determined experimentally in a laboratory or clinical setting, or may be the amount required by the guidelines of the United States Food and Drug Administration, or equivalent foreign agency, for the particular disease and subject being treated.

It should be appreciated that determination of proper dosage forms, dosage amounts, and routes of administration is within the level of ordinary skill in the pharmaceutical and medical arts, and is described below.

A compound of the present invention can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Preferably, a compound of the present invention will cause a decrease in symptoms or a disease indicia associated with a cancer as measured quantitatively or qualitatively.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component. In tablets, the active component is mixed with the carrier having the necessary binding properties in suitable proportions and compacted in the shape and size desired.

The powders and tablets contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from 0.01 mg to 1000 mg, preferably 0.1 mg to 100 mg, or from 1% to 95% (w/w) of a unit dose, according to the particular application and the potency of the active component. The composition can, if desired, also contain other compatible therapeutic agents.

Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

A compound of the present invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane nitrogen, and the like.

Formulations suitable for parenteral administration, such as, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a subject, in the context of the present invention should be sufficient to affect a beneficial therapeutic response in the subject over time. The term “subject” refers to a member of the class Mammalia. Examples of mammals include, without limitation, humans, primates, chimpanzees, rodents, mice, rats, rabbits, horses, livestock, dogs, cats, sheep, and cows.

The dose will be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician can evaluate factors such as the circulating plasma levels of the compound, compound toxicities, and/or the progression of the disease, etc. In general, the dose equivalent of a compound is from about 1 μg/kg to 100 mg/kg for a typical subject. Many different administration methods are known to those of skill in the art.

For administration, compounds of the present invention can be administered at a rate determined by factors that can include, but are not limited to, the LD50 of the compound, the pharmacokinetic profile of the compound, contraindicated drugs, and the side-effects of the compound at various concentrations, as applied to the mass and overall health of the subject. Administration can be accomplished via single or divided doses.

Similarly, the above methodologies can be applied to a treatment of a patient in need of an antifungal vaccine or treatment, or an antibacterial vaccine or treatment. In these cases, rather than a compound of formula I or formula II, a compound of formula III, formula IV, or formula V will be used:

M-L-D formula III

M-L-E formula IV

M-L-J formula V

In each of formula III, IV, and V, M represents a carrier protein or a monophosphorylated lipid A derivative and L is a linker. These compounds are as described throughout any portion of this disclosure. In formula III, D is a beta-glucan, particularly a synthetic beta-glucan. In formula IV, E represents a meningitis CPS-related oligosaccharide, particularly an oligosialic acid or polysialic acid, most particularly a synthetic oligosialic acid. In formula V, G represents a Hib-related oligosaccharide particularly an oligoribosylribitol phosphate, particularly a synthetic oligoribosylribitol phosphate. It is envisioned that in view of the foregoing, a completely synthetic, self-adjuvanting vaccine may be generated by synthesizing an MPLA derivative according to the teachings of U.S. Pat. No. 8,809,285 and using a linker as described herein to conjugate the MPLA derivative to a carbohydrate according to one of the above synthetic carbohydrate molecules, such as oligosialic acid or an oligoribosylribitol phosphate. Such a vaccine will be useful for treatment or prevention of bacterial infections and diseases, including those caused by group C N. meningitidis or Haemophilus influenzae B.

### Examples and Experimental Procedures

Materials, reagents, and animals. CFA, DSPC, and rabbit complements were purchased from Sigma-Aldrich. MCF-7 and SKMEL-28 cancer cells, Dulbecco's Modified Eagle's Medium (DMEM) used for cell culture, and fetal bovine serum (FBS) were purchased from American Type Culture Collection (ATCC). Penicillin-streptomycin and trypsin-EDTA were purchased from Invitrogen. Alkaline phosphatase (AP)-linked goat anti-mouse kappa, IgM, IgG1, IgG2b, IgG2c, and IgG3 antibodies and FITC-labeled goat anti-mouse kappa antibody were purchased from Southern Biotechnology. Female C57BL/6J mice of 6-8 weeks old used for immunological studies were purchased from the Jackson Laboratory. LDH Cytotoxicity Detection Kit was purchased from Takara Bio Inc.

General Experimental Methods. Chemicals and materials were obtained from commercial sources and were used as received without further purification unless otherwise noted. MS 4 Å was flame-dried under high vacuum and used immediately after cooling under a N₂ atmosphere. Analytical TLC was carried out on silica gel 60 Å F254 plates with detection by a UV detector and/or by charring with 15% (v/v) H₂SO₄ in EtOH. NMR spectra were recorded on a 400, 500, or 600 MHz machine with chemical shifts reported in ppm (δ) downfield from tetramethylsilane (TMS) that was used as an internal reference. For the sake of clearance, the NMR and other spectroscopic data of the synthetic compounds, excep for some key intermediates and products, are not presented, which are reported in the realted papers.

Methods for Synthesis of Globo H, its Conjugates and Related Studies

Compound 7. To a stirred solution of 6 (12 mg, 5 μmol) and 5 (6 mg, 8 μmol) in DMF (1.5 mL) was added N-methylmorpholine (NMM, 6 μL, 54 μmol) at 0° C. After the reaction mixture was stirred at rt 48 h, DMF was removed in vacuum. The residue was purified on a TLC plate (MeOH/CH₂Cl₂/H₂O/DMF 3:3:1:1, v/v) to get 7 as a white powder (8 mg, 55%).

Compound 1. A mixture of 7 (7.5 mg, 2.64 μmol) and 10% Pd-C (5.0 mg) in CH₂Cl₂ and MeOH (3:1, 4 mL) was stirred under an atmosphere of H₂ at rt for 12 h. Thereafter, the catalyst was removed by filtration through a Celite pad, and the Celite pad was washed with a mixture of CH₂Cl₂, MeOH and H₂O (1:1:1) and then with MeOH. The combined filtrates were concentrated in vacuum to afford glycoconjugate 1 as a white floppy solid (4.0 mg, 62%). ¹H NMR (600 MHz, CDCl₃:CD₃OD:D₂O=5:3:1): δ 5.13 (br, 1H, lipid-H-3′), 5.07-5.28 (br, 1H, lipid-H-3), 4.91 (br, 2H, 2H of lipid), 1.96 (s, 3H, NHAc); 1.81-1.56 (m, 12H, lipid), 1.53-1.11 (br, 98H, 48×CH₂, lipid), 1.05-0.85 (18H, 6 CH₃, lipid). ³¹P NMR (400 MHz, CDCl₃:CD₃OD:D₂O=5:3:1): δ −2.726; MS (ESI): calcd. for C₁₃₄H₂₄₅KN₆O₅₄P [M+K+NH₄]²⁺ m/z, 1436.3; found, 1436.9.

Compound 8: A mixture of hexasaccharide 5 (3 mg) and disuccinimidal glutarate (DSG) (15 eq) in DMF and 0.1 M PBS buffer (4:1, 0.5 ml) was stirred at rt for 6 h. The reaction mixture was concentrated under vacuum and the residue was washed with EtOAc 10 times. The resultant solid was dried under vacuum for 1 h to obtain activated oligosaccharide 8 that was directly used for conjugation with KLH and HSA.

General procedure for conjugation of 8 with HSA and KLH: A mixture of the activated oligosaccharide 8 and KLH or HSA (5 mg) in 0.4 ml of 0.1 M PBS buffer was gently stirred at rt for 2.5 days. The mixture was purified on a Biogel A 0.5 column with 0.1 M PBS buffer as the eluent. The combined fractions containing the glycoconjugate indicated by the bicinchoninic acid (BCA) assay for proteins were dialyzed in distilled water for 1 day, and lyophilized to give glycoconjugates 2 and 3 as white floppy solids.

Protocols to prepare vaccine formulations. Liposomal formulations of glycoconjugate 1 were prepared by a previously reported protocol. Briefly, after the mixture of conjugate 1 (0.5 mg, 0.17 μmol, for 30 doses), 1, 2-distearoyl-sn-glycero-3-phosphocholine (DSPC) (0.87 mg, 1.1 μmol), and cholesterol (0.33 mg, 0.85 μmol) (in a molar ratio of 10:65:50) was dissolved in a mixture of CH₂Cl₂, MeOH and H₂O (3:3:1, v/v, 2 mL), the solvents were removed under reduced pressure at 60° C. through rotary evaporation, which generated a thin lipid film on the vial wall. This film was hydrated by adding 3.0 mL of HEPES buffer (20 mM, pH 7.5) containing 150 mM of NaCl and shaking the mixture on a vortex mixer. The resultant suspension was sonicated with a sonicator for 20 min to afford the liposomal formulation used for immunizations. The average diameter of the liposomes was 1429.2±249 (SD) nm with the polydispersity index (PDI) around 0.5832. The protocol for preparing CFA emulsions of the globo H-KLH conjugate 2 was similar to that reported in the literature.56 Generally, 2 (1.13 mg) was dissolved in 1.5 mL of 1×PBS buffer and thoroughly mixed with CFA (1.5 mL) according to the manufacturer's instructions to generate the emulsion.

Mouse immunization. Each group of six female C57BL/6J mice (6-8 weeks of age) was inoculated with subcutaneous (s.c.) injection of 0.1 mL of the liposomal formulation or the CFA emulsion of a specific conjugate on day 1. Following the initial inoculation, mice were boosted 3 times on day 14, day 21, and day 28 via s.c. injection of the same conjugate formulation. Mouse blood samples were collected prior to the initial immunization on day 0 and after immunization on day 21, day 27 and day 38, and were clotted to obtain sera that were stored at −80° C. before use. The animal protocol (#A 02-10-14) for this study was approved by the Institutional Animal Care and Use Committee (IACUC) of Wayne State University, and all of the animal experiments were performed in compliance with the relevant laws and institutional guidelines.

ELISA protocol. ELISA plates were coated with a solution of the globo H-HSA conjugate 3 (2 μg/mL, 100 μL) in the coating buffer (0.1 M bicarbonate, pH 9.6) at 37° C. for 1 h and then treated with a blocking buffer, i.e., 1% BSA in PBS buffer containing 0.05% Tween-20 (PBST), followed by washing with PBST 3 times. Subsequently, a pooled or an individual mouse serum with serial half-log dilutions from 1:300 to 1:656100 in PBS was added to the coated plates (100 μL/well). The plates were incubated at 37° C. for 2 h and then washed with PBST and incubated at rt for another hour with a 1:1000 diluted solution of AP-linked goat anti-mouse kappa, IgG1, IgG2b, IgG2c, IgG3, and IgM antibody (100 μL/well), respectively. Finally, the plates were washed with PBST and developed with a p-nitrophenylphosphate (PNPP) solution in buffer (1.67 mg/mL, 100 μL) at rt for 1 h, followed by colorimetric readout using a microplate reader (ELX800, Bio-Tek instruments Inc.) at 405 nm wavelength. For titer analysis, the OD values were plotted against the serum dilution numbers to obtain a best-fit logarithm line. The equation of this line was used to calculate the dilution number at which an OD value of 0.1 was achieved, and this dilution number is defined as the antibody titer.

Protocols for cytokine assay. Mouse cytokine antibody array-membrane (ab133993) was purchased from abcam for detection of mouse cytokines in the day 38 antiserum according to the manufacturer's instruction, using the day 0 normal mouse serum as negative control. First, each membrane was blocked with the blocking buffer provided within the kit at room temperature for 30 min. Then, the membrane was incubated with the mouse serum (1:5 diluted in blocking buffer, 100 μl) at 4° C. overnight. After washing, the membrane was incubated with Biotin-conjugated anti-cytokine antibodies at room temperature for 2 h. The membrane was washed again and then incubated with HRP-conjugated Streptavidin. The membrane was finally detected by using an X-ray film after addition of the chemiluminescence buffer. The summed signal intensity of positive control was set as 1, and that of the negative control as 0. The relative intensity of each cytokine in the serum was calculated according to the equation shown below:

Relative intensity of a cytokine=(signal density of the cytokine spot−signal density of negative control)/(signal density of positive control−signal density of negative control)

Protocols for FACS assay. Globo H-expressing MCF-7 and globo H-negative SKMEL-28 cell lines were used in the experiments. MCF-7 cell was incubated in ATCC-formulated Eagle's Minimum Essential Medium (EMEM) containing 10% FBS and 1% antibiotics, and SKMEL-28 cell was incubated in ATCC-formulated DMEM containing 10% FBS and 1% antibiotics. Both were harvested after treatment with trypsin-EDTA solution. Cells (about 1.0×106) were washed twice with FACS buffer (PBS containing 5% FBS) and incubated with 50 μL of normal mouse serum (1:10 dilution) or a day 38 pooled antiserum (1:10 dilution) at 4° C. for 30 min. Thereafter, the cells were washed again with FACS buffer and incubated with FITC-linked goat anti-mouse kappa antibody (2 μL in 50 μL FACS buffer) at 4° C. for 30 min. Finally, cells were washed and suspended in 0.8 mL of FACS buffer for FACS analysis on a Becton Dickinson LSR II Analyzer at the Microscopy, Imaging and Cytometry Resources Core, Wayne State University.

Protocols for CDC assay. CDCs were determined using the LDH Cytotoxicity Detection Kit according to manufacture's instructions. MCF-7 (1.0×104 cells/well) and SKMEL-28 (1.5×104 cells/well) cells were seeded in 96-well plates and then incubated at 37° C. overnight. After washing, 100 μL of a normal mouse serum (1:50 dilution) or a day 38 antiserum (1:50 dilution in medium) was added to each well, and the plates were incubated at 37° C. for 2 h. The cells were washed and then incubated with 100 μL of rabbit complement serum (1:10 dilution) at 37° C. for 1 h. For the low control (background of LDH release), no mouse antiserum was added, while for the high control (maximum LDH release) the rabbit complement serum was replaced with 100 μL of 1% tritone-100. After incubation, 20 μL of supernatant was carefully transferred from each well into new 96-well plates containing 80 μL of PBS in each well. Then, 100 μL of the LDH Cytotoxicity Detection reagent was added to each well. The mixture was incubated at rt for 1 h. The optical absorption (A) of each well was read at 490 nm wavelength with a plate reader. The percentage of cell lysis is calculated according to the following the equation:

Cell lysis %=(experimental A−low control A)/(high control A−low control A)×100% where “experimental A” is the optical absorption at 490 nm of analyzed cells treated with a serum, “low control A” is the optical absorption of cells without serum treatment, and “high control A” is the absorption of cells completely lyzed with a 1% triton.

Compound 11. After the mixture of 10 (4.0 g, 12.8 mmol) and Bu₂SnO (3.83 g, 15.4 mmol) in anhydrous toluene (50 mL) was refluxed in a flask equipped with a Dean-Stark device to remove water for 6 h, the solvent was evaporated under reduced pressure. The residue was mixed with CsF (5.84 g, 38.46 mmol) and BnBr (2.28 mL, 19.2 mmol) in DMF (20 mL) and stirred at rt for 12 h. After the reaction was complete as indicated by TLC, DMF was removed under reduced pressure. The residue was dissolved in CH₂Cl₂ and washed with 1M aq. NaF solution. The organic phase was dried over Na₂SO₄ and condensed, and the residue was purified by column chromatography (acetone/hexane 1:9, v/v) to produce 11 (4.28 g, 83%) as colorless syrup.

Compound 12. After a solution of 11 (4.1 g, 10.1 mmol), Et₃N (2.8 mL, 20.4 mmol), BzCl (1.42 mL, 12.2 mmol) and a few drop of DMAP in anhydrous CH₂Cl₂ (30 mL) was stirred at rt overnight, the reaction mixture was washed with saturated aq. NaHCO₃ solution (3×10 mL) followed by drying over Na₂SO₄. The desired product 12 was obtained as colorless syrup (4.3 g, 84%) after flash column chromatography (acetone/hexane 1:10, v/v).

Compound 22. After a mixture of 12 (2.0 g, 3.95 mmol), TTBP (2.94 g, 11.8 mmol), NIS (1.77 g, 7.9 mmol) and AgOTf (2.03 g, 7.9 mmol) was stirred in wet CH₂Cl₂ (15 mL) at 0° C. for 2 h, the reaction mixture was allowed to warm up to rt and stirred for another 4 h. The reaction mixture was quenched with saturated aq. Na2S₂O₃ solution (10 mL) at 0° C., and the mixture was diluted with CH₂Cl₂ and washed with brine. The organic layer was dried over anhydrous Na₂SO₄ and concentrated in vacuum. The residue was purified by flash column chromatography (acetone/hexane 1:4, v/v) to afford the hemiacetal as a white solid (1.38 g, 76%, an anomeric mixture with a as the major product), which was directly applied to the next reaction. DBU (4 drop) was added to a solution of the above product (1.3 g, 2.8 mmol) and trichloroacetonitrile (1.1 mL, 14.05 mmol) in anhydrous CH₂Cl₂ (15 mL), and the solution was stirred under an Ar atmosphere at 0° C. for 1 h. The reaction mixture was concentrated in vacuum, and the product was purified with a Et3N neutralized silica gel column to get 22 (1.42 g, 81%) as a white solid.

Compound 13. It was prepared according to the same procedure used to prepare 11 except for replacing BnCl with PMBCl for the alkylation reaction. Starting from 4.0 g of 10 (12.8 mmol) and 2.6 mL of PMBCl (19.2 mmol), 4.58 g of 13 (83%) was obtained as colorless syrup.

Compound 14. To a solution of 13 (4.5 g, 10.4 mmol) dissolved in anhydrous DMF was added NaH (275 mg, 11.45 mmol) at 0° C. After 45 min of stirring, BnBr (1.85 mL, 15.62 mmol) was added to the reaction mixture at 0° C., and the reaction mixture was stirred for 6 h. When TLC showed that the reaction was completed, the reaction was quenched with H₂O at 0° C., and the mixture was diluted with EtOAc. The aq.s layer was extracted with EtOAc (5×20 mL), and the organic phases were combined and dried over Na₂SO₄. The solvent was removed under reduced pressure, and the residue was purified by flash column chromatography (acetone/hexane 1:11, v/v) to obtain 14 (4.38 g, 81%) as colorless syrup.

Compound 12. Starting from 2.15 g of 10 (9.58 mmol), 1.77 g of 15 (81%) was obtained as a white solid.

Compound 17. The solution of 16 (4.15 g, 10.1 mmol), benzaldehyde dimethyl acetal (1.82 mL, 12.10 mmol) and CSA (585 mg, 2.5 mmol) dissolved in anhydrous acetonitrile (50 mL) was stirred at rt with occasional vacuum application until TLC showed that the reaction was complete. The reaction was quenched with Et3N (0.7 mL, 5.04 mmol), and the mixture was diluted with CH₂Cl₂ (30 mL) and washed with brine. The organic phase was dried over anhydrous Na₂SO₄ and concentrated in vacuum. The residue was purified by flash column chromatography (MeOH/CH₂Cl₂, 1:9, v/v) to give 17 as a white floppy solid (3.74 g, 74.2%).

Compound 18. To the solution of 17 (3.7 g, 7.41 mmol) dissolved in anhydrous DMF (30 mL) was added NaH (1.07 g, 44.44 mmol) at 0° C. The mixture was stirred at 0° C. for 45 min, and then BnBr (6.16 mL, 51.85 mmol) was added. After stirring for another 12 h when TLC showed that the reaction was completed, it was quenched with H₂O at 0° C., and the mixture was diluted with EtOAc. The aqueous layer was extracted with EtOAc (5×25 mL), and the organic phases were combined and dried over Na2_{S}O₄. The desired product 18 (6.24 g, 89%) was obtained upon flash column chromatography (acetone/hexane 1:10, v/v) of the condensed product.

Compound 19. After the mixture of 18 (2.0 g, 2.1 mmol), NaBH₃CN (1.24 g, 21.05 mmol) and 4 Å MS (6 g) in dry THF (30 mL) was stirred at rt for 2 h, it was cooled to 0° C., and HCl (1 M in dry ether) was added dropwise until pH reached 2. The reaction mixture was stirred at 0° C. for 4 h and at rt for 8 h. When TLC showed that the reaction was completed, Et₃N (1.5 mL) was added to terminate reaction. Molecular sieves were filtered off through a Celite pat and washed with CH₂Cl₂. The filtrate and washings were combined and washed with saturated aq. NaHCO₃ solution and brine, dried over Na₂SO₄ and condensed in vacuum. The residue was purified by flash column chromatography (acetone/hexane 1:11, v/v) to give 19 as a white floppy solid (1.42 g, 70.9%).

Compound 23. The mixture of 22 (1.42 g, 2.26 mmol), 20 (950 mg, 1.89 mmol), and 4 Å MS (3.0 g) in CH₂Cl₂ (20 mL) was stirred at rt under an Ar atmosphere for 1 h. After being cooled to −78° C., TMSOTf (3.42 μL, 0.019 mmol) was added, and the reaction was stirred at −65° C. for 2 h. When TLC showed that the reaction was completed, saturated aq. NaHCO₃ was added to quench the reaction, and it was then diluted with CH₂Cl₂. Molecular sieves were removed by passing through a Celite pad. After extraction of the aq. layer with CH₂Cl₂ (3×10 mL), the combined organic phase was dried over Na₂SO₄ and concentrated in vacuum, and the residue was purified by silica gel flash column chromatography (acetone/hexane 1:11, v/v) to give 23 (1.34 g, 75%) as colorless syrup.

Compound 24. After the mixture of 19 (1.4 g, 1.47 mmol) and 4 Å MS (4 g) in CH₂Cl₂ (20 mL) was stirred at rt under an Ar atmosphere for 1 h, it was then cooled to −78° C. Then, TMSOTf (2.66 μL, 0.015 mmol) was added, which was followed by dropwise addition of 15 (1.74 g, 2.79 mmol) dissolved in anhydrous CH₂Cl₂. The reaction was stirred at the same temperature for 2 h. When TLC showed the reaction was completed, saturated aq. NaHCO₃ solution was added to quench the reaction, and then CH₂Cl₂ was added for dilution. Molecular sieves were removed by passing through a Celite pad. After extraction of the aqueous layer with CH₂Cl₂ (3×10), the combined organic phase was dried over Na₂SO₄ and concentrated in vacuum, and the product was purified by silica gel column chromatography (acetone/hexane 1:11, v/v) to afford 24 (1.2 g, 58%, colorless syrup as the only trisaccharide.

Compound 25. After the mixture of 24 (1.0 g, 0.708 mmol) and DDQ (322 mg, 1.42 mmol) in CH₂Cl₂ and H₂O (9:1, 12 mL) was stirred at 0° C. for 1 h, it was poured into saturated aq. NaHCO₃ solution (50 mL). The mixture was extracted with CH₂Cl₂ (3×10 mL), and the organic payer was washed with saturated aq. NaHCO₃ solution (3×10 mL) and water (50 mL), dried over Na₂SO₄, and then concentrated in vacuum. The crude product was purified with silica gel column chromatography (acetone/hexane 1:11, v/v) to give 25 (790 mg, 86.3%) as colorless syrup.

Compound 26. After the mixture of 23 (917 mg, 0.967 mmol), 25 (500 mg, 0.387 mmol) and 4 Å MS (3 g) in CH₂Cl₂ (20 mL) was stirred at rt under an Ar atmosphere for 1 h, it was cooled to −50° C., and then NIS (261 mg, 1.16 mmol) and AgOTf (298 mg, 1.16 mmol) were added. The mixture was allowed to warm up to −30° C. and was stirred at this temperature for 2 h. When TLC showed the completion of reaction, saturated aq. NaHCO₃ solution was added to quench the reaction, and CH₂Cl₂ was then added for dilution. Molecular sieves were removed by passing the mixture through a Celite pad. After extraction with CH₂Cl₂ (3×10), the organic phases were combined and washed with saturated aq. Na2S2O3 solution, dried over Na₂SO₄, and then concentrated in vacuum. The crude product was purified by silica gel column chromatography (acetone/hexane 1:9, v/v) to afford 26 (530 mg, 64.6%) as colorless syrup.

Compound 27. After the solution of 26 (0.50 g, 0.63 mmol) and NH₂NH₂.H₂O (3.5 mL) in EtOH (10 mL) was refluxed for ca. 6 h, MALDI TOF MS [positive mode: calcd. for C₁₀₉H₁₁₆N₄O₂₅ [M+Na]⁺ m/z, 1905.1; found, 1905.0] showed that both the Phth group and the Bz group were completely removed. The mixture was concentrated in vacuum, and the residue was dissolved in anhydrous acetic anhydride (5 ml) and pyridine (5 mL). The solution was stirred at rt for 5 h, and at this point, MALDI TOF MS [positive mode: calcd. for C₁₁₃H₁₂₀N₄O₂₇ [M+Na]⁺ m/z, 1989.1; found, 1988.7] showed the complete acetylation of the hydroxyl and amino group. The solution was concentrated in vacuum, co-evaporated twice with anhydrous toluene (5 mL), and then dried under high vacuum for 1 h. The solid residue (1.35 g, 3.37 mmol) was dissolved in MeOH (5 mL), to which was added the CH₃ONa/CH₃OH solution (0.4 M) until pH reached 9.5. Thereafter, the reaction mixture was heated to 70° C. for another 6 h, and MALDI TOF MS [positive mode: calcd. for C₁₁₁H₁₁₉N₄O₂₆ [M+Na]⁺ m/z, 1947.1; found, 1947.3] showed complete 0-deacetylation. The reaction mixture was neutralized to pH 6-7 using Amberlyst (H+) resin and then concentrated in vacuum. The crude product was purified by flash column chromatography (acetone/hexane, 1:7, v/v) to give 27 as a white solid (240 mg, 54%).

Compound 29. After the mixture of 28 (154 mg, 0.293 mmol), 27 (225 mg, 0.117 mmol) and 4 Å MS (3 g) in CH2Cl2 (20 mL) was stirred at rt under an Ar atmosphere for 1 h, it was cooled to −50° C., and then NIS (79 mg, 0.531 mmol) and TfOH (1.04 μL, 0.012 mmol) were added. The mixture was allowed to warm up to −30° C. and was stirred at this temperature for 2 h. When TLC showed the completion of reaction, saturated aq. NaHCO₃ solution was added to quench the reaction, and CH₂Cl₂ was then added for dilution. Molecular sieves were removed by passing the mixture through a Celite pad. After extraction with CH₂Cl₂ (3×10), the organic phases were combined and washed with saturated aq. Na₂S₂O₃ solution, dried over Na₂SO₄, and then concentrated in vacuum. The crude product was purified by silica gel column chromatography (acetone/hexane 1:6, v/v) to give 29 (192 mg, 70%) as colorless syrup.

Compound 5. The solution of 29 (80 mg) dissolved in AcOH and H₂O (5:1, 5 mL) was heated at 60° C. for 12 h, at which point MALDI TOF MS [positive mode: calcd. for C₁₃₈H₁₄₆N₄O₃₀ [M+Na]⁺ m/z, 2099.3; found, 2100.4] confirmed the removal of all benzylidene groups. The solvent was removed in vacuum and the residue was co-evaporated with toluene 5 times to afford a solid product, which was briefly purified by passing through a short silica gel column with n-hexane and ethyl acetate (2:1 to 1:2) as the eluent. The product (30.0 mg, 14 μmol) was mixed with 10% Pd-C (20.0 mg) in MeOH and H₂O (4:1, 10 ml), and the mixture was shaken under a H₂ atmosphere at 50 psi for 48 h. The catalyst was removed by filtration through a Celite pad and the pad was washed with a mixture of MeOH and H₂O (1:1). The combined filtrate was concentrated under vacuum and the residue was dissolved in 2 ml of H₂O and lyophilized to provide the crude product, which was purified twice with a sephadex G-25 gel filtration column using water as the eluent followed by lyophilization to afford 5 (16.2 mg, 50%) as a white solid. [α]_{D} 25=+9.8° (c 0.4, H₂O). ¹H NMR (600 MHz, D₂O): δ 5.04 (d, J=3.7 Hz, 1H, H-1′″″), 4.70 (d, J=2.9 Hz, 1H, H-1″), 4.43 (d, J=7.3 Hz, 1H, H-1″″), 4.38-4.34 (m, 2H, H-1″′, H-1), 4.33 (d, J=7.3 Hz, 1H, H-1′), 4.23-4.18 (m, 1H), 4.07-4.02 (m, 2H), 3.97-3.90 (m, 2H), 3.86-3.69 (m, 7H), 3.68-3.63 (m, 3H), 3.62-3.38 (m, 19H), 3.19 (d, J=8.1 Hz, 1H), 3.10-3.06 (m, 2H), 1.86 (s, 3H, —NHAc), 1.03 (d, J=6.6 Hz, 3H, ¹³C NMR (125 MHz, D₂O): δ 174.2, 103.9, 103.2, 102.0, 101.8, 100.4, 99.2, 78.6, 78.2, 77.1, 76.3, 76.0, 75.4, 75.0, 74.7, 74.5, 74.2, 73.5, 72.7, 72.0, 71.8, 70.8, 70.1, 69.4, 69.1, 69.0, 68.4, 68.0, 67.7, 66.7, 65.7, 60.9, 60.8, 60.3, 58.9, 51.6, 39.3, 22.2, 15.2; MALDI TOF MS (positive mode): calcd. for C₄₀H₇₀N₂NaO₃₀ [M+Na]⁺ m/z, 1081.98; found, 1081.991; and HRMS (ESI TOF): calcd. for C₄₀H₇₁N₂O₃₀ [M+H]⁺ m/z, 1059.4092; found, 1059.4089.

### Experimental Section for Linear β-Glucan Oligosaccharide Synthesis and Immunological Studies

Compound 39. A mixture of diol 38 (9.0 g, 24.03 mmol) and Bu₂SnO (7.18 g, 28.84 mmol) in toluene (400 mL) was refluxed in a flask equipped with a Dean-Stark device for 6 h. After the mixture was cooled to room temperature, the residual solvent was removed under vacuum. CsF (7.99 g, 52.87 mmol), 2-bromomethylnaphthalene (10.10 g, 45.66 mmol) and DMF (60 mL) were added, and the resulting solution was stirred at 70° C. for 12 h when TLC showed completion of reaction. After DMF was removed under vacuum, the residue was dissolved in CH₂Cl₂ and washed with 1 M NaF aqueous solution. The organic phase was dried over Na₂SO₄ and purified by flash column chromatography (toluene/EtOAc 10:1) to offer give 39 (8.9 g, 72%) as a white solid.

Compound 40. A solution of 39 (8.6 g, 16.71 mmol), triethyl amine (5.8 mL, 41.77 mmol), benzoyl chloride (2.33 mL, 20.05 mmol) and a catalytic amount of N, N-dimethylaminopyridine in anhydrous CH₂Cl₂ (150 mL) was stirred at room temperature overnight. The reaction mixture was washed with saturated aqueous NaHCO₃ solution (3×150 mL), and the organic layer was dried over Na₂SO₄. The desired product 40 was obtained (9.9 g, 96%) after purification by flash column chromatography (hexanes/EtOAc/CH₂Cl₂ 6:1:1) as a white solid.

General procedure for deprotection of 2-naphthylmethyl ethers. To the stirred solution of a 2-naphthylmethyl ether compound (1 mmol) in CH₂Cl₂ (18 mL) and water (1 mL) was added DDQ (2 mmol) at room temperature. After the reaction was stirred for 8 h, saturated aqueous NaHCO₃ solution was added, and the product was extracted with CH₂Cl₂. The combined organic layers were washed three times with saturated aqueous NaHCO₃ solution and dried over Na₂SO₄. After removal of the solvent in vacuum, the product was purified by silica gel chromatography.

Compound 41. It (5.6 g, 92%) was prepared from 40 (7.88 g, 12.73 mmol) and DDQ (5.78 g, 25.47 mmol) according to the general procedure for deprotection of naphthylmethyl ethers and was purified by flash column chromatography (toluene/EtOAc 15:1 to 10:1).

General procedure for pre-activation-based glycosylation reactions. After the mixture of a glycosyl donor (1 mmol) and 4 Å MS (1.5 g) in CH₂Cl₂ (20 mL) was stirred at room temperature for 1 h, it was cooled to −78° C., and AgOTf (3 mol in 6 mL dry acetonitrile) was added, followed by addition of p-toluene sulfenyl chloride (p-TolSCl) (1 mmol) via a micro-syringe 10 min later. The mixture was stirred at −78° C. for an additional 15 min, when TLC showed that the donor was completely consumed. A solution of the acceptor (1 mmol) and 2,4,6-tri-tert-butyl pyrimidine (TTBP) (1 mmol) in CH₂Cl₂ (5 mL) was added. The resulting mixture was stirred for 20 min and warmed up to room temperature, followed by filtration to remove MS. The filtrate was washed with saturated aqueous NaHCO₃ solution and brine, dried over Na2SO4, and concentrated under vacuum. The resultant crude product was purified by silica gel flash column chromatography to get the desired compound.

Compound 42. It (6.15 g, 90%) was prepared from glycosyl donor 40 (4.37 g, 7.05 mmol) and acceptor 41 (3.375 g, 7.05 mmol) according to the general procedure for pre-activation-based glycosylation and was purified by flash column chromatography (toluene/EtOAc 15:1).

Compound 43. It (1.63 g, 95%) was prepared from 42 (2.0 g, 2.05 mmol) and DDQ (0.93 g, 4.11 mmol) according to the general procedure for naphthylmethyl ether deprotection and purified by flash column chromatography (toluene/EtOAc 7:1).

Compound 44. It (3.3 g, 86%) was prepared from glycosyl donor 42 (2.23 g, 2.29 mmol) and acceptor 43 (1.91 g, 2.29 mmol) according to the general procedure for pre-activation-based glycosylation and was purified by flash column chromatography (toluene/EtOAc 12:1).

Compound 45. The reaction between 42 (3.5 g, 3.60 mmol) and 2-azidoethanol (0.5 g, 5.65 mmol) was carried out according to the general procedure for pre-activation-based glycosylation, and the crude product was directly subjected to deprotection by the general procedure to remove the naphthylmethyl group to afford 45 (2.5 g, 91%), which was purified by flash column chromatography (toluene/EtOAc 5:1).

Compound 46. It (1.53 g, 90%) was prepared from glycosyl donor 42 (1.10 g, 1.13 mmol) and acceptor 45 (0.905 g, 1.13 mmol) according to the general procedure for pre-activation-based glycosylation and was purified by flash column chromatography (toluene/EtOAc 4:1).

Compound 47. It (0.606 g, 87%) was prepared from glycosyl donor 42 (0.306 g, 0.315 mmol) and acceptor 46 (0.475 g, 0.315 mmol) by the same synthetic procedure for 46 and was purified by flash column chromatography (toluene/EtOAc 10:1 to 6:1).

Compound 48. It (0.74 g, 81%) was prepared from glycosyl donor 44 (0.53 g, 0.315 mmol) and acceptor 46 (0.47 g, 0.315 mmol) by the same synthetic procedure for 46 and purified by flash column chromatography (toluene/EtOAc 8:1 to 4:1).

Compound 51. It (0.156 g, 80%) was prepared from glycosyl donor 42 (108 mg, 0.11 mmol) and acceptor 48 (158 mg, 0.05 mmol) by the same synthetic procedure for 46 and was purified by flash column chromatography (toluene/EtOAc 10:1 to 4:1).

Compound 53. It (0.380 g, 85%) was prepared from glycosyl donor 44 (218 mg, 0.13 mmol) and acceptor 48 (300 mg, 0.10 mmol) by the same synthetic procedure for 46 and was purified by flash column chromatography (toluene/EtOAc 12:1 to 3:1).

General procedure for global deprotection of 47, 48, 51, 53: To a solution of 47, 48, 51 or 53 (23 μmol) in CH₂Cl₂ (12 mL) was added acetic acid (8 drops) and zinc powder (80 mg). The mixture was vigorously stirred at room temperature for 24 h and then filtered through a pad of Celite plug. The filtrate was condensed in vacuum, and the resulting residue was dissolved in AcOH and H₂O (5:1, 60 mL) and heated at 60° C. for 24 h. The solvents were removed in vacuum and the residue was co-evaporated with toluene 5 times. After the product was dissolved in tBuOH and H₂O (4:1, 60 mL), NaOH (120 mg) in H₂O (6.0 mL) was added in portions. The mixture was stirred at 40° C. for 24 h, neutralized with 0.25 N HCl at 0° C., and lyophilized. The crude product was purified on a sephadex G-25 gel filtration column using water as the eluent, and the product fractions were lyophilized to afford the desired free oligosaccharides.

Compound 49. It (18.7 mg) was prepared from 47 (50.0 mg, 23 μmol) by the above general procedure in an 80% yield. ¹H-NMR (600 MHz, D₂O) δ 4.59 (m, 5H), 4.39 (d, J=8.1 Hz, 1H), 3.97 (dd, J=10.9, 5.1 Hz, 1H, ½OCH₂CH₂), 3.85-3.74 (m, 6H), 3.68-3.55 (m, 10H), 3.47-3.30 (m, 13H), 3.29-3.16 (m, 4H), 3.15-3.09 (m, 2H, CH2N3). ¹³C NMR (150 MHz, D₂O) δ 102.7, 102.5, 101.8, 84.2, 84.0, 76.0, 75.6, 75.5, 73.4, 73.2, 72.7, 69.5, 68.0, 65.8, 60.6, 60.5, 60.03, 60.02, 39.4. HRMS (ESI-TOF,) m/z: calcd. for C₅₀H₈₈NO₄₁ [M+H]⁺, 1304.3775; found, 1034.3727.

Compound 50. It (10.2 mg) was prepared from 48 (25 mg, 8.5 μmol) by the above general produre in an 88% yield. ¹H-NMR (600 MHz, D²O) δ 4.57 (m, 7H), 4.35 (d, J=8.0 Hz, 1H), 3.93 (m, 1H), 3.78-3.71 (m, 8H), 3.62-3.50 (m, 16H), 3.40-3.26 (m, 22H), 3.19 (m, 3H), 3.08 (t, J=4.9 Hz, 2H). ¹³C NMR (150 MHz, D²O) δ 102.7, 102.4, 101.8, 84.1, 84.1, 84.0, 75.9, 75.5, 75.5, 73.4, 73.2, 73.1, 72.7, 69.5, 68.0, 67.9, 65.8, 60.6, 60.4, 39.3. HRMS (ESI-TOF) m/z: calcd. for C₅₀H₈₈NO₄₁ [M+H]⁺, 1358.4832; found, 1358.4775.

Compound 52. It (11.0 mg) was prepared from 51 (27.8 mg, 7.6 μmol) by the above general procedure in an 85% yield. ¹H-NMR (600 MHz, D₂O) δ 4.66-4.59 (m, 9H), 4.39 (d, J=8.24 Hz, 1H), 4.00-3.96 (m, 1H), 3.82-3.74 (m, 12H), 3.65-3.55 (m, 19H), 3.42-3.31 (m, 26H), 3.27-3.19 (m, 4H), 3.13-3.11 (m, 2H). ¹³C NMR (150 MHz, D₂O) δ 102.7, 102.5, 101.8, 84.1, 84.0, 75.9, 75.6, 73.4, 73.2, 72.7, 71.6, 69.5, 69.4, 68.0, 65.78, 60.6, 60.3, 60.0, 39.4. HRMS (ESI-TOF) m/z: calcd. for C₆₂H₁₀₈NO₅₁ [M+H]⁺, 1682.5888; found, 1682.5787.

Compound 54. It (10.4 mg) was prepared from 53 (25.6 mg, 5.9 μmol) by the above general procedure in an 88% yield. ¹H-NMR (600 MHz, D₂O) δ 4.65-4.59 (m, 11H), 4.39 (d, J=7.83 Hz, 1H), 3.98-3.93 (m, 1H), 3.80-3.74 (m, 13H), 3.65-3.54 (m, 24H), 3.42-3.31 (m, 32H), 3.27-3.18 (m, 4H), 3.08-3.06 (m, 2H). ¹³C NMR (151 MHz, D₂O) δ 102.7, 102.4, 101.8, 84.1, 84.1, 83.9, 75.9, 75.5, 73.4, 73.2, 72.7, 71.6, 69.6, 69.5, 69.3, 68.0, 65.8, 64.8, 62.4, 60.6, 60.4, 60.3, 60.0, 39.3. HRMS (ESI-TOF) m/z: calcd. for C₇₄H₁₂₈NNaO₆₁ [M+H+Na]²⁺, 1014.8421; found, 1014.8417.

General procedure for activation of amino oligosaccharides 49, 50, 52, 54: Each oligosaccharide was dissolved in DMF and 0.1M PBS buffer (4:1, 0.5 mL), and then disuccinimidal glutarate (15 eq) was added to the solution. The reaction was kept under gentle stirring at room temperature for 4 h, followed by removal of the solvents under vacuum. The excessive reagent was removed from the reaction by precipitation with 9 volumes of EtOAc, and the precipitates were washed 10 times with EtOAc followed by drying under vacuum to give activated oligosaccharides 55-58.

General procedure for conjugating activated oligosaccharides 55-58 with KLH and HSA: After solutions of 55-58 and KLH or HSA in a molar ratio of 30:1 in 0.1 M PBS buffer (0.35 mL) were gently stirred at room temperature for 3 days, they were applied to a Biogel A0.5 column to separate glycoconjgates 30-37 from unreacted oligosaccharides sing 0.1 M PBS buffer (I=0.1, pH=7.8) as the eluent. Fractions containing glycoconjugates, which were confirmed by the bicinchoninic acid (BCA) assay for protein and the phenol-sulfuric acid assay for carbohydrate, were combined and dialyzed against distilled water for 2 days. The solutions were lyophilized to get 30-37 as white solids.

Analysis of the carbohydrate loadings of glycoconjugates 30-37: Aliquots of a standard D-glucose solution (1 mg/mL) in water were added in ten dry 10-mL test tubes in 5 μL increment to form standard samples that contained 0 to 50 μg of glucose. In another test tube, an accurately weighed sample of the to-be-analyzed glycoconjugate 30-37 was placed. The content of glucose in each tested sample was estimated to be also in the range of 0 to 50 μg. To all of the tubes were then added 500 μL of 4% phenol and 2.5 mL of 96% sulfuric acid at room temperature. About 20 min later, the resultant colored solutions were transferred into cuvettes, and their absorptions at 490 nm wavelength (A490) were measured. A sugar calibration curve was created by plotting the A490 values of standard samples against their glucose contents (in μg), which was employed to calculate the glucose content of each tested glycoconjugate sample based on its A490 value. The carbohydrate loading of each glycoconjugate was calculated according to the following equation.

Carbohydrate loading %=sugar weight in a tested sample/total weight of the sample×100%

Immunization of mouse: After each KLH glycoconjugate 30-33 (2.17 mg of 1, 2.32 mg of 3, 2.40 mg of 5 or 1.98 mg of 7) was dissolved in 0.3 mL of 10×PBS buffer, it was diluted with water to form a 2×PBS solution (1.5 mL). The solution was well mixed with 1.5 mL of Titermax Gold adjuvant (1:1, v/v) to form an emulsion according to the protocols given by the manufacturer. Each group of six female C57BL/6J mice were initially immunized (day 1) by i.m. injection of 0.1 mL of the emulsion described above. Following the initial immunization, mice were boosted 4 times on days 14, 21, 28, and 38 by s.c. injection of the same conjugate emulsion. Therefore, each injected dose of glycoconjugate contained about 6 μg of the carbohydrate antigen. Mouse blood samples were collected through the leg veins of each mouse on day 0 prior to the initial immunization and on days 27, 38 and 48 after the boost immunizations. Finally, antisera were obtained from the clotted blood samples and stored at −80° C. before use.

The ELISA protocol: Each well of ELISA plates was treated with 100 μl of a solution of an individual HSA conjugate 34, 35, 36 or 37 (2 μg/ml) dissolved in coating buffer (0.1 M bicarbonate, pH 9.6) at 4° C. overnight and then at 37° C. for 1 h, which was followed by washing (3 times) with PBS buffer containing 0.05% Tween-20 (PBST) and treatment with blocking buffer (10% BSA in PBS buffer containing NaN3) at room temperature for 1 h. After 3 times of washing with PBST, half-log serially diluted solutions (from 1:300 to 1:656100) of a pooled or an individual mouse antiserum in PBS were added to the coated ELISA plates (100 μL/well), followed by incubation at 37° C. for 2 h. The plates were then washed with PBST and incubated at room temperature for another 1 h with a 1:1000 diluted solution of alkaline phosphatase (AP)-linked goat anti-mouse kappa, IgG1, IgG2a or IgM antibody (100 μL/well), respectively. Finally, the plates were washed with PBST and developed with 100 μL of p-nitrophenylphosphate (PNPP) solution (1.67 mg/mL in buffer) for 30 min at room temperature, which was followed by colorimetric readout at 405 nm wavelength using a microplate reader. The optical density (OD) values were plotted against the logarithmic scale of antiserum dilution values, and a best-fit line was obtained. The equation of the line was employed to calculate the dilution value at which an OD of 0.2 was achieved, and the antibody titer was obtained as the inverse of the dilution value.

In vivo evaluation of the new vaccine 31 to elicit protections against fungal infection: A group of 11 female C57BL/6J mice were immunized with an emulsion of conjugate 31 (containing 6 μg of octasaccharide antigen per dose) and Titermax Gold adjuvant prepared according to the protocol described above or with PBS (control group) on days 1, 14, 21, and 28. Thereafter, C. albicans (strain SC5314) cells (7.5×105 per mouse) in 200 μL of PBS were injected in the mice by i.v. administration on day 38. C. albican cells used in this experiment were cultured in YEPD medium at 28° C. for 24 h, and before injection, they were centrifuged and washed 3 times with PBS. The mice were checked on a daily basis, and the observation continued for 32 days after the injection of C. albican cells. Note: Animal protocols for the immunization and fungal challenge experiments were approved by the Institutional Animal Use and Care Committees of Wayne State University and Second Military Medical University.

### Methods for Synthesis of Branched β-Glucan Oligosaccharides and Immunological Studies

Compound 41. To a stirred solution of 40 (7.88 g, 12.73 mmol) in CH₂Cl₂ (400 mL) and water (22 mL) was added DDQ (5.78 g, 25.47 mmol) at room temperature (rt). After the reaction was stirred at rt for 8 h, saturated aq. NaHCO₃ solution was added, and the mixture was extracted with CH₂Cl₂. The extracts were washed with saturated aq. Na_{H}CO₃ solution and dried over Na₂SO₄. After evaporation of the solvent in vacuum, the product was purified by silica gel column chromatography (toluene/ethyl acetate 15:1 to 10:1) to give 41 (5.48 g, 90% yield) as a white solid.

Compound 59. To a solution of 41 (10.00 g, 20.09 mmol), TEA (17.9 mL, 127.27 mmol) and catalytic amount of DMAP in anhydrous CH₂Cl₂ (160 mL) was added benzoyl chloride (3.7 mL, 31.37 mmol) at 0° C. After being stirred for 12 h, the reaction mixture was washed with saturated aq. NaHCO₃ solution and brine, dried over Na₂SO₄, and concentrated under vacuum. The residue was purified by silica gel column chromatography (ethyl acetate/toluene 1:20) to afford 59 (11.44 g, 94%) as a white solid.

Compound 60. After a mixture of 59 (3.50 g, 6.00 mmol) and 4 Å MS (8 g) in anhydrous THF (120 mL) was stirred at rt for 1 h and then cooled to −40° C., BH₃.THF (29.7 mL, 30.00 mmol; 1 M solution in THF) was added. The mixture was stirred for 15 min, followed by the addition of TMSOTf (1.41 mL, 7.80 mmol) and stirring at −40° C. for another hour. The reaction mixture was slowly warmed to rt and stirred for 24 h. Then, saturated aq. NaHCO₃ solution was added at 0° C., and the mixture was diluted with CH₂Cl₂ and filtrated to remove insoluble materials. The organic layer was washed with saturated aq. NaHCO₃ solution and brine, dried over Na₂SO₄, and concentrated under reduced pressure. The residue was purified by column chromatography (ethyl acetate/toluene 1:25) to produce 60 (3.26 g, 93%) as a white solid.

Compound 70. Compound 70 (1.84 g, 92%) was prepared from 40 (2.00 g, 3.23 mmol) by the same procedure described for 60.

Compound 65. A mixture of 70 (3.72 g, 6.00 mmol), levulinic acid (0.84 g, 7.23 mmol) and EDC.HCl (1.38 g, 7.20 mmol) in CH₂Cl₂ (50 mL) was stirred at rt for 4 h. The reaction mixture was washed with water and brine, dried over Na₂SO₄ and concentrated under vacuum. The residue was dissolved in a solution of CH₂Cl₂ (100 mL) and water (1.5 mL) at rt and then DDQ (2.72 g, 12.00 mmol) was added. After being stirred at rt for 6 h, the mixture was washed with saturated aq. NaHCO₃ solution and brine, dried over Na₂SO₄ and concentrated under reduced pressure. The residue was purified by silica gel column chromatography (ethyl acetate/toluene 1:10) to produce 65 (3.10 g, 89%) as a foamy solid.

Compound 62. After a mixture of glycosyl donor 59 (300.0 mg, 0.52 mmol) and 4 Å MS (1.50 g) in anhydrous CH2Cl2 (10 mL) was stirred at rt for 1 h and then cooled to −78° C., AgOTf (397.0 mg, 1.55 mmol in 3 mL dry acetonitrile) was added, followed by p-TolSCl (74 μL, 0.52 mmol) addition using a micro-syringe 10 min later. The mixture was stirred at −78° C. for another 15 min, when TLC showed that κ was completely consumed. A solution of acceptor 41 (221.8 mg, 0.46 mmol) and TTBP (127.9 mg, 0.52 mmol) in CH₂Cl₂ (3 mL) was added. The mixture was stirred at −78° C. for 20 min and warmed to rt, followed by filtration to remove 4 Å MS. The filtrate was washed with saturated aq. NaHCO₃ solution and brine, dried over Na₂SO₄, and concentrated under vacuum. The residue was purified by silica gel column chromatography (ethyl acetate/toluene 1:30) to produce 62 (411.4 mg, 95%).

Compound 61. After a mixture of donor 59 (349.8 mg, 0.60 mmol) and activated 4 Å MS in CH₂Cl₂ (8 mL) was stirred at rt for 1 h and then cooled to −78° C., AgOTf (462.5 mg, 1.80 mmol in 1.5 mL dry acetonitrile) was added, followed by p-TolSCl (86 μL, 0.60 mmol) addition using a micro-syringe 10 min later. The mixture was stirred for another 15 min when TLC showed that 59 was completely consumed. Then, a solution of acceptor 60 (316.2 mg, 0.54 mmol) and TTBP (122.1 mg, 0.54 mmol) in CH₂Cl₂ (2 mL) was added. The mixture was allowed to warm to rt slowly over 1 h and stirred at rt for another 20 min. The mixture was then cooled to −78° C. to perform another round of glycosylation with 60 (288.1 mg, 0.49 mmol) as the glycosyl acceptor by the same protocol, which was followed by the third round of glycosylation also with 60 (262.3 mg, 0.45 mmol) as glycosyl acceptor. Finally, the reaction mixture was warmed to rt, stirred for 20 min, and then quenched with saturated aq. NaHCO₃ solution. The mixture was filtered to remove insoluble materials, and the organic layer was washed with saturated aq. NaHCO₃ solution and brine, dried over Na2SO₄ and concentrated under reduced pressure. The residue was purified by silica gel column chromatography (ethyl acetate/toluene 1:12) to give 61 (395.7 mg, 45%) as a white solid.

Compound 63. It (415.3 mg, 43%) was prepared from 59 (475.0 mg, 0.82 mmol) and 41 (1st glycosylation: 341.5 mg, 0.72 mmol; 2nd glycosylation: 310.8 mg, 0.65 mmol; 3rd glycosylation: 282.8 mg, 0.59 mmol) after 3 rounds of glycosylation reactions by the protocol described for 61 and was purified by silica gel column chromatography (ethyl acetate/toluene 1:12).

Compound 71. It (312.5 mg, 42%) was prepared from 40 (475.0 mg, 0.82 mmol) and 41 (1st glycosylation: 258.4 mg, 0.54 mmol; 2nd glycosylation: 235.2 mg, 0.49 mmol; 3rd glycosylation: 214.4 mg, 0.45 mmol) after 3 rounds of glycosylation reactions by the same protocol described for 61, which was purified by silica gel column chromatography (ethyl acetate/toluene 1:12).

Compound 64. Glycosylation of azidoethanol (15.8 mg, 0.18 mmol) with 71 (305.5 mg, 0.18 mmol) by the protocol described for 62 afforded a crude trisaccharyl glycoside intermediate that was directly dissolved in CH₂Cl₂ (10 mL) and water (0.5 mL) and treated with DDQ (82.5 mg, 0.36 mmol). After the reaction mixture was stirred at rt for 6 h, it was washed with saturated aq. NaHCO₃ solution and brine, dried over Na₂SO₄ and concentrated under reduced pressure. The residue was purified by silica gel column chromatography (ethyl acetate/toluene 1:8) to produce 64 (213.6 mg, 78%).

Compound 72. After a mixture of 63 (329.1 mg, 0.20 mmol) and activated 4 Å MS in CH₂Cl₂ (4 mL) was stirred at rt for 1 h and then cooled to −78° C., AgOTf (154.2 mg, 0.60 mmol in 1.5 mL dry acetonitrile) was added, followed by addition of p-TolSCl (29 μL, 0.20 mmol) via a micro-syringe 10 min later. The mixture was stirred for another 15 min, when TLC indicated that donor 6 was completely consumed. A solution of 65 (104.2 mg, 0.18 mmol) and TTBP (44.7 mg, 0.18 mmol) in CH₂Cl₂ (1.5 mL) was added, and the mixture was warmed to rt slowly over 1 h. After stirring at rt for another 20 min, the mixture was cooled to −78° C. to perform glycosylation with 64 (246.4 mg, 0.16 mmol) by the same protocol using AgOTf (138.7 mg, 0.54 mmol in acetonitrile 1 mL), p-TolSCl (26 μL, 0.18 mmol), and TTBP (40.7 mg, 0.16 mmol). The reaction was finally quenched with saturated aq. NaHCO₃ solution, and filtered to remove insoluble materials. The organic layer was washed with saturated aq. NaHCO₃ solution and brine, dried over Na₂SO₄, and concentrated under reduced pressure. The residue was purified by silica gel column chromatography (ethyl acetate/toluene 1:10) to give 72 (455.3 mg, 80%).

Compound 66. A mixture of 72 (420.0 mg, 120.7 μmol) and 10 mL of 0.5 M hydrazine solution in pyridine-acetic acid (4:1) buffer was stirred under an Ar atmosphere at rt for 1 h. Then 2,4-pentanedione (1 ml) was added, and the stirring continued for another 20 min. The mixture was diluted with CH₂Cl₂, washed sequentially with saturated aq. NaHCO₃, CuSO₄ and NH₄Cl solutions, dried over Na₂SO₄, and concentrated under vacuum. The residue was purified by silica gel column chromatography (ethyl acetate/toluene 1:8) to give 66 (380.6 mg, 93%) as a white foamy solid.

Compound 73. It (155.0 mg, 83%) was prepared from 61 (77.4 mg, 39.4 μmol) and 66 (120.0 mg, 35.5 μmol) by the same protocol described for 62 and was purified by silica gel column chromatography (ethyl acetate/toluene 1:12).

Compound 74. It (126.6 mg, 85%) was prepared from 62 (36.9 mg, 39.4 μmol) and 66 (120.0 mg, 35.5 μmol) by the protocol described for 62 and was purified by silica gel column chromatography (ethyl acetate/toluene 1:15).

Compound 75. It (135.8 mg, 78%) was prepared from 63 (64.8 mg, 39.4 μmol) and 66 (120.0 mg, 35.5 μmol) by the same protocol described for 62 and was purified by silica gel column chromatography (ethyl acetate/toluene 1:12).

Compound 76. To a solution of 73 (20.0 mg, 3.8 μmol) in CH₂Cl₂ (4 mL) was added acetic acid (4 drops) and zinc powder (20 mg). After vigorously stirring at rt for 24 h, the mixture was filtered through a Celite pad and concentrated under vacuum. The residue was dissolved in AcOH and H₂O (5:1, 15 mL) and heated at 60° C. for 24 h. The solvents were removed in vacuum and co-evaporated with toluene 5 times. The resulting residue was dissolved in t-BuOH and H₂O (4:1, 15 mL), and NaOH (15 mg in 1.5 mL H₂O) was added in portions. After the mixture was heated at 40° C. for 24 h, the solvents were removed by lyophilization. The residue was dissolved in water and neutralized with 0.25 N HCl, and then lyophilized to give the crude product that was purified on a Sephadex G-25 gel filtration column with water as the eluent. Lyophilization gave 76 (7.1 mg, 87%) as a white fluffy solid. ¹H NMR (600 MHz, D₂O) δ: 4.58 (m, 8H), 4.39-4.33 (m, 5H), 4.08-4.02 (m, 4H), 3.97-3.94 (m, 1H), 3.77 (m, 10H), 3.69 (m, 5H), 3.65-3.50 (m, 18H), 3.50-3.26 (m, 34H), 3.26-3.11 (m, 9H), 3.10 (t, J=4.8 Hz, 1H). HRMS (ESI TOF): calcd. for C₈₀H₁₃₈NNaO₆₆ [M+H+Na]²⁺ m/z, 1095.8686; found, 1095.8658.

Compound 77. It (5.8 mg, 92%) was prepared from 74 (15.0 mg, 3.6 μmol) by the same protocol described for 76. ¹H NMR (600 MHz, D₂O) δ: 4.58 (m, 9H), 4.40 (d, J=8.2 Hz, 2H), 4.06 (d, J=10.7 Hz, 1H), 4.00-3.95 (m, 1H), 3.83-3.71 (m, 12H), 3.58 (m, 20H), 3.41 (m, 30H), 3.26-3.23 (m, 2H), 3.22-3.18 (m, 2H), 3.12 (d, J=4.7 Hz, 2H). HRMS (ESI TOF): calcd. for C₆₈H₁₁₈NO₅₆ [M+H] m/z, 1844.6416; found, 1844.6464.

Compound 78. It (6.0 mg, 85%) was prepared from 75 (15.0 mg, 3.1 μmol) by the same protocol described for 76. ¹H NMR (600 MHz, D₂O) δ: 4.60 (m, 11H), 4.39 (d, J=7.8 Hz, 2H), 4.06 (d, J=12.1 Hz, 1H), 3.98-3.93 (m, 1H), 3.77 (m, 14H), 3.58 (m, 25H), 3.49-3.28 (m, 35H), 3.25 (t, J=8.0 Hz, 2H), 3.20 (t, J=8.6 Hz, 2H), 3.09 (s, 2H). HRMS (ESI TOF): calcd. for C₈₀H₁₃₈NNaO₆₆ [M+H+Na]²⁺ m/z, 1095.8686; found, 1095.8641.

Preparation of HSA/KLH-oligosaccharide conjugates: Each synthetic oligosaccharide (5.0 mg) was dissolved in a mixture of DMF and 0.1M PBS (4:1, 0.5 mL), and to the solution was added DSG (15 eq). After the mixture was stirred at rt for 4 h, solvents were removed under vacuum. The resultant activated oligosaccharides were separated from excessive DSG through precipitation with EtOAc (4.5 mL) and washing with EtOAc 10 times. The products were mixed with HSA or KLH (in 30:1 molar ratio) in 0.1M PBS (0.35 mL) with stirring at rt for 3 days. The reaction mixtures were applied to a Biogel A0.5 column to remove excessive oligosaccharides with 0.1M PBS buffer (I 0.1, pH 7.8) as eluent. Fractions containing the glycoconjugates were combined and dialyzed against distilled water for 2 days. The solution was finally lyophilized to afford the glycoconjugates 82-87 as white fluffy solids.

Analysis of carbohydrate loadings of the glycoconjugates: Aliquots of a standard D-glucose solution (1 mg/mL) in water were added in ten dry 10-mL test tubes in 5 μL increment to give standard samples containing 0 to 50 μg of glucose. Meanwhile, accurately weighed samples of the to-be-analyzed glycoconjugate (82-87, with the estimated glucose content in 0 to 50 μg range) and the corresponding protein were added in two other tubes. To the tubes were added 4% phenol (500 μL) and 96% sulfuric acid (2.5 mL). After 20 min of stirring, these solutions were transferred into cuvettes, and their absorptions at 490 nm wavelength (A490) were measured. A sugar calibration curve was created by plotting the A490 of standard samples against the glucose contents, and was utilized to calculate glucose content of each tested glycoconjugate based on its A490 after subtracting the A490 of corresponding protein sample:

Carbohydrate loading (%)=sugar weight in a tested sample/total weight of the sample×100%.

Immunization of mouse: Each glycoconjugate 82-84 (2.07, 2.36 and 2.07 mg, respectively) was dissolved in 10×PBS (0.3 mL) and then diluted with water to form 2×PBS solution. It was mixed with CFA (1:1, v/v, 1.5 mL) according to the manufacturer's protocol to form an emulsion. Each group of five female C57BL/6J mice (Jackson Laboratory) were initially immunized (day 1) via i.m. injection of an emulsion (0.1 mL) containing about 6 μg of the carbohydrate antigen. Thereafter, each mouse was boosted four times on days 14, 21, 28, and 38 by s.c. injection of the same emulsion. Mouse blood samples were collected via mouse leg veins on day 0 prior to initial immunization and on days 27, 38 and 48 after boost immunizations. Antisera were prepared from the clotted blood samples.

ELISA assay. ELISA plates were treated with a solution (100 μl) of HSA conjugate 85-87 (2 μg/ml) dissolved in coating buffer (0.1M bicarbonate, pH 9.6) at 4° C. overnight. The plates were incubated at 37° C. for 1 h, washed three times with PBS containing 0.05% Tween-20 (PBST), and incubated with blocking buffer containing 1.0% bovine serum albumin (BSA) in PBS at rt for 1 h. After washing with PBST three times, to the plates was added three-fold diluted (from 1:300 to 1:656100) antiserum in PBS (100 μL/well), followed by incubation at 37° C. for 2 h. The plates were washed with PBST and incubated at rt for 1 h with 1:1000 diluted solutions of alkaline phosphatase-linked goat anti-mouse kappa, IgG1, IgG2b, IgG2c, IgG3 or IgM antibody (100 μL/well). The plates were developed with p-nitrophenylphosphate (PNPP) (1.67 mg/mL, 100 μL) for 30 min at rt and analyzed at 405 nm wavelength. The observed optical density (OD) was plotted against antiserum dilution values in logarithmic scale, and the best-fit line was used to calculate antibody titers that were defined as the dilution value at an OD value of 0.2.

Assay of Lam inhibition on antiserum binding to the synthetic oligosaccharides. ELISA plates were coated with HSA conjugates 85-87 (2 μg/ml) dissolved in 0.1M coating buffer at 37° C. for 1 h. After being washed with PBST 3 times, the plates were incubated with BSA blocking buffer. The pooled antisera (1:900 dilution) were mixed with serially diluted PBS solutions of Lam (from 0.01 to 200 μg/ml), and the mixtures were added to the plates that were incubated at 37° C. for 2 h, washed, and incubated with 1:1000 diluted solution of AP-labeled goat anti-mouse kappa antibody (100 μL/well) at rt for 1 h. The plates were washed, developed with PNPP (1.67 mg/mL, 100 μL) at rt for 30 min, and analyzed at 405 nm wavelength.

% inhibition of binding=(Aw/o−Aw)/Aw×100%, where Aw/o is the absorbance without Lam and Aw is the absorbance in the presence of Lam.

Immunofluorescence assay. HKCA cells were smeared on IF microscope slides that were dried, washed with PBST, and treated with 3% BSA blocking buffer at 37° C. for 1 h. The slides were incubated with 1:3 diluted (in PBST) antiserum or normal serum at 37° C. for 2 h, followed by washing and incubation with FITC-labeled goat anti-mouse kappa at rt for 1 h. The slides were washed, mounted with the Fluoromount aqueous mounting medium, and studied with the Zeiss ApoTome Imaging System using 100×/1.30 Oil objective lens.

In vivo evaluation of 82 and 84 to protect mice against C. albicans infection: Each group of 11 female C57BL/6J mice were immunized with an emulsion of 82 or 84 (6 μg carbohydrate antigen per dose) or with PBS (control) on days 1, 14, 21, and 28. Then, C. albicans (strain SC5314) cells (7.5×105/mouse), harvested from pre-cultured YEPD medium at 28° C. for 24 h, in 200 μL PBS were i.v. injected in the mice on day 38. The mice were monitored daily for 30 days after the systemic challenge with C. albican cell.

Experimental Procedures for the Synthesis of Haemophilus influenzae Type B Carbohydrates and Immune Response Studies

Compound 99. To a stirred solution of D-ribose 98 (8.0 g, 53.30 mmol) in pyridine (100 ml) was added triphenylmethyl chloride (16.4 g, 58.82 mmol) at rt. The reaction mixture was stirred for 48 h. After removing most of pyridine under vacuum, the residue was poured into ice-water, and the mixture was extracted with DCM. The organic layer was washed with brine, dried over anhydrous Na2SO4 and evaporated under reduced pressure. The residue was recrystallized from ethanol to give 5-O-trityl-D-ribose as a white solid (15.7 g). This intermediate was dissolved in absolute ethanol/DCM (30 ml/125 ml) and NaBH₄ (3.15 g, 83.27 mmol) was added slowly at rt. The mixture was stirred at 25° C. for 3 h, the reaction was quenched with 10% acetic acid to bring PH at about 5. The mixture was extracted several times with DCM. The combined organic layer was washed with ice cold water, brine and dried over anhydrous Na₂SO₄, concentrated to give 99 (16.0 g) as white syrup.

General Procedure for Benzylation:

To a stirred solution of alcohol compound (1 mmol) in anhydrous DMF (2 ml) under Ar atmosphere was added NaH (1.5 mmol), stirred for 30 min followed by dropwise addition of BnBr (1.5 mmol) and stirred at rt for 6 h. The reaction mixture was quenched with methanol and diluted with DCM. The organic layer was washed with water and brine, dried over anhydrous Na₂SO₄ and concentrated under vacuum. The residue was purified by silica gel chromatography to give the desired product.

Compound 100. The mixture of 99 (15.0 g, 38.0 mmol), 4,4′-Dimethoxytrityl chloride (14.1 g, 41.8 mmol), triethylamine (7.7 ml, 76.0 mmol) and DMAP (50 mg, catalytic amount) in DMF (150 ml) was stirred at rt for 24 h. The reaction mixture was diluted with DCM, washed with water, brine, dried over anhydrous Na₂SO₄ and concentrated under vacuum to give triol intermediate which on reaction with BnBr (21 ml, 171.0 mmol) and NaH (4.1 g, 171.0 mmol) by using above procedure to give crude tribenzyl protected intermediate. The crude intermediate was further dissolved in a solution of 1M formic acid in DCM (400 ml) and stirred at rt. The reaction was monitored by TLC, after completion of reaction the mixture was washed with sat. NaHCO₃, brine, dried over anhydrous Na₂SO₄ and concentrated under reduced pressure. The obtained crude material was purified by silica gel column chromatography to give 100 (17.6 g, 70.0%) as colorless oil.

Compound 101. A crude p-methoxybenzyl-protected intermediate was obtained from 100 (12.0 g, 18.1 mmol) by using the similar procedure used for benzyl protection (instead of benzyl bromide use PMB-chloride). The obtained PMB protected intermediate was dissolved in a mixture of formic acid (120 ml) and CH₃CN (160 ml) and stirred at 0° C. for 1 h. The reaction was quenched with sat. NaHCO₃ and extracted with DCM. The organic layer was washed with water, brine, dried over anhydrous Na₂SO₄, and concentrated under vacuum to give crude material, which was further purified by silica gel column chromatography to obtain 101 (8.4 g, 85.5%) as colorless oil.

Compound 102. A crude allyl-protected intermediate was obtained from 101 (8.0 g, 14.7 mmol) by using the similar procedure of benzyl protection, (instead of benzyl bromide use allyl chloride). A solution of this intermediate in 100 ml of 10% TFA in DCM was stirred at rt for 1 h. The reaction was quenched with sat. NaHCO₃ and extracted with DCM. The organic layer was washed with water, brine, dried over anhydrous Na₂SO₄, concentrated, and purified by silica gel column chromatography to give 102 (6.0 g, 88.2%) as colorless oil.

Compound 103. To a stirred solution of D-ribose 98 (15.0 g, 100.0 mmol), 2,2-dimethoxy propane (DMP) (30.0 ml, 244.8 mmol), methanol (21.0 ml, 514.8 mmol) in acetone (120 ml) was added perchloric acid (6 ml) at 0° C. and reaction mixture was continued to stir at rt for 2 h. After completion of reaction the mixture was quenched with sat. NaHCO₃. A solid precipitated was removed by filtration and the filtrate was concentrated under reduced pressure. The obtained crude residue was dissolved in DCM, washed with water, brine, dried over anhydrous Na₂SO₄ and concentrated under vacuum. The crude was purified by high vacuum distillation to give colorless oil intermediate (14.8 g). The obtained intermediate (8.8 g, 43.2 mmol) was then subjected for benzyl protection by using above procedure to give 103 (11.5 g, 90.5%).

Compound 104. A solution of 103 (8.8 g, 30.0 mmol) in methanol (100 ml) and 10 ml of 0.5M aq. HCl was refluxed for 3 h, cooled to rt, neutralized with NaHCO₃, and concentrated under vacuum. The crude residue was co-evaporated with pyridine two times, then dissolved in pyridine (100 ml) and benzoyl chloride (9.1 ml, 78.0 mmol) was added dropwise at 0° C. and reaction mixture was allowed to stir at rt for 6 h. The reaction mixture was quenched with aq. NaHCO₃, extracted with DCM, washed with water, brine, dried over anhydrous Na₂SO₄ and concentrated under reduced pressure. The residue was purified by silica gel column chromatography to give 104 (10.0 g, 72.1%) as a syrup.

Compound 105. A solution of 104 (8.0 g, 17.3 mmol) in dioxane (80 ml) and 2 M aq. HCl (80 ml) was refluxed for 4 h, cooled to rt, extracted with DCM, washed with sat. NaHCO₃, water, brine, dried over anhydrous Na₂SO₄ and concentrated under vacuum. The residue was purified by silica gel column chromatography to give 105 (5.3 g, 68.3%) as a mixture of α and β isomers.

Compound 106. To a stirred solution of 105 (5.0 g, 11.1 mmol), CCl₃CN (5.6 ml, 55.7 mmol) in dry DCM (50 ml) was slowly added DBU (0.3 ml, 2.2 mmol) under an Ar atmosphere at 0° C. After 1.5 h, the reaction mixture was concentrated under vacuum and purified with a triethylamine-neutralized silica gel column chromatography to give trichloroacetimidte 106 (6.1 g, 93.0%) as a mixture of α and β (α:β=100:18).

Compound 107. A mixture of trichloroacetimidate 106 (5.0 g, 8.4 mmol), acceptor 102 (3.7 g, 8.0 mmol) and MS 4 Å (5.0 g) in anhydrous DCM (30 ml) was stirred under Ar atmosphere at rt for 1 h. After cooling to 0° C., TMSOTf (0.3 ml, 1.6 mmol) was added and the reaction was stirred for 20 min. Neutralization with triethylamine was followed by filtration through Celite, concentration under vacuum and purification by column chromatography gave 107 (6.8 g, 95.0%) as a colorless oil.

Compound 108. To a stirred solution of 107 (6.5 g, 7.3 mmol) in dry methanol (100 ml) was slowly added NaOMe (131.4 mg, 2.4 mmol) at rt. After 2 h, the reaction mixture was neutralized with amberlyst resin, filtered and filtrate was concentrated under vacuum and purified by column chromatography to give diol 108 (5.0 g) quantitively.

Compound 109. A mixture of diol 108 (4.2 g, 6.1 mmol) and dibutyltin oxide (1.8 g, 7.4 mmol) in anhydrous methanol (50 ml) was refluxed for 6 h. After cooling to rt the reaction mixture was concentrated under vacuum. To the solution of crude residue in anhydrous DMF (40 ml) at 0° C. was added CsF (1.4 g, 9.2 mmol) and BnBr (2.9 ml, 24.5 mmol and stirred for 24 h at rt. The reaction mixture was filtered through Celite into sat. NaHCO₃ and EtOAc. The organic layer was washed with water, brine, dried over anhydrous Na₂SO₄ and concentrated under vacuum. The residue was purified by silica gel column chromatography to give 109 (3.3 g, 70.0%) as a colorless oil.

Compound 110. To a stirred solution of 109 (600.0 mg, 0.78 mmol), levulinic acid (300.0 mg, 2.58 mmol), DMAP (10.0 mg, catalytic amount) in DCM (10 ml) was added EDC. HCl (222.3 mg, 1.16 mmol) under Ar atmosphere at 0° C. and stirred at rt for 8 h. The reaction mixture was washed with water, sat.NaHCO₃, brine, and dried over anhydrous Na₂SO₄. Evaporation of the solvent followed by purification by silica gel column chromatography gave 110 (640 mg, 94.0%) as a colorless oil.

General Procedure for Deallylation:

A solution of 1,5-cyclopentadiene-bis(methyldiphenylphosphine) iridium hexafluoropho-sphate (0.05 mmol) in anhydrous THF (5 ml) was stirred under H₂ atmosphere at rt until the color changed from red to yellow. The solution was degassed by purging Ar gas for 15 min. Allyl-protected compound (1 mmol) in 5 ml THF was added to the above solution and stirred at rt for 2 h. The reaction was concentrated under vacuum, then dissolved in acetone-water (9:1, 6 ml) and treated with HgCl₂ (5 mmol) and HgO (0.05 mmol). The reaction mixture was stirred at rt for 2 h, then directly concentrated under vacuum and purified by silica gel or sephadex LH-20 column chromatography to offer the deprotected product.

Compound 111. It (570.3 mg, 96.4%) was obtained using the procedure for deallylation from 110 (620.3 mg, 0.71 mmol) after silica gel column chromatography purification.

General Procedure for H-Phosphonation:

Alcohol Compound (1 mmol) and phosphonic acid (2.5 mmol) were co-evaporated with dry pyridine three time and then dissolved in dry pyridine (3 mL) and the solution was added pivaloyl chloride (2.5 mmol) in pyridine (2 mL) at rt. After 6 h, the reaction solution was concentrated under vacuum. The residue was purified by triethylamine-neutralized silica gel column chromatography to give H-phosphonate compound.

Compound 112. It (3.3 g, 80.3%) was obtained from 109 (3.8 g, 4.9 mmol) by using the procedure for H-phosphonation (silica gel column purification).

Compound 113. It (2.7 g, 85.7%) was obtained from 108 (2.5 g, 3.7 mmol) by using the same procedure of benzylation.

Compound 114. It (2.3 g, 90.0%) was obtained from 113 (2.7 g, 3.1 mmol) by using the same procedure of deallylation (silica gel column purification).

General Procedure for Synthesis of Phosphadiester:

Alcohol compound (1 mmol) and H-phosphonate compound (1.5 mmol) were co-evaporated with dry pyridine three time and then dissolved in dry pyridine (10 mL). To the stirred solution was added pivaloyl chloride (3.0 mmol) in dry pyridine (5 mL) under an Ar atmosphere at rt. After 6 h, the reaction mixture was cooled to 0° C. and I₂ (1.5 mmol) in 1 ml of pyridine and water (10:1, V/V) was added and stirred for 3 h at rt, quenched by sat. NaS₂O₃, extracted with CHCl₃, dried over anhydrous Na₂SO₄ and concentrated under vacuum. The residue was purified by triethylamine-neutralized silica gel or sephadex LH-20 column chromatography to give the phosphate product.

Compound 115. It (452.2 mg, 85.0%) was obtained from 112 (402.7 mg, 0.48 mmol) and 114 (264.0 mg, 0.32 mmol) by using the similar procedure of phosphadiester (silica gel column purification).

Compound 116. It (377.8 mg, 93.1%) was obtained from 115 (415.0 mg, 0.25 mmol) by using the same procedure of deallylation (silica gel column purification).

Compound 117. It (319.7 mg, 79.8%) was obtained from 112 (205.0 mg, 244 μmol) and 116 (264.0 mg, 163 μmol) by using the same procedure of synthesis of phosphadiester (sephadex LH-20 column purification).

Compound 118. It (257.3 mg, 87.2%) was obtained from 117 (300.0 mg, 122 μmol) by using same procedure of deallylation (sephadex LH-20 column purification).

Compound 119. It (156.3 mg, 77.5%) was obtained from 112 (78.0 mg, 93 μmol) and 118 (150.0 mg, 62 μmol) by using the same procedure of synthesis of phosphadiester (sephadex LH-20 column purification).

Compound 120. It (97.8 mg, 82.5%) was obtained from 119 (120.0 mg, 37 μmol) by using the same procedure of deallylation (sephadex LH-20 column purification).

Compound 121. To a stirred solution of D-ribose (15.0 g, 100 mmol) in 150 mL of anhydrous pyridine was slowly added acetic anhydride (46.2 ml, 489.7 mmol) at 0° C. After stirring for 24 h, the mixture was concentrated under vacuum and the residue was extracted with EtOAc and ice cold water, organic layer washed with sat. NaHCO₃, brine, dried over anhydrous Na₂SO₄ and evaporated to give a white solid intermediate (30.0 g). To the stirred mixture of this intermediate (10 g, 31.4 mmol), 2-azidoethanol (5.5 g, 62.8 mmol) and MS 4 Å (3.5 g) in anhydrous DCM (40 ml) under Ar, BF₃.Et₂O (6.1 ml, 0.047 mol) was added dropwise at 0° C. After stirring at rt for 1 day, the reaction was quenched with sat. NaHCO₃, diluted with DCM and filtered through a Celite pad. The filtrate was washed with brine, dried over Na₂SO₄ and concentrated under vacuum. The residue was purified by silica gel column chromatography to give 121 (7.7 g, 71.0%) as colorless oil.

Compound 122. To a stirred solution of 121 (4.4 g, 17.7 mmol) in dry methanol (100 ml) was added NaOMe (234.9 mg, 4.35 mmol) at rt. After 1 h, the reaction mixture was neutralized with amberlyst resign and filtered. The filtrate was concentrated under vacuum to give triol intermediate (2.5 g) as a solid. A mixture of this intermediate (2.2 g, 10.0 mmol) and dibutyltin oxide (3.0 g, 12.0 mmol) in anhydrous methanol (40 ml) was refluxed for 6 h, cooled to rt and concentrated under vacuum. The residue was dissolved in a mixture of anhydrous DMF (20 ml) and toluene (10 ml), then NaH (264.0 mg, 11.0 mmol), TBAl (3.7 g, 10.0 mmol) was added at rt. After stirring for 30 min, BnBr (3.6 ml, 30.0 mmol) was added and the reaction mixture was stirred under Ar atmosphere at rt for 24 h. The reaction mixture was diluted with EtOAc, washed with sat. NaHCO₃, brine, dried over anhydrous Na₂SO₄, and concentrated under vacuum. The residue was purified by silica gel column chromatography to give 122 (3.2 g, 80.1%) as a colorless oil.

Compound 123. It (2.7 g, 77.8%) was obtained from 122 (3.0 g, 7.5 mmol) by using the same procedure of H-phosphonation (silica gel column purification).

Compound 124. It (509.4 mg, 81.8%) was obtained from 111 (400.0 mg, 481 μmol) and 123 (288.1 mg, 721 μmol) by using the same procedure of the synthesis of phosphadiester (silica gel column purification).

Compound 125. A solution of 124 (500.0 mg, 386 μmol) in 10 ml of 0.5 M hydrazine in pyridine-acetic acid (4:1) buffer was stirred under an Ar atmosphere at rt for 1 h and 2,4-pentanedione (1 ml) was added. After 20 min, the mixture was diluted with CHCl₃, washed with sat. NaHCO₃, sat. CuSO₄, and sat. NH₄Cl, dried over anhydrous Na₂SO₄ and concentrated under vacuum. The residue was purified by sephadex LH-20 column chromatography to give a white foam intermediate (460.5 mg). Obtained intermediate (460.5 mg, 385 μmol) was transformed to compound 125 (415.5 mg, 85.6%) by using the same procedure of H-phosphonation (sephadex LH-20 column purification).

Compound 126. It (87.1 mg, 79.3%) was obtained from 116 (74.1 mg, 46 μmol) and 125 (48.0 mg, 38 μmol) by using the procedure of synthesis of phosphadiester (silica gel column purification).

Compound 127. It (81.8 mg, 89.0%) was obtained from 118 (61.1 mg, 25 μmol) and 125 (35.0 mg, 28 μmol) by using the same procedure of synthesis of phosphadiester (silica gel column purification).

Compound 128. It (101.0 mg, 80.6%) was obtained from 122 (90.0 mg, 28 μmol) and 125 (38.8 mg, 31 μmol) by using the same procedure of synthesis of phosphadiester (silica gel column purification).

Compound 129. A mixture of 126 (40.0 mg, 14 μmol) and 10% Pd-C (20.0 mg) in MeOH:H₂O (4:1, 10 ml) was shaken under hydrogen at 50 psi for 48 h. The catalyst was removed by filtration through a Celite pad and the pad was subsequently washed with MeOH:H₂O (1:1). The combined filtrate was concentrated under vacuum and the residue was dissolved in 2 ml of H₂O and lyophilized to give 129 (18.4 mg) in quantitative yield as a white solid. ¹H NMR (600 MHz, D₂O) δ 4.9, 14.88, 4.85 (3s, 3H, H-1″, H-1″, H-1′″), 4.75 (s, 1H, H-1), 4.48-4.39 (m, 3H, H-3′, H-3″, H-3′″), 4.33-4.25 (m, 1H, H-3), 4.24-3.41 (m, 39H), 3.10 (m, 2H, CH₂NH₂), 3.03 (q, J=7.3 Hz, 10H, NCH₂CH₃), 1.10 (t, J=7.3 Hz, 12H, NCH₂CH₃). ³¹P NMR (161 MHz, D₂O) δ 0.70 (1P), 0.17 (2P). [α]_{D}²⁵=−226.4° (c 0.4, H₂O). HRMS (ESI): calcd. for C₃₇H₇₂NO₃₈P₃ [M+2Na-3H]⁻ m/z, 1274.2506; found, 1274.2515.

Compound 130. It (20.5 mg) was prepared from 127 following the same procedure described for 129. ¹H NMR (500 MHz, D₂O) δ4.91, 4.87 (2s, 4H, H-1′, H-1″, H-1′″, H-1″″), 4.78 (s, 1H, H-1), 4.52-4.41 (m, 4H, H-3′, H-3″, H-3′″, H-3″″), 4.36-4.29 (m, 1H, H-3), 4.16-3.46 (m, 50H), 3.17-3.09 (m, 1H, CH₂NH₂), 3.05 (q, J=7.0 Hz, 22H, NCH₂CH₃), 1.11 (t, J=7.0 Hz, 33H, NCH₂CH₃). ³¹P NMR (161 MHz, D₂O) δ 0.68 (1P), 0.15 (3P). [α]_{D}²⁵=−202.3° (c 0.5, H₂O). HRMS (ESI): calcd. for C₄₇H₉₁NO₄₉P₄ [M−H]⁻ m/z, 1576.3532; found, 1576.3561.

Compound 131. It (17.4 mg) was prepared from 128 following the same procedure described for 129. ¹H NMR (500 MHz, D₂O) δ 4.87 (s, 5H, H-1′, H-1″, H-1′″, H-1″″, H-1′″″), 4.75 (s, 1H, H-1), 4.48-4.34 (m, 5H, H-3′, H-3″, H-3′″, H-3″″, H-3′″″), 4.32-4.24 (s, 1H, H-3), 4.21-3.36 (m, 61H), 3.12-3.06 (m, 2H, CH₂NH₂), 3.00 (q, J=7.0 Hz, 24H, NCH₂CH₃), 1.09 (t, J=7.1 Hz, 36H, NCH₂CH₃). ³¹P NMR (161 MHz, D₂O) δ 0.71 (1P), 0.19 (4P). [α]_{D}²⁵=−186.4° (c 0.5, H₂O). HRMS (ESI): calcd. for 2[C₅₇H₁₀₆NO₆₀P₅ [M+2Na-4H]²] m/z, 1965.3758; found, 1965.3714.

General Procedure for Activation of Amino-Oligosaccharide:

A mixture of amino-oligosaccharide 123-125 (5 mg) and disuccinimidal glutarate (15 eq) in DMF:PBS (0.1M PBS buffer) (4:1, 0.5 ml) was stirred at rt for 4 h. The solution was concentrated under vacuum and the residue was wash with EtOAc 10 times. The solid of activated oligosaccharides was dried under vacuum for 1 h, and directly used to conjugate with HSA and KLH.

General Procedure for Conjugation with HSA and KLH:

A mixture of the activated oligosaccharides 132-134 and 5 mg of HSA or KLH in 0.4 ml of 0.1 M PBS buffer was gently stirred at rt for 3 days. The mixture was purified by Biogel A 0.5 column with 0.1 M PBS buffer as the eluent. The glycoconjugate-containing fractions indicated by the bicinchoninic acid (BCA) assay for proteins were combined and dialyzed for 1 day, and then lyophilized to give the desired glycoconjugates 135-140 as white solids.

Analysis of the Carbohydrate Loading of Glycoconjugates 135-139.

The phenol-sulfuric acid assay: Determination of sugar loading using phenol-sulfuric acid is based on the absorbance at 490 nm of a colored aromatic complex formed between phenol and the carbohydrate. The method is very general, and can be applied to reducing and nonreducing sugars and to many classes of carbohydrates including oligosaccharides. The amount of sugar present is determined by comparison with a calibration curve using a spectrophotometer. Calibration sugar standards were prepared using 1 mg/ml ribose standard solution in distilled water. Aliquots were transferred to 10 different, dry 10-ml tubes in 5-μl increments ranging from 5 to 50 μl. In another 10-ml test tube, accurately weighed sample of glycoconjugate to be analyzed were placed. At this point, all the tubes should contain between 5 to 50 μg of sugar, and one should contain an unknown amount of sugar to be determined. To all the tubes were added 500 μl of 4% phenol followed by 2.5 ml 96% sulfuric acid. All the glycosidic linkages were broken and the colored complex is formed in this step. Solutions from the test tubes were transferred to the cuvettes and measured the A490 of the sugar standards and unknown solution. To calculate the amount of sugar present in the unknown sample, a graph was plotted against A490 versus sugar weight (μg) of the sugar calibration standards. The intercept of the A490 of the unknown sample with the calibration line represents the amount (μg) of sugar present in the glycoconjugate. The carbohydrate loading of each glycoconjugate was calculated according to the following equation, and the results are shown below.

| | KLH conjugates | | | HAS conjugates | | |
|---|---|---|---|---|---|---|
| | sample | | | | | |
| | 135 | 136 | 137 | 138 | 139 | 140 |
| Loading (%) | 8.4 | 8.4 | 9.0 | 13.0 | 9.2 | 7.5 |

Compound 142. A solution of 141 (24.0 mg, 11.4 μmol), p-nitrophenol (7.8 mg, 57.0 μmol) and EDC.HCl (10.8 mg, 57.0 μmol) in dry DCM (4 mL) was stirred at 10° C. After 6 h, the reaction was diluted with DCM, washed with water, brine, dried over anhydrous Na₂SO₄ and concentrated under vacuum. The residue was purified by silica gel column chromatography to give 142 (19.1 mg, 74.8%) as a white solid.

Compound 146. A mixture of 126 (11.4 mg, 4 μmol) and lindlar catalyst (20.0 mg) in MeOH (2 ml) was shaken under hydrogen at 10 psi for 4 h. The catalyst was removed by filtration and the filtrate was concentrated under vacuum to give the crude amine 143. The crude 143 [MALDI TOF MS (positive mode): calcd. For C₁₆₃H₁₈₀NO₃₈P₃ [M+H]⁺ m/z, 2853.15; found, 2853.48] was used for the next step without further purification. The mixture of 142 (9.0 mg, 4 μmol), 143 and triethylamine (5.5 μl, 40 μmol) was stirred at rt for 2 days and concentrated under vacuum and co-evaporated with toluene a couple of times. The residue was purified by preparative TLC plate (CH2Cl2, MeOH and Et₃N; 10:1:0.1) to give 146 (10.5 mg, 50.0%) as a triethylammonium salt. ¹H NMR (400 MHz, CDCl₃) δ 7.48-7.03 (m, 110H, ArH), 6.92, 6.80, 6.71, 6.56 (4br, 4H, NH), 5.48 (m, 1H, H-3′ of lipid), 5.24-4.96 (m, 4H), 4.95-4.82 (m, 12H), 4.79-4.22 (m, 46H), 4.22-4.07 (m, 5H), 4.06-3.24 (m, 43H), 3.17-3.09 (m, 2H), 3.04-2.92 (m, 40H, NCH₂CH₃), 2.82-2.53 (m, 6H), 2.45-2.07 (m, 8H of lipid), 1.62-1.38 (m, 10H of lipid), 1.34-1.00 (m, 164H, 104H of lipid and 60H of NCH₂CH₃), 0.87 (t, J=6.4 Hz, 18H, 6CH₃, lipid). HRMS (ESI): calcd. for C₂₈₅H₃₇₆N₄O₆₀P₄ [M−2H]²⁻ m/z, 2468.2644; found, 2468.2537; calcd. for C₂₈₅H₃₇₆N₄O₆₀P₄ [M−3H]³⁻ M/Z, 1645.1737; found, 1645.1519.

Compound 147. Compound 147 (9.4 mg) as a triethylammonium salt was prepared from 144 [MALDI TOF MS (positive mode): calcd. For C₂₀₈H₂₂₉NO₄₉P₄ [M+H]⁺ m/z, 3649.45; found, 3649.18] by following the same procedure described for synthesis of 146. ¹H NMR (400 MHz, CDCl₃) δ 7.54-7.04 (m, 135H, ArH), 6.95, 6.85 (2br, 2H, NH), 6.55 (br, 2H, NH), 5.45 (m, 1H, H-3′ of lipid), 5.34-4.98 (m, 4H), 4.95-4.85 (m, 14H), 4.84-4.24 (m, 57H), 4.23-4.00 (m, 6H), 3.98-3.18 (m, 54H), 2.99-2.83 (m, 30H, NCH₂CH₃), 2.72-2.07 (m, 14H of lipid), 1.72-1.43 (m, 10H of lipid), 1.34-1.00 (m, 149H, 104H of lipid and 45H of NCH₂CH₃), 0.87 (t, J=6.4 Hz, 18H, 6CH₃, lipid). HRMS (ESI): calcd. for C₃₃₀H₄₂₅N₄O₇₁P₅ [M−2H]²⁻ m/z, 2866.4150; found, 2866.7986; calcd. for C₃₃₀H₄₂₅N₄O₇₁P₅ [M−3H]³⁻ m/z, 1910.6074; found, 1910.9133; calcd. for C₃₃₀H₄₂₅N₄O₇₁P₅ [M−4H]⁴⁻ m/z, 1432.7036; found, 1433.1818.

Compound 148. Compound 148 (10.1 mg) was prepared as a triethylammonium salt from 145 [MALDI TOF MS (positive mode): calcd. For C₂₅₃H₂₇₈NO₆₀P₅ [M+H]⁺ m/z, 4445.74; found, 4446.19] by following the same procedure as described for the synthesis of 146. ¹H NMR (400 MHz, CDCl₃) δ 7.54-7.04 (m, 160H, ArH), 6.97, 6.88, 7.76, 6.56 (4br, 4H, NH), 5.48 (m, 1H, H-3′ of lipid), 5.35-5.30 (m, 1H), 5.24-4.98 (m, 3H), 4.97-4.80 (m, 16H), 4.80-4.21 (m, 68H), 4.17-4.00 (m, 7H), 3.98-3.13 (m, 63H), 3.02-2.85 (m, 64H, NCH₂CH₃), 2.72-1.96 (m, 14H of lipid), 1.63-1.37 (m, 10H of lipid), 1.36-1.02 (m, 200H, 104H of lipid and 96H of NCH₂CH₃), 0.85 (t, J=6.4 Hz, 18H, 6CH₃, lipid). HRMS (ESI): calcd. for C₃₇₅H₄₇₄N₄O₈₂P₆ [M−2H]²⁻ m/z, 3264.5257; found, 3265.5657; calcd. for C₃₇₅H₄₇₄N₄O₈₂P₆ [M−3H]³⁻ m/z, 2176.0412; found, 2176.0413; calcd. for C₃₇₅H₄₇₄N₄O₈₂P₆ [M−4H]⁴⁻ m/z, 1631.7789; found, 1631.7789; calcd. for C₃₇₅H₄₇₄N₄O₈₂P₆ [M−5H]⁵⁻ m/z, 1305.2216; found, 1305.2216.

Compound 149. A mixture of 146 (11.0 mg, 2 μmol), 10% Pd-C (40.0 mg) in MeOH and DCM (1:1, 6 ml) was shaken under H₂ at 50 psi for 48 h. The catalyst was removed by filtration through a Celite pad, and the pad was subsequently washed with MeOH:CDM (1:1). The combined filtrate was concentrated under vacuum and the residue was purified by sephadex LH-20 to afford 149 (4.5 mg) in quantitative yield as an oil.

Compound 150. It (4.0 mg) was prepared from 147 following the same procedure described for the synthesis of 149.

Compound 151. It (4.1 mg) was prepared from 148 following the same procedure described for the synthesis of 149.

### Experimental Procedures for the Synthesis of Neisseria Meningitidis Carbohydrates and Immune Response Studies

Compound 161. To a stirred solution of 160 (5.50 g, 13.77 mmol) and pyridine (6.6 mL, 82.62 mmol) in DCM (80 mL) was added dropwise chloroacetyl chloride (3.72 mL, 46.82 mmol) at 0° C. under Ar. After 3 h, the reaction mixture was diluted with DCM, washed with 10% aq. HCl, water, and brine, dried over Na₂SO₄, and then concentrated under vacuum. The residue was purified by silica gel column chromatography (EtOAc/hexane=1:3) to give the α,β-mixture of 161 (7.66 g, 89%) as a white solids.

Compound 162. A mixture of 161 (6.29 g, 10.0 mmol), dibutyl phosphate (5.0 mL, 25.0 mmol) and activated MS 4 Å (12.0 g) in anhydrous DCM (60 mL) was stirred under Ar atmosphere at rt for 1 h. After cooling to 0° C., NIS (3.38 g, 15.0 mmol) and TfOH (180 μL) were added, and the reaction was kept being stirred for 12 h. The reaction was quenched with aq. Na₂S₂O₃ and then filtered through a Celite pad. The filtrate was diluted with DCM and washed with saturated aq. NaHCO₃, water, and brine, dried over Na₂SO₄, and concentrated under vacuum. The residue was purified by silica gel column chromatography (EtOAc/Hexane=1:2) to give the α,β-mixture of compound 162 (7.05 g, 97%, α:β=1.6:1) as yellow solids.

General Procedure for Glycosylation Reactions Using Sialyl Phosphates as Glycosyl Donors.

A mixture of glycosyl donor (1.1 mmol), accepter (1 mmol), and activated MS 4 Å (1.0 g/mmol) in a mixture of anhydrous CH₃CN and DCM (V/V=1:2) was stirred under an Ar atmosphere at rt for 3 h. The mixture was cooled to −70° C., and TMSOTf (180 μL) was added. Then, the reaction solution was slowly warmed to −40° C. and stirred for 1 h. The reaction mixture was diluted with DCM and filtered through a Celite pad. The filtrate was washed with saturated aq. NaHCO₃, water, and brine, dried over Na₂SO₄, and concentrated under vacuum. The residue was purified by silica gel column chromatography to give the target compound.

Compound 163. It (2.26 g, 85%) was obtained from 162 (3.21 g, 4.39 mmol) and 2-azidoethanol (0.35 g, 4.0 mmol) using the above glycosylation method.

Compound 164. After 163 (2.43 g, 4.01 mmol) was dissolved in anhydrous MeOH (30 mL), triethylamine (0.3 mL) was added dropwise at rt. The reaction was stirred for 10 min, and was then quenched with 10% aq. HCl. The organic layer was isolated and concentrated, and the residue was subjected to silica gel column chromatography (MeOH/EtOAc/Hexane=1:5:5) to afforded 164 (1.39 g, 92%) as white solids.

Compound 165. A crude intermediate of trichloroacetyl-protected disialoside was obtained from donor 162 (1.02 g, 1.40 mmol) and sialoside triol 164 (0.48 g, 1.28 mmol) by means of the general glycosylation procedure. The intermediate was dissolved in anhydrous DCM (18 mL) and then cooled to 0° C. under N₂. Acetic anhydride (1.2 mL, 12.80 mmol) and TfOH (25 μL) were added and the mixture was stirred for 20 min. The reaction was quenched with saturated aq. NaHCO₃, diluted with EtOAc, and washed with water and brine, dried over Na₂SO₄, and then concentrated. The residue was added into a mixture of anhydrous MeOH (10.0 mL) and triethylamine (0.1 ml). The reaction was stirred at rt for 20 min, quenched with 10% aq. HCl, and concentrated under vacuum. The residue was finally purified by silica gel column chromatography to afforded 165 (0.79 g, 82%, three steps) as white solids.

Compound 166. To a stirred solution of 165 (80 mg, 0.11 mmol) in MeOH (5 ml) and H₂O (5 ml) was added LiOH.H₂O (62 mg, 1.47 mmol). After being refluxed for 24 h, the reaction mixture was concentrated under vacuum. The residue was dissolved in H₂O (6 ml), and then NaHCO₃ (247 mg, 2.94 mmol) and acetic anhydride (140 μL, 1.47 mmol) were added. After being stirred at rt for 3 h, the reaction mixture was concentrated under vacuum. The residue was dissolved in MeOH (5 mL), and then NaOMe (60 mg) was added. After being stirred at rt for 24 h, the reaction mixture was neutralized with 10% aq. HCl and concentrated under vacuum. The residue was dissolved H₂O (6 mL), and 10% Pd/C (20 mg) was added. The reaction mixture was shaken under a H₂ atmosphere at 50 psi for 12 h. The solid catalyst was removed by filtration through a Celite pad and the pad was washed with water. The filtrates were combined and concentrated under vacuum. The residue was purified by a sephadex G-10 column, using H₂O as the eluent, to give 166 (41 mg, 60%, 4 steps). ¹H NMR (600 MHz, D₂O): δ 3.85-3.60 (m, 7H), 3.59-3.34 (m, 9H), 3.00 (t, J=11.7 Hz, 2H), 2.54 (t, J=14.9 Hz, 3H), 1.87 (s, 6H), 1.54 (dd, J=25.0, 12.5 Hz, 3H). ¹³C NMR (150 MHz, D₂O): δ 174.9, 173.6, 173.4, 100.3, 100.1, 72.4, 72.4, 71.6, 70.2, 68.2, 68.0, 65.1, 62.5, 60.4, 51.8, 51.7, 40.1, 39.7, 39.4, 22.01, 21.96. HRMS (ESI-TOF, [M−H]⁻): calcd. for C₂₄H₄₀N₃O₁₇, 642.2358; found, 642.2361.

Compound 167. It (0.71 g, 88%, 3 steps) was prepared from 162 (0.57 g, 0.78 mmol) and 165 (0.54 g, 0.72 mmol) by the procedure described in the synthesis of 165.

Compound 168. It (55 mg, 66%, four steps) was prepared from 167 (100 mg, 89 μmol) by the procedure described in the synthesis of 166. ¹H NMR (600 MHz, D₂O): δ 3.87-3.62 (m, 10H), 3.61-3.37 (m, 13H), 2.99 (m, 2H), 2.60 (m, 3H), 1.88 (s, 9H), 1.55 (m, 3H). ¹³C NMR (150 MHz, D₂O): δ 174.9 (3C), 173.6, 173.4 (2C), 100.4, 100.2, 100.1, 72.4, 72.3, 72.2, 71.7, 70.2, 68.4, 68.3, 68.0, 65.1, 64.9, 62.6, 51.8, 51.8, 51.7, 40.0 (2C), 39.8, 39.5, 22.1 (2C), 22.0. HRMS (ESI-TOF, [M−H]⁻): calcd. for C₃₅H₅₇N₄O₂₅, 933.3312; found, 933.3293.

Compound 169. A crude intermediate of trichloroacetyl-protected trisialoside was prepared from 162 (107 mg, 0.15 mmol) and 167 (150 mg, 0.13 mmol) by means of the general glycosylation procedure. The resultant intermediate was dissolved in anhydrous DCM (5 mL), and then acetic anhydride (120 μL, 1.28 mmol) and TfOH (2.5 μL) were added at 0° C. After being stirred for 20 min, the reaction was quenched with saturated aq. NaHCO₃, and the mixture was diluted with EtOAc, washed with brine, and dried over Na₂SO₄. Concentration of the solution under vacuum and purification of the residue by silica gel column chromatography (MeOH/EtOAc/Hexane=1:10:10) afforded 169 (171 mg, 76%, two steps) as a white foamy solid.

170. It (22 mg, 62%, four steps) was prepared from 169 (50 mg, 29 μmol) by the procedure described in the synthesis of 15. ¹H NMR (600 MHz, D₂O): δ 3.82-3.62 (m, 13H), 3.58-3.42 (m, 15H), 3.38 (d, J=11.3 Hz, 2H), 2.95 (m, 2H), 2.60-2.50 (m, 4H), 1.88 (dd, J=14.5, 6.2 Hz, 12H), 1.54 (dd, J=23.4, 11.6 Hz, 4H). ¹³C NMR (151 MHz, D₂O): δ 174.9, 174.84, 174.81, 173.63, 173.59, 173.58, 173.4, 100.35, 100.14, 100.08, 72.4, 72.3, 72.2, 71.6, 70.2, 70.19, 70.1, 68.5, 68.3, 68.27, 68.25, 68.1, 68.03, 67.99, 65.1, 64.8, 62.6, 60.7, 51.82, 51.79, 51.74, 51.73, 40.0, 39.7, 39.4, 23.2, 22.1, 21.98. HRMS (ESI-TOF, [M−H]⁻): calcd. for C₄₆H₇₄N₅O₃₃, 1224.4266; found, 1224.4288.

171. The α,β-mixture of 171 (780 mg, 91%, two steps) was prepared from 162 (623 mg, 0.85 mmol) and 160 (354 mg, 0.85 mmol) by the procedure described for 169.

Compound 172. The α,β-mixture of 172 (543 mg, 87%, α/β=1.4/1) was prepared from 171 (565 mg, 0.85 mmol) by the procedure described for 162.

Compound 173. It (206 mg, 70%) was prepared from 172 (200 mg, 0.18 mmol) and 167 (157 mg, 0.14 mmol) by the procedure described for 169.

Compound 174. It (25 mg, 69%, four steps) was prepared from 173 (50 mg, 24 μmol) by the procedure described in the synthesis of 166. ¹H NMR (600 MHz, D₂O): δ 3.85-3.59 (m, 16H), 3.58-3.32 (m, 21H), 2.98 (s, 2H), 2.59-2.47 (m, 5H), 1.97-1.74 (m, 15H), 1.53 (dd, J=22.7, 10.9 Hz, 5H). ¹³C NMR (150 MHz, D₂O): δ 174.8 (4C), 174.8, 173.62, 173.58, 173.6, 173.4, 100.3, 100.1 (3C), 100.0, 72.4, 72.3, 72.2, 71.6, 70.2, 70.2, 70.1, 68.4, 68.3, 68.2, 68.1, 68.99, 67.96, 65.1, 65.0, 64.8, 62.5, 60.6, 51.78, 51.7, 40.0, 39.7 (3C), 39.4, 22.1 (4C), 22.0. HRMS (ESI-TOF, [M−H]⁻): calcd. for C₅₇H₉₁N₆O₄₁, 1515.5220; found, 1515.5216.

Procedure for the Activation of Oligosialic Acids:

A mixture of free oligosialic acids 166, 168, 170 or 174 (6.0 mg) and disuccinimidal glutarate (15 eq.) in DMF:PBS (0.1 M PBS buffer) (4:1, 0.5 mL) was stirred at rt for 4 h, and the solvents was then removed under vacuum. The activated oligosaccharides 175-178 were separated from the reagents by precipitation with 9 volumes of EtOAc, followed by washing of the precipitates 10 times with EtOAc and drying under vacuum, and were directly used for the next step without further purification.

Procedure for the Conjugation of 152-159 with HSA and KLH:

The activated oligosaccharides 175-178 were mixed with HSA or KLH at a molar ratio of 30:1 in 0.1 M PBS buffer (0.4 mL). The solution was stirred at rt for 3 days and then was applied to Biogel A 0.5 column using 0.1 M PBS buffer (I=0.1, pH=7.8) as the eluent to remove the free sugars. Fractions containing the glycoconjugate, characterized by the bicinchoninic acid (BCA) assay for proteins and charring with 15% (v/v) H₂SO₄ in EtOH for oligosialic acids, were combined and dialyzed against distilled water for 2 days, and then lyophilized to afford white solids of the desirable glycoconjugates.

Analysis of the Carbohydrate Loading of Glycoconjugates:

The mixture of an accurately weighted glycolconjugate sample (0.3-0.6 mg) in distilled water (1 mL) and the resorcinol reagent (2.0 mL) was heated in a boiling water bath for 30 min. After being cooled to room temperature, an extraction solution (3 mL of 1-butanol acetate and 1-butanol, v/v=85/15) was added. The mixture was shaken vigorously and subjected to stand for 10 min. The organic layer was transferred to a 1.0-cm cuvette, and the absorbance was determined at A580 nm by an UV-Vis spectrometer, using a blank extraction solution as the control. The sialic acid content of the glycoconjugate is determined by comparing the analyzed sample with a calibration curve created with the solution of standard sialic acid (NeuNAc) samples analyzed under the same condition. The sialic acid loading of each glycoconjugate was calculated according to the following equation, and the results are shown below.

$$Polysialic acid loading ( \% ) = \frac{sialic acid content ( mg ) in the sample}{weight of the glycocojugate sample ( mg )} \times 100 \%$$

Carbohydrate loadings of glycoconjugates 152-159

| | KLH conjugates | | | | HSA conjugates | | | |
|---|---|---|---|---|---|---|---|---|
| | Sample | | | | | | | |
| | 152 | 153 | 154 | 155 | 156 | 157 | 158 | 159 |
| Loading (%) | 7.5 | 11.5 | 7.9 | 6.8 | 8.9 | 11.5 | 10.9 | 7.8 |

Compound 184. A solution of disialic acid 166 (6.4 mg, 10 μmol), activated MPLA ester 6 (38 mg, 15.6 μmol), and a drop of N-methylmorpholine in anhydrous DMF (2 mL) was stirred at rt for 3 days. The reaction mixture was concentrated under vacuum, and the residue was purified on a preparative TLC plate (DCM/MeOH/H₂O/DMF=6:6:1:1) to give 184 (17.5 mg, 59.5%) as a white solid.

Compound 185. It (17.2 mg, 53.2%) was prepared from trisialic acid 168 (9.3 mg, 10 μmol) and activated ester 6 (38 mg, 15.6 μmol) by the same synthetic method described for 184.

Compound 186. It (14.8 mg, 42.0%) was prepared from tetrasialic acid 170 (12.3 mg, 10 μmol) and activated ester 6 (38 mg, 15.6 μmol) by the same synthetic method described for 184.

Compound 187. It (17.1 mg, 44.8%) was prepared from pentasialic acid 174 (15.2 mg, 10 μmol) and activated ester 6 (38 mg, 15.6 μmol) by the same synthetic method described for 184.

Compound 188. It (8.2 mg, 49.5%) was prepared from tetrasialic acid 170 (6.2 mg, 5 μmol) and activated ester 142 (17 mg, 7.8 μmol) by the same synthetic method described for 184.

Compound 179. A mixture of 184 (10.0 mg, 3.4 μmol) and 10% Pd/C (20.0 mg) in MeOH, DCM, and H₂O (5 ml, 6:6:1) was shaken under a hydrogen atmosphere (50 psi) for 24 h. The catalyst was removed by filtration through a Celite pad, and the pad was subsequently washed with mixtures of MeOH, DCM, and H₂O. The combined filtrates were concentrated to give 179 (8.0 mg, 98%) as an white solid. ₁H NMR (600 MHz, CDCl₃/CD₃OD/D₂O=3:3:1): δ 5.24 (t, J=9.5 Hz, 1H), 5.15-5.09 (m, 2H), 4.97 (t, J=8.4 Hz, 1H), 4.65 (d, J=8.2 Hz, 1H), 4.49 (d, J=8.2 Hz, 1H), 4.24 (m, 1H), 4.12 (d, J=10.5 Hz, 1H), 4.05-3.90 (m, 3H), 3.86-3.47 (m, 24H), 3.41-3.30 (m, 4H), 2.70 (m, 2H), 2.55-2.24 (m, 16H), 2.01 (m, 6H), 1.76-1.71 (m, 2H), 1.71-1.02 (m, 110H), 0.86 (t, J=6.7 Hz, 18H). ₃₁P NMR (161 MHz, CDCl₃/CD₃OD/D₂O=3:3:1): δ −0.55. MALDI-TOF MS: calcd. for C₁₁₈H₂₁₁N₆O₄₁P [M−2H]₂₋, 1199.72; found, 1199.82.

Compound 180. It (8.1 mg, 97%) was prepared from 185 (10 mg, 3.1 μmol) by the same synthetic method described for 179. ¹H NMR (600 MHz, CDCl₃/CD₃OD/D₂O=3:3:1): δ 5.25 (t, J=9.6 Hz, 1H), 5.14-5.09 (m, 2H), 4.98 (t, J=10.0 Hz, 1H), 4.64 (d, J=9.5 Hz, 1H), 4.50 (d, J=8.2 Hz, 1H), 4.29-4.22 (m, 1H), 4.10 (d, J=11.0 Hz, 1H), 4.04 (m, 3H), 3.88-3.48 (m, 30H), 3.43-3.32 (m, 4H), 2.76-2.67 (m, 3H), 2.54-2.25 (m, 15H), 2.21-2.16 (m, 1H), 2.07-1.95 (m, 9H), 1.90-1.06 (m, 113H), 0.98-0.74 (m, 18H). ³¹P NMR (161 MHz, CDCl₃/CD₃OD/D₂O=3:3:1): δ −0.56. MALDI-TOF MS: calcd. for C₁₂₉H₂₂₈N₇O₄₉P [M−2H]²⁻, 1345.27; found, 1345.27.

Compound 181. It (8.2 mg, 97%) was prepared from 186 (10 mg, 2.8 μmol) by the same synthetic method described for 179. ¹H NMR (600 MHz, CDCl₃/CD₃OD/D₂O=3:3:1): δ 5.25 (t, J=9.5 Hz, 1H), 5.13 (m, 2H), 4.98 (m, 1H), 4.66 (d, J=8.1 Hz, 1H), 4.50 (d, J=8.2 Hz, 1H), 4.23 (m, 1H), 4.12 (m, 1H), 4.04-3.96 (m, 4H), 3.88-3.47 (m, 37H), 3.41-3.33 (m, 4H), 2.70 (m, 4H), 2.54-2.25 (m, 16H), 2.01 (s, 12H), 1.81-1.09 (m, 114H), 0.96-0.76 (m, 18H). ³¹P NMR (161 MHz, CDCl₃/CD₃OD/D₂O=3:3:1): δ −0.56. MALDI-TOF MS: calcd. for C₁₄₀H₂₄₅N₈O₅₇P [M−2H]²⁻, 1490.81; found, 1490.94.

Compound 182. It (8.5 mg, 99%) was prepared from 187 (10 mg, 2.6 μmol) by the same synthetic method described for 179. ₁H NMR (600 MHz, CDCl₃/CD₃OD/D₂O=3:3:1): δ 5.25 (t, J=9.5 Hz, 1H), 5.11 (m, 2H), 4.98 (m, 1H), 4.63 (s, 1H), 4.25 (d, J=8.1 Hz, 1H), 4.03 (m, 4H), 3.91-3.49 (m, 44H), 3.37 (dd, J=33.4, 6.3 Hz, 4H), 2.72-2.66 (m, 5H), 2.39 (m, 16H), 2.01 (s, 15H), 1.88-1.02 (m, 115H), 0.94-0.78 (m, 18H). ³¹P NMR (161 MHz, CDCl₃/CD₃OD/D₂O=3:3:1): δ −0.55. MALDI-TOF MS: calcd. for C₁₅₁H₂₅₉N₉O₆₅P [M−5H+2Li]³⁻, 1094.57; found, 1094.59.

Compound 183. It (3.8 mg, 85%) was prepared from 188 (5 mg, 1.5 μmol) by the same synthetic method described for 179. ¹H NMR (600 MHz, CDCl₃/CD₃OD/D₂O=3:3:1): δ 5.30 (t, J=9.3 Hz, 1H), 5.23-5.20 (m, 1H), 5.12-5.09 (m, 2H), 4.47-4.45 (m, 1H), 4.22-4.21 (m, 1H), 4.05 (s, 3H), 3.91-3.46 (m, 41H), 2.71 (m, 4H), 2.50-2.24 (m, 15H), 2.17 (m, 1H), 1.97 (m, 12H), 1.91-0.98 (m, 114H), 0.92-0.76 (m, 18H). ³¹P NMR (161 MHz, CDCl₃/CD₃OD/D₂O 3:3:1): δ −0.58. MALDI-TOF MS: calcd. for C₁₄₀H₂₄₅N₈O₅₅P [M−2H]²⁻, 1474.82; found, 1474.85.

Protocols for vaccine formulation preparation. Liposomes of glycoconjugates 179-183 were prepared by a previously reported protocol. Briefly, after the mixture of a MPLA conjugate (0.42 μmol, that is, 1.01 mg of 179, 1.13 mg of 180, 1.25 mg of 181, 1.37 mg of 182, or 1.24 mg of 183, respectively), DSPC (2.15 mg, 2.7 μmol), and cholesterol (0.81 mg, 2.1 μmol) (in a molar ratio of 10:65:50) was dissolved in a mixture of CH2Cl2, MeOH, and H₂O (3:3:1, v/v, 2 mL), the solvents were removed under reduced pressure through rotary evaporation, which generated a thin lipid film on the vial wall. This film was hydrated by adding 3.0 mL of HEPES buffer (20 mM, pH 7.5) containing 150 mM of NaCl in a 60° C. water bath and then shaking the mixture on a vortex mixer. The resultant suspension was sonicated for 20 min to form liposomes used for immunizations. The size of the resulting liposomes was determined by dynamic light scattering (DLS) measurement, and their average diameter was about 1500 nm with a polydispersity index (PDI) of around 0.60.

The preparation of the CFA, Alum, and Titermax Gold Adjuvant emulsions of disialic acid-MPLA conjugate 179 followed the manufacturers' protocols. Generally, the liposomal preparation (0.75 mL, containing 0.51 mg of 1) obtained above was thoroughly mixed with an adjuvant (0.75 mL) to get each emulsion.

Immunization of mouse: Each mouse in a group of five or six was inoculated on day 1 via subcutaneous (s.c.) injection of a liposomal preparation of conjugates 179-183 (0.1 mL), respectively, for the initial immunization. In the CFA, Alum and Titermax groups, each mouse was inoculated via intramuscular (i.m.) injection of a specific emulsion of conjugate 189 (0.1 mL). Following the initial immunization, mice were boosted 3 times on day 14, day 21, and day 28 by s.c. injection of the same conjugate preparation (0.1 mL). Antisera were prepared from the blood samples of each mouse collected through the mouse leg veins prior to the initial immunization on day 0 and after immunization on day 28 and day 38 and stored at −80° C. before immunological analysis.

ELISA protocols: ELISA was performed by the same protocols used previously. ELISA plates were pre-coated with a solution of a specific oligosialic acid-HSA conjugate (100 μL, 2 μg sialic acid/mL) dissolved in the coating buffer (0.1 M bicarbonate, pH 9.6) at 37° C. for 1 h. After washing three times with phosphate-buffered saline (PBS) containing 0.05% Tween-20 (PBST), the plates were treated with a blocking buffer [10% bovine serum albumin (BSA) in PBST] at rt for 1 h. Thereafter, a pooled or an individual mouse antiserum solution (100 μL) with serial half-log dilutions from 1:300 to 1:656100 in PBS was added to each well of the plates, which was followed by incubation at 37° C. for 2 h. The plates were washed with PBST and then incubated at rt for 1 h with a 1:1000 diluted solution (100 μL/well) of AP-coupled goat anti-mouse kappa, IgM, IgG1, IgG2b, IgG2c, and IgG3 antibody, respectively. The plates were washed with PBST and then treated with a p-nitrophenylphosphate (PNPP) buffer solution (100 μL, 1.67 mg/mL) at rt for 30 min. The plates were finally examined using a microplate reader at 405 nm wavelength. The optical density (OD) values after deduction of background readings were plotted against the antiserum dilution numbers, and the equation of the best-fit line was obtained for each set of data and used to calculate the antibody titer of each sample. The antibody titer was defined as the dilution number giving an OD value of 0.2.

Assays of antiserum binding to N. meningitidis cell: Modified protocols for ELISA using a Bio-Dot microfiltration apparatus were employed to assess binding of antibodies in antisera to group C N. meningitidis cell. Briefly, the PVDF membrane was pre-treated in blocking buffer (1% BSA in PBST) and then set on the microfiltration apparatus to keep cells during the assays. A suspension of pre-killed N. meningitidis (ATCC® 31275™) cells (50 μL, OD 0.2 at 600 nm in PBS) was added in each well of the plate. After PBS buffer was removed through filtration, the bacterial cells remaining in the wells were incubated with a blocking buffer (1% BSA in PBST, 200 μL/well) at rt for 1 h to block any nonspecific binding sites left on the surface of bacterial cells, and the blocking buffer was removed through filtration under vacuum. Thereafter, the plate was washed with PBST (350 μL) three times, followed by addition of 100 μL of normal mouse sera or pooled antisera (1:100 dilution in PBS) obtained with conjugates 179-182 to each well. The plate was incubated at 37° C. for 2 h and washed six times with PBST (350 μL). Then, to each well was added a 1:1000 diluted solution of AP-conjugated goat anti-mouse kappa antibody (100 μL/well), and the plate was incubated at rt for 1 h. Finally, the plate was washed with PBST six times, and then developed with a PNPP solution (1.67 mg/mL in buffer, 200 μL) at rt for 30 min. An aliquot of the solution (100 μL) was transferred from each well to a clear round-bottom 96-well plate for colorimetric reading at 405 nm wavelength with a microplate reader. The binding between antibodies and cells was reflected by the observed OD value for each well.

1. A compound of formula (I):
M-L-A (I)
wherein:
M is selected from the group consisting of a lipid A derivative and a protein;
L is a linker; and
A is a carbohydrate antigen comprising fucose, the carbohydrate being synthetic.

2. (canceled)

3. The compound of claim 1, wherein the carbohydrate is a globo H derivative.

4. The compound of claim 1, wherein the carbohydrate is 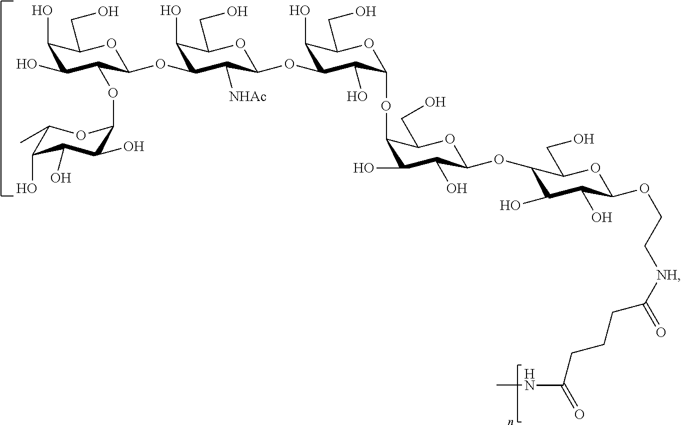
and wherein n represents an integer from 1 to 10 inclusive.

5. (canceled)

6. The compound of claim 1, wherein M comprises monophosphorylated lipid A.

7. The compound of claim 6, wherein the monophosphorylated lipid A is synthetic.

8. The compound of claim 1, wherein M comprises a protein selected from the group consisting of keyhole limpet cyanin, human serum albumin, tetanus toxoid, diphtheria toxin cross-reacting material 197, and diphtheria toxin.

9. (canceled)

10. The compound of claim 1, wherein L is selected from the group comprising:
—(C₁-C₁₀ alkyl)-X—Y—(C₁-C₁₀ alkyl)-F-G-, wherein F, G, X, and Y are each independently selected from the group consisting of C₁-C₁₀ alkyl, amide, carbonyl, alkene, cyano, phospho, and thio; and
at least one of 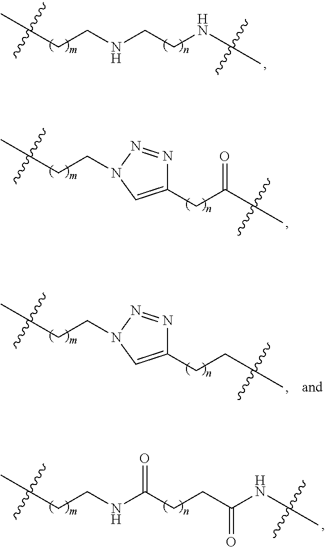
wherein m and n are each independently integers from 1-10 inclusive.

11. The compound of claim 10, wherein X and G are each amide and Y and F are each carbonyl.

12. The compound of claim 10, wherein the carbohydrate is selected from the group consisting of:

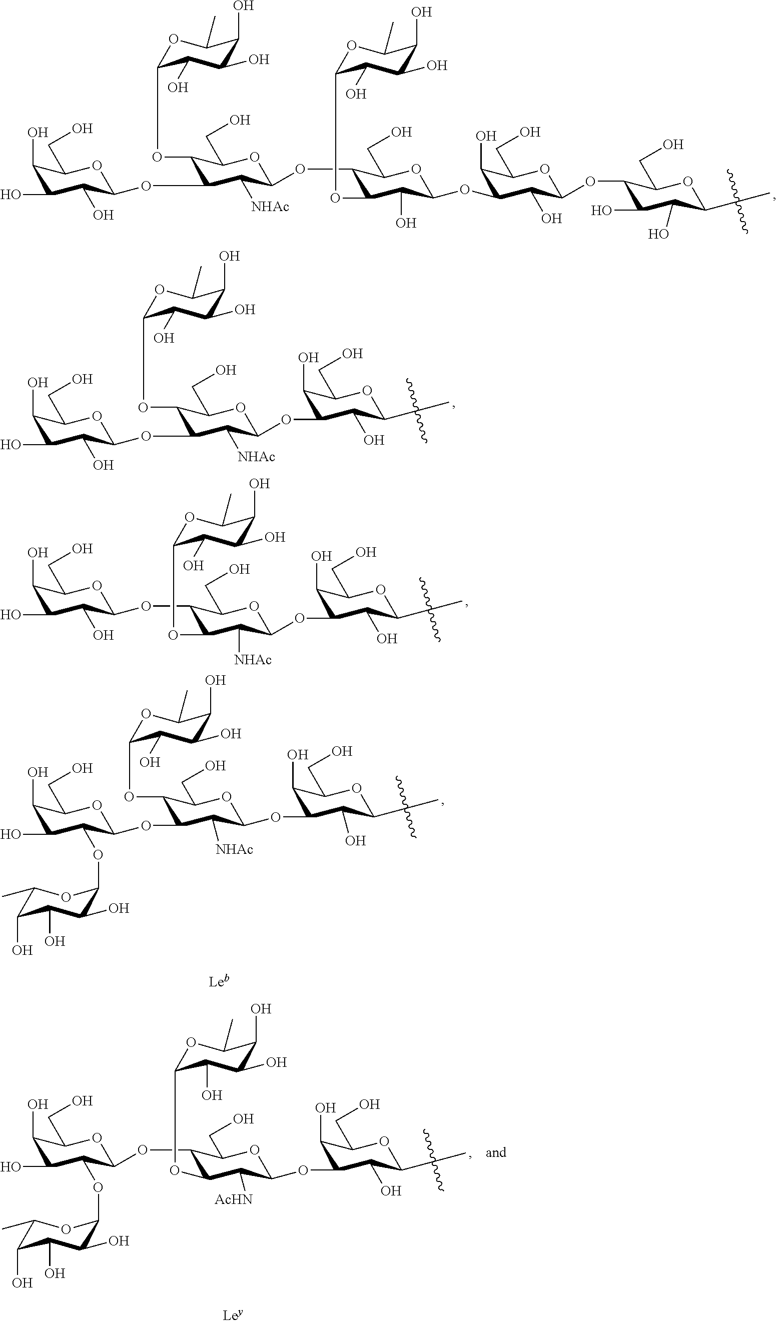

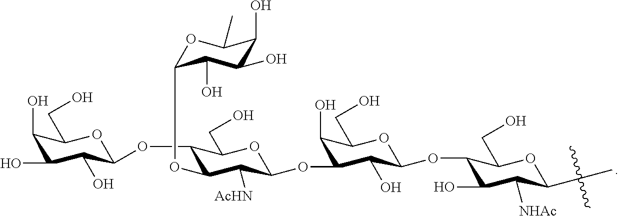

13.-16. (canceled)

17. A method of using a compound of claim 1 in a vaccine to treat a patient in need thereof.

18. The method of claim 17 wherein the vaccine is self-adjuvanting and synthetic.

19. The method of claim 17, wherein the vaccine is used to treat or prevent cancer.

20. (canceled)

21. A compound of formula III:
M-L-D formula III
wherein D is selected from the group consisting of an oligo-beta-glucan, an oligosialic acid chain, and an oligoribosylribitol phosphate;
L comprises a linker; and
M is selected from one of a monophosphorylated lipid A derivative and a carrier protein.

22. The compound of claim 21 comprising a plurality of beta-glucan units in a chain.

23.-35. (canceled)

36. A method of using a compound of claim 22 in a vaccine to treat a patient in need thereof.

37. (canceled)

38. (canceled)

39. The method of claim 36, wherein the vaccine is used to treat or prevent a fungal disease.

40. (canceled)

41. The compound of
claim 21, wherein D comprises an oligosialic acid chain.

42.-59. (canceled)

60. A method comprising using a compound of claim 41 in a vaccine, wherein the vaccine is used to treat or prevent a bacterial infection.

61. compound of claim 21,
wherein D comprises an oligoribosylribitol phosphate.

62.-76. (canceled)

77. A method comprising using a compound of claim 61 in a vaccine, wherein the vaccine is used to treat or prevent a bacterial infection or a bacterial disease.

78. (canceled)


SEQUENCE LISTING (1 sequence)

SEQ ID NO: 1 - protein, 609 aa, Homo Sapiens [sequence omitted]